(19) United States
(12) Patent Application Publication
(10) Pub. No.: US 2026/0092274 A1
(43) Pub. Date: Apr. 2, 2026

(54) METHODS OF SCREENING AND EXPRESSING CIS-DISPLAY LIBRARIES OF DISULFIDE-RICH POLYPEPTIDES

(71) Applicant: Applied Biomedical Science Institute, San Diego, CA (US)

(72) Inventors: Duncan MCGREGOR, San Diego, CA (US); Gabrielle WARNER JENKINS, San Diego, CA (US); Vaughn SMIDER, San Diego, CA (US)

(73) Assignee: Applied Biomedical Science Institute, San Diego, CA (US)

(21) Appl. No.: 19/342,407

(22) Filed: Sep. 26, 2025

Related U.S. Application Data
(60) Provisional application No. 63/700,581, filed on Sep. 27, 2024.

(51) Int. Cl. C12N 15/10

(52) U.S. Cl. CPC C12N 15/1093

(57) ABSTRACT

The present disclosure relates to methods of producing and screening libraries of disulfide rich proteins, for instance to identify binding peptides specific for a target molecule. In some embodiments, the binding peptides comprise disulfide rich proteins. Also provided herein are methods of producing and screening libraries comprising disulfide-rich proteins and molecular chaperones. The present disclosure also relates to methods of producing or expressing soluble disulfide rich proteins, for instance using a suitable host cell. Also provided herein are compositions comprising soluble disulfide rich proteins.

### CROSS-REFERENCE TO RELATED APPLICATIONS

This application claims priority from U.S. provisional application No. 63/700,581, filed Sep. 27, 2024, entitled “METHODS OF SCREENING AND EXPRESSING CIS-DISPLAY LIBRARIES OF DISULFIDE-RICH POLYPEPTIDES”, the contents of which are incorporated by reference in their entirety.

### STATEMENT OF GOVERNMENT INTEREST

This invention was made with government support under R01 AI173109 awarded by the National Institutes of Health. The government has certain rights in the invention.

### INCORPORATION BY REFENCE OF SEQUENCE LISTING

The present application is being filed along with a Sequence Listing in electronic format. The Sequence Listing is provided as a file entitled 16577-2000700.xml created Sep. 26, 2025, which is 107,019 bytes in size. The information in the electronic format of the Sequence Listing is incorporated by reference in its entirety.

### FIELD

The present disclosure relates to methods of producing and screening DNA libraries of disulfide-rich polypeptides, for instance to identify polypeptides with increased binding affinity for a target molecule. In some embodiments, the binding polypeptides comprise disulfide-rich proteins. Also provided herein are DNA libraries comprising disulfide-rich polypeptides and chaperone proteins. The present disclosure also relates to methods of producing or expressing soluble disulfide-rich polypeptides, for instance using a suitable host cell. Also provided herein are compositions comprising soluble disulfide-rich binding polypeptides.

### BACKGROUND

CIS display is a library screening strategy for identifying binding molecules or binding ligands. It exploits the DNA replication initiator protein (RepA) to bind to template DNA to facilitate in vitro transcription and translation and to create a library of protein-DNA complexes that can be screened for binding activity. While useful for short linear proteins, the methods are not entirely satisfactory for screening many peptides and proteins. Improved methods are needed.

### SUMMARY

Provided herein is a method of producing a disulfide rich protein (DRPs) CIS display library. In some embodiments, methods of producing a DRP CIS display library comprises (a) providing a plurality of DNA constructs each DNA construct comprising: (i) a DNA sequence encoding a DRP, (ii) one or more DNA sequences encoding a molecular chaperone, (iii) a DNA sequence encoding a DNA replication initiator protein, and (iv) a DNA sequence encoding a target sequence for the DNA replication initiator protein, wherein the DNA replication initiator protein can non-covalently bind to the target sequence; (b) expressing the plurality of DNA constructs of (a) in a prokaryotic cell-free transcription/translation environment, thereby producing a plurality of DRP-chaperone-DNA replication initiator proteins from the plurality of DNA constructs; and (c) linking each of the DRP-chaperone-DNA replication initiator proteins expressed in (b) to its corresponding DNA construct by binding of the DNA replication initiator protein to the target sequence in the corresponding DNA construct, thereby producing the DRP CIS display library.

Provided herein is a method of producing a disulfide rich protein (DRPs) CIS display library, the method comprising expressing a plurality of DNA constructs in a prokaryotic cell-free transcription/translation environment in the presence of at least one molecular chaperone, wherein the at least one molecular chaperone is provided by (i) expression from a DNA sequence encoding the molecular chaperone included in the plurality of DNA constructs, or (ii) addition of one or more DNA sequences encoding the molecular chaperone or the molecular chaperone itself to the transcription/translation environment, wherein each DNA construct comprises: i. a DNA sequence encoding one of a plurality of disulfide rich protein (DRP); ii. a DNA sequence encoding a DNA replication initiator protein; and iii. a DNA sequence comprising a target sequence for the DNA replication, thereby producing a plurality of DNA:protein fusion proteins comprising the DRP and the DNA replication initiator protein and, optionally the molecular chaperone, from the plurality of DNA constructs, wherein each fusion protein binds, via the DNA replication initiator protein, to the target sequence in the DNA construct from which it was transcribed, thereby linking the fusion protein to the DNA construct to produce the DRP CIS display library comprising a plurality of DNA:protein fusion proteins.

In some of any of the provided embodiments, the molecular chaperone is provided by expression from a DNA sequence encoding the molecular chaperone included in each of the DNA constructs, and wherein each of the plurality of DNA:protein fusion proteins comprises a DRP, the molecular chaperone, and the DNA replication initiator protein.

Provided herein is a method of producing a disulfide rich protein (DRPs) CIS display library, the method comprising: (a) providing a plurality of DNA constructs each DNA construct comprising: i. a DNA sequence encoding one of a plurality of disulfide rich proteins (DRPs); ii. one or more DNA sequences encoding a molecular chaperone; iii. a DNA sequence encoding a DNA replication initiator protein; and iv. a DNA sequence comprising a target sequence for the DNA replication initiator protein, wherein the DNA replication initiator protein can non-covalently bind to the target sequence; and (b) expressing the plurality of DNA constructs of (a) in a prokaryotic cell-free transcription/translation environment, thereby producing a plurality of DNA:protein fusion proteins comprising the DRP, the molecular chaperone and the DNA replication initiator protein from the plurality of DNA constructs, and wherein each fusion protein binds, via the DNA replication initiator protein, to the target sequence in the DNA construct from which it was transcribed, thereby linking the fusion protein to the DNA construct to produce the DRP CIS display library comprising a plurality of DNA:protein fusion proteins.

In some of any of the provided embodiments, the method comprises the addition of one or more DNA sequences encoding the molecular chaperone or the molecular chaperone itself to the transcription/translation environment, wherein each of the plurality of DNA:protein fusion proteins comprises a DRP and the DNA replication initiator protein.

Provided herein is a method of producing a disulfide rich protein (DRPs) CIS display library, the method comprising: (a) providing a plurality of DNA constructs each DNA construct comprising: i. a DNA sequence encoding one of a plurality of disulfide rich proteins (DRPs); ii. a DNA sequence encoding a DNA replication initiator protein; and iii. a DNA sequence comprising a target sequence for the DNA replication initiator protein, wherein the DNA replication initiator protein can non-covalently bind to the target sequence; (b) expressing the plurality of DNA constructs of (a) in a prokaryotic cell-free transcription/translation environment, thereby producing a plurality of DNA:protein fusion proteins comprising the DRP and the DNA replication initiator protein from the plurality of DNA constructs; c) adding a molecular chaperone, or one or more DNA sequences encoding a molecular chaperone, to the prokaryotic cell-free transcription/translation environment, during the expressing the plurality of DNA constructs in (b); wherein each fusion protein binds, via the DNA replication initiator protein, to the target sequence in the DNA construct from which it was transcribed, thereby linking the fusion protein to the DNA construct to produce the DRP CIS display library comprising a plurality of DNA:protein fusion proteins.

In some embodiments, the DRP has four or more cysteine residues for forming two or more disulfide bonds. In some embodiments, each of the plurality of DNA constructs comprise a DNA sequence encoding a different DRP. In some embodiments, the encoded DRP comprises a natural and/or synthetic DRP. In some embodiments, the encoded DRP comprises an antibody, an scFv fragment, a cyclotide, a knottin, a toxin, and a CDR3 knob domain from an ultralong CDR3, such as a cow CDR3 knob domain. In some embodiments, the encoded DRPs comprise DRP variants comprising one or more differences in their amino acid residues.

In some embodiments, the plurality of DRPs comprise a peptide of 20-70 amino acids in length having 2-12 cysteine residues capable of forming 1-6 intramolecular disulfide bonds. In some embodiments, the peptide comprises 4-8 cysteine residues forming 2-4 intramolecular disulfide bonds.

In some embodiments, the peptide is a knob domain derived from an ultralong CDR3 of an antibody. In some embodiments, the ultralong CDR3 of an antibody is from a member of the Bovinae subfamily. In some embodiments, the ultralong CDR3 of an antibody is from a bovine, optionally Bos taurus. In some embodiments, the DRP is a cow CDR3 knob.

In some embodiments, the one or more encoded molecular chaperones comprise a bacterial or eukaryotic origin. In some embodiments, the one or more encoded molecular chaperones comprise a molecular weight of 10 kD to 80 kD. In some embodiments, the one or more encoded molecular chaperones comprise a molecular weight of 10 kD to 24 kD. In some of any of the provided embodiments, the one or more encoded molecular chaperones comprise trxA, DsbA, DsbB, DsbC, HSP70, DnaK, HSP90, GroES, GroEL, DNAK, Sumo and Trigger Factor, or protein disulfide isomerase (PDI). In some embodiments, the one or more encoded molecular chaperones comprise DsbA, DsbB, DsbC, trxA, HSP70, and/or DnaK. In some embodiments, the one or more molecular chaperone is the molecular chaperone DsbA. In some embodiments, the one or more molecular chaperone is the molecular chaperone trxA.

In some embodiments, each DRP of the CIS display library has formed two or more disulfide bonds.

In some of any of the provided embodiments, the plurality of DNA constructs encodes the DRP, the one or more molecular chaperone, the DNA replication initiator protein, and the target sequence, in-frame. In some embodiments, the DNA construct encodes the DRP, the one or more molecular chaperones, the DNA replication initiator protein, and the target sequence in-frame. In some embodiments, the one or more encoded molecular chaperones are encoded upstream and/or downstream of the DRP in the DNA construct. In some embodiments, the one or more encoded molecular chaperones are encoded upstream of the DRP in the DNA construct.

In some embodiments, each of the plurality of DNA constructs comprises in order: the DNA sequence encoding the molecular chaperone, the DNA sequence encoding the DRP, the DNA sequence encoding the DNA replication initiator protein, and the DNA sequence encoding the target sequence for the DNA replication initiator protein.

In some embodiments, each of the plurality of DNA constructs further comprises a first linker sequence between the molecular chaperone and the DRP and/or a second linker sequence between the DRP and the DNA replication initiator protein. In some embodiments, the first linker is SEQ ID NO: 15. In some embodiments, the second linker is SEQ ID NO: 15.

Also provided herein is a polynucleotide comprising a nucleic acid sequence encoding a disulfide rich protein (DRP), a nucleic acid sequence encoding one or more molecular chaperone, a nucleic acid sequence encoding a DNA replication initiator protein, and a nucleic acid sequence comprising a target sequence for the DNA replication initiator protein, wherein the DNA replication initiator protein can non-covalently bind to the target sequence.

Provided herein is a polynucleotide comprising: (a) a nucleic acid sequence encoding a disulfide rich protein (DRP); (b) a nucleic acid sequence encoding a DNA replication initiator protein; and (c) a nucleic acid sequence comprising a target sequence for the DNA replication initiator protein, wherein the DNA replication initiator protein can non-covalently bind to the target sequence.

In some embodiments, the nucleic acid sequence encoding the DRP has four or more cysteine residues for forming two or more disulfide bonds.

In some embodiments, the nucleic acid sequence encoding the DRP comprises natural and/or synthetic DRPs. In some embodiments, the nucleic acid sequence encoding the DRP comprises an antibody, an scFv fragment, a cyclotide, a knottin, a toxin, and a cow CDR3 knob domain.

In some embodiments, the nucleic acid sequence encoding the DRP comprises sequence variants that encode one or more differences in amino acid residues. In some embodiments, the polynucleotide encodes the DRP sequence, the one or more molecular chaperone sequence, the DNA replication initiator protein sequence, and the target sequence in-frame. In some embodiments, the one or more molecular chaperone nucleic acid sequences are upstream or downstream of the DRP nucleic acid sequence in the polynucleotide. In some embodiments, the one or more molecular chaperone nucleic acid sequences are upstream of the DRP nucleic acid sequence in the polynucleotide.

In some embodiments, the one or more molecular chaperone nucleic acid sequences comprise a bacterial or eukaryotic origin. In some embodiments, the one or more molecular chaperone comprises a molecular weight of 10 kD to 80 kD. In some embodiments, the one or more molecular chaperone comprises a molecular weight of 10 kD to 24 kD. In some of any of the provided embodiments, the one or more molecular chaperone comprises trxA, DsbA, DsbB, DsbC, HSP70, DnaK, HSP90, GroES, GroEL, DNAK, Sumo and Trigger Factor, or protein disulfide isomerase (PDI). In some embodiments, the one or more molecular chaperone comprises DsbA, DsbB, DsbC, trxA, HSP70, and/or DnaK. In some embodiments, the one or more molecular chaperone sequences are the molecular chaperone DsbA. In some embodiments, the one or more molecular chaperone sequences are the molecular chaperone trxA.

In some embodiments, the polynucleotide comprises in order: the nucleic acid sequence encoding the molecular chaperone, the nucleic acid sequence encoding the DRP, the nucleic acid sequence encoding the DNA replication initiator protein, and the nucleic acid sequence comprising the target sequence for the DNA replication initiator protein. In some embodiments, the polynucleotide further comprises a sequence encoding a first linker sequence between the molecular chaperone and the DRP and/or a sequence encoding a second linker sequence between the DRP and the DNA replication initiator protein. In some embodiments, the first linker is SEQ ID NO: 15. In some embodiments, the second linker is SEQ ID NO: 15.

Also provided herein is a DNA construct comprising the polynucleotides of the aforementioned embodiments. In some of any of the provided embodiments, the composition further comprises a nucleic acid sequence encoding one or more molecular chaperone.

Provided herein is a DNA construct comprising any of the provided polynucleotides. Also provided herein is an expression vector comprising the polynucleotides of the aforementioned embodiments.

Also provided herein is a DNA:protein fusion comprising a protein comprising (i) a disulfide rich protein (DRP), (ii) one or more molecular chaperone proteins, and (iii) a DNA replication initiator protein, and a DNA comprising (i) a DNA sequence encoding the DRP, (ii) one or more DNA sequences encoding the molecular chaperone, (iii) a DNA sequence encoding the DNA replication initiator protein, and (iv) a DNA sequence comprising a target sequence for the DNA replication initiator protein, wherein the DNA replication initiator protein non-covalently binds to the DNA sequence encoding the target sequence, thereby producing the DNA:protein fusion.

Provided herein is a DNA:protein fusion comprising: (a) a protein comprising: i. a disulfide rich protein (DRP); and ii. a DNA replication initiator protein; and (b) a DNA comprising: i. a DNA sequence encoding the DRP; ii. a DNA sequence encoding the DNA replication initiator protein; and iii. a DNA sequence comprising a target sequence for the DNA replication initiator protein; wherein the DNA replication initiator protein non-covalently binds to the DNA sequence encoding the target sequence, thereby producing the DNA:protein fusion.

In some embodiments, the DRP comprises a natural and/or synthetic DRP. In some embodiments, the DRP comprises an antibody, an scFv fragment, a cyclotide, a knottin, a toxin, and a cow CDR3 knob domain.

In some embodiments, the one or more molecular chaperones comprise a bacterial or eukaryotic origin. In some embodiments, the one or more molecular chaperones comprise a molecular weight of 10 kD to 80 kD. In some embodiments, the one or more molecular chaperones comprise a molecular weight of 10 kD to 24 kD. In some of any of the provided embodiments, the one or more molecular chaperones comprise trxA DsbA, DsbB, DsbC, HSP70, DnaK, HSP90, GroES, GroEL, DNAK, Sumo and Trigger Factor, or protein disulfide isomerase (PDI). In some embodiments, the one or more molecular chaperones comprise DsbA, DsbB, DsbC, trxA, HSP70, and/or DnaK. In some embodiments, the one or more molecular chaperone is the molecular chaperone DsbA. In some embodiments, the one or more molecular chaperone is the molecular chaperone trxA.

In some embodiments, the DRP protein has formed two or more disulfide bonds.

In some embodiments, the one or more molecular chaperones are encoded upstream and/or downstream of the DRP in the DNA. In some embodiments, the one or more molecular chaperones are encoded upstream of the DRP in the DNA.

In some embodiments, the DNA comprises in order: the DNA sequence encoding the molecular chaperone, the DNA sequence encoding the DRP, the DNA sequence encoding the DNA replication initiator protein, and the DNA sequence encoding the target sequence for the DNA replication initiator protein. In some embodiments, the DNA further comprises a first linker sequence between the molecular chaperone and the DRP and/or a second linker sequence between the DRP and the DNA replication initiator protein. In some embodiments, the first linker is SEQ ID NO: 15. In some embodiments, the second linker is SEQ ID NO: 15.

Also provided herein is a screening library produced according to the aforementioned methods, comprising a DNA:protein fusion comprising a DRP-chaperone-DNA replication initiator protein bound to the corresponding DNA construct encoding said DRP-chaperone-DNA replication initiator protein sequence. Also provided herein is a screening library produced according to the aforementioned methods, comprising a DNA:protein fusion comprising a DRP-DNA replication initiator protein bound to the corresponding DNA construct encoding said DRP-DNA replication initiator protein sequence. In some embodiments, the screening library comprises 10⁶ to 10¹⁴ different DNA:protein fusions. In some embodiments, the screening library comprises 10¹⁰ to 10¹⁴ different DNA:protein fusions.

Also provided herein is a method of screening a disulfide rich protein (DRP) CIS display library, the method comprising providing a screening library produced according to the aforementioned methods, wherein each protein of a plurality of DRP-chaperone-DNA replication initiator proteins is linked to its corresponding DNA sequence, contacting the screening library with a target of interest to produce a mixture of DRP-chaperone-DNA replication initiator protein DNA:protein fusions and the target of interest, and selecting from the mixture the DRP-chaperone-DNA replication initiator protein DNA:protein fusions that are bound to the target of interest.

Provided herein is a method of screening a disulfide rich protein (DRP) CIS display library, the method comprising providing a screening library produced according to the aforementioned methods, wherein each protein of a plurality of DRP-DNA replication initiator proteins is linked to its corresponding DNA sequence, contacting the screening library with a target of interest to produce a mixture of DRP-DNA replication initiator protein DNA:protein fusions and the target of interest, and selecting from the mixture the DRP-DNA replication initiator protein DNA:protein fusions that are bound to the target of interest.

In some embodiments, the method further comprises identifying the one or more DRP proteins bound to the target of interest by identifying a sequence of the corresponding DRP-chaperone-DNA replication initiator protein DNA. In some of any of the provided embodiments, the method further comprises identifying the one or more DNA:protein fusion proteins bound to the target of interest by identifying a sequence of the corresponding DNA:protein fusion replication initiator protein DNA.

In some embodiments, the one or more encoded molecular chaperones comprise a bacterial or eukaryotic origin. In some embodiments, the one or more encoded molecular chaperones comprise a molecular weight of 10 kD to 80 kD. In some embodiments, the one or more encoded molecular chaperones comprise a molecular weight of 10 kD to 24 kD. In some embodiments, the one or more encoded molecular chaperones comprise DsbA, DsbB, DsbC, trxA, HSP70, and/or DnaK. In some embodiments, the one or more molecular chaperone is the molecular chaperone DsbA. In some embodiments, the one or more molecular chaperone is the molecular chaperone trxA.

In some embodiments, each DRP of the CIS display library has formed two or more disulfide bonds.

In some embodiments, the method further comprises screening the DRP CIS display library by contacting the expressed plurality of DNA constructs with a lysate comprising one or more molecular chaperones.

In some embodiments, identifying the sequence of the corresponding DRP-chaperone-DNA replication initiator protein DNA comprises nucleic acid sequencing. In some of any of the provided embodiments, the method comprises identifying the sequence of the corresponding DNA:protein fusion replication initiator protein DNA which comprises nucleic acid sequencing.

### BRIEF DESCRIPTION OF FIGURES

FIGS. 1A-1B depict exemplary schematics comparing the DNA-based in vitro CIS display systems. The displayed disulfide-rich proteins (DRPs) encoded by the ‘Library’ component are correctly folded when encoded in a complex with one or more chaperone proteins (e.g., trxA, DsbA, DsbB, and DsbC), allowing the displayed DRPs to properly bind to their targets (FIG. 1A). The traditional CIS display system does not include chaperone proteins, which leads to misfolded DRPs with inadequate binding capabilities (FIG. 1B).

FIG. 2 depicts a schematic of an exemplar DNA vector, pREPTRX (SEQ ID NO: 1), which can be used to display disulfide-rich proteins or protein fragments, a chaperone gene (e.g., TrxA (SEQ ID NO: 11)), and DNA elements required for CIS activity (e.g., RepA (SEQ ID NO: 8), CIS (SEQ ID NO: 9), and ORI (SEQ ID NO: 10)). Key primer sites and restriction enzyme sites are indicated on the schematic.

FIG. 3 depicts an agarose gel of amplified DNA products from recovered bound DNA after incubating DRP-DNA complexes with selected targets. Cow CDR3 knobs were expressed using DNA-based in vitro CIS display systems, including the improved CIS display system comprising one or more molecular chaperone. The in vitro transcription/translation products were mixed with the selection targets. Only products bound to the targets, which include anti-DO1, WT RBD, and WT Spike were isolated. Lanes labeled ‘A’ represent DRPs-DNA complexes expressed without a chaperone protein (e.g., Minus TrxA). Lanes labeled ‘B’ represent DRPs-DNA complexes expressed with a chaperone protein (e.g., Plus TrxA).

FIG. 4 depicts an agarose gel of amplified DNA products from recovered bound DNA after incubating DRP-chaperone-RepA DNA:protein complexes with selected targets. scFv fragments specific for αvβ3 integrin (SEQ ID NO: 6) and R2G3 specific for SARS2 RBD protein were expressed using DNA-based in vitro CIS display systems encoding a chaperone protein (e.g., DRP-DNA-chaperone complex). The in vitro transcription/translation products were mixed with the selection targets and the products bound to αvβ3 integrin or SARS2 RBD protein were isolated. Products associated with scFv or R2G3 knob are highlighted on the gel. The lane labeled ‘αvβ3’ represents DRPs-chaperone-RepA complexes selected with αvβ3 integrin protein, and the lane labeled ‘RBD’ represents DRPs-chaperone-RepA complexes selected with SARS2 RBD protein.

FIGS. 5A-5B depict binding of different clones expressing a variant from a library of DRP-chaperone-RepA DNA:protein complexes against the RBD domain of the SARS CoV-2 spike protein via ELISA. Quantified binding of the clones to RBD (FIG. 5A) and to human IgG (FIG. 5B; negative control) is shown.

FIGS. 6A-6B depict sequence and binding information for the most frequently isolated clones of DRPs, isolated from the recovered DNA of bound DRP-chaperone-RepA. FIG. 6A shows the most frequently detected sequences after three rounds of selection using the R2G3 variant DRP library. FIG. 6B shows representative sequences and ELISA binding values (OD 450 nm) for the individual clones after three rounds of selection.

FIGS. 7A-7B depicts binding of different clones expressing a variant from a library of CDR3 knob DNA-chaperone complexes against the MERS virus spike trimer protein via ELISA. Quantified binding of the clones to MERS spike trimer protein (FIG. 7A) and non-specific binding in a blank plate (FIG. 7B) as measured via ELISA is shown. The clones were isolated after three rounds of selection.

### DETAILED DESCRIPTION

Provided herein in some aspects are CIS display libraries and methods of producing and preparing CIS display libraries for screening of disulfide rich proteins (DRPs). Also provided herein in some aspects are methods of producing and screening DNA libraries of DRPs, for instance to identify DRPs with increased binding affinity for a target molecule. In some aspects, the DRP CIS display libraries encode disulfide rich proteins (DRPs). In provided embodiments, the methods use molecular chaperones to facilitate correct folding of DRPs in a cell-free transcription/translation environment for producing the CIS display library. The chaperone may be supplied in a variety of forms, including as a coding sequence present within the same DNA construct that encodes the DRP, as a separate nucleic acid added to the transcription/translation mixture, or as the chaperone protein itself added exogenously. By providing the chaperone in cis or in trans, proper oxidative folding and disulfide bond formation of DRPs can be achieved, thereby improving the yield, structural integrity, and functional display of DRPs in the library.

In some aspects, the DRP CIS display libraries encode DRPs, including but not limited to antibodies, antibody fragments, knottins, cyclotides, toxins, and CDR3 knob domain peptides, such as those derived from ultralong CDR3 from species of the Bovinae subfamily including bovine CDR3 knob domain peptides, for example cow CDR3 knob domain peptides. The DRPs are expressed as fusions with a DNA replication initiator protein, such as RepA, which binds to a cognate non-coding origin of replication sequence within the same DNA construct, thereby creating a physical linkage between each DRP and its encoding DNA. In provided embodiments, the methods disclosed herein further comprise expressing or adding a molecular chaperone as described herein to ensure correct folding of the DRP component of the fusion protein. The resulting CIS display libraries may then be screened for DRPs with desirable functional properties, such as binding affinity, stability, or specificity for a target molecule.

In some embodiments, the molecular chaperone is included as a coding sequence as part of the DNA construct encoding the DRP and replication initiation protein, e.g., RepA. As a result, the produced DRP library members of the CIS display library include the encoded molecule chaperone. In some aspects, the CIS display libraries encode one or more molecular chaperone proteins at the N- and/or C-terminus of the DRP. In some embodiments, the CIS display libraries encode one or more chaperone proteins at the N-terminus of the DRP. In some embodiments, the CIS display libraries encode one or more chaperone proteins at the C-terminus of the DRP. Also provided herein in some embodiments are methods of producing soluble peptides, in some instances producing soluble DRPs. The DRPs produced can be derived from natural sequences (e.g. from bovine) or synthetic. In some embodiments, the DRPs produced according to the provided methods can include CDR3 knobs, scFv, and cyclotides. In some embodiments, the DRP is a CDR3 knob peptide derived from an ultralong CDR3 of an antibody, such as those from species of the Bovinae subfamily for example from bovine (e.g., cow) ultralong CDR3 antibodies.

The provided embodiments relate to an improved method of building and screening CIS Display libraries, originally developed by Odegrip et al. 2004, Proc Natl Acad Sci USA. 2004 Mar. 2; 101(9): 2806-10. The original method works by creating libraries of peptides by the genetic fusion of synthetic peptide libraries to the N-terminus of RepA and performing a coupled in vitro transcription-translation reaction (ITT) in an E. coli S30 lysate, to express the peptide-RepA fusion protein and link the expressed protein to the DNA fragment from which it was transcribed and translated. The bacterial lysate is a reducing environment, preventing the correct formation of disulfide bonds, resulting in instability and aggregation of disulfide-rich protein and limiting its use to short linear peptides. Thus, existing CIS Display methods are not suitable for use with disulfide-rich proteins.

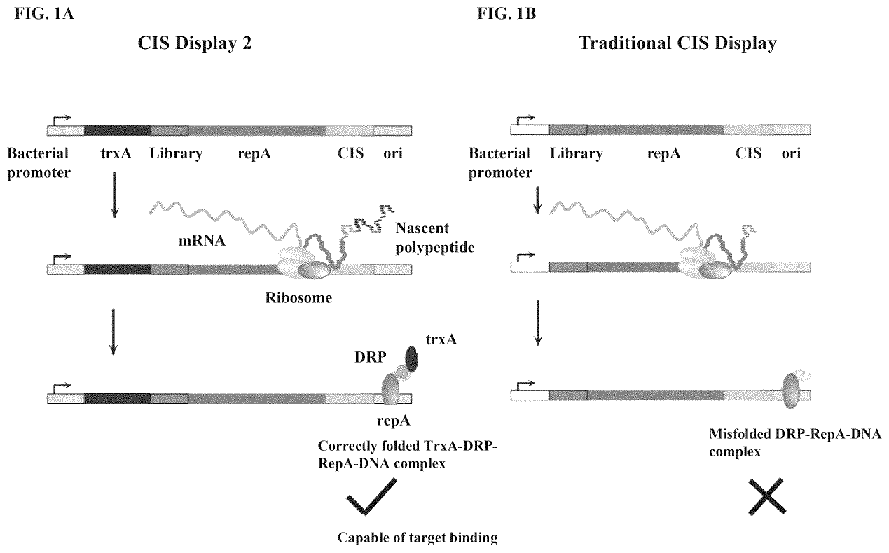

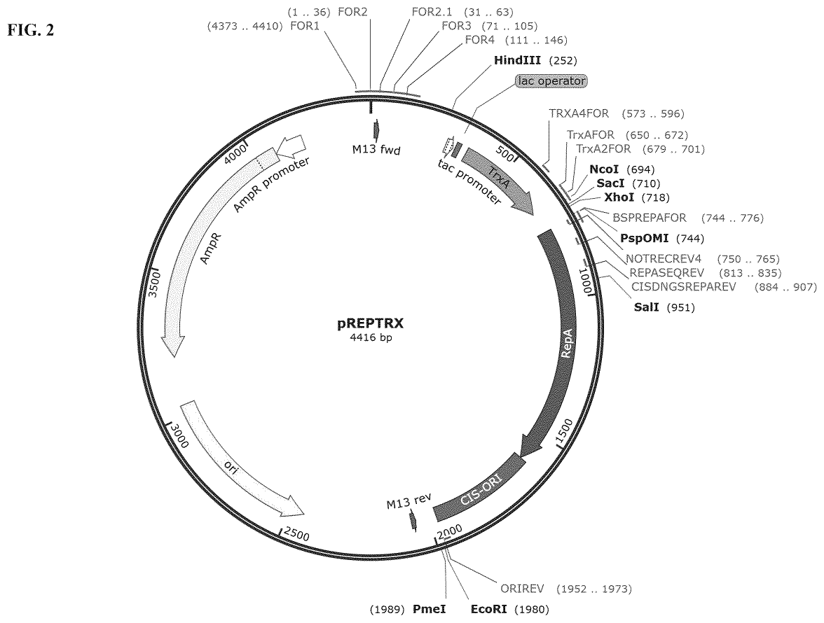

In particular, available methods of producing DRPs and screening for binding ligands are not entirely satisfactory. Existing CIS display libraries (FIG. 1B) are applicable for short linear proteins and are less compatible with DRPs, which include two or more disulfide bonds. Such methods are not easily amenable to the production of functional and correctly folded DRPs as the CIS display lysate is not an environment where disulfide bonds can be efficiently produced. Further the use of methods lacking the ability to produce correctly folded DRPs may compromise the integrity and functionality of the displayed and screened DRP. Other cell-free display methods, such as ribosome display, also have been unsuitable for display of DRPs.

Disulfide-rich proteins (DRPs) are promising binding molecules for drug discovery and for further development. However, the ability to correctly fold DRPs is required for the successful screening of DRPs and for the assessment of DRPs binding to molecular targets of interest. Additionally, in the cell, proteins that contain disulfide bonds are found primarily in relatively oxidizing environments. Hence, the formation of disulfide bonds in reducing environments can be challenging. Provided herein are methods to produce functional DRPs and to screen said proteins in a high-throughput manner.

Remarkably, it is found herein that a disulfide rich protein can be produced and screened using a CIS display method comprising one or more molecular chaperones (FIG. 1A). In the improved method, the libraries are created by the fusion of natural or synthetic libraries of disulfide rich proteins and microproteins (collectively referred to as DRPs) with the one or more molecular chaperones. Overall, the library of DNA constructs undergoes a coupled in vitro transcription-translation reaction (ITT) in a bacterial (e.g., E. coli) lysate, to express a protein fusion containing the DRP and replication initiator protein, which optionally also can include the molecular chaperone (DRP-chaperone-replication initiator protein fusion protein) and link the expressed protein to the DNA fragment from which it was transcribed and translated from (e.g., the DNA:protein fusion protein). The bacterial lysate is a reducing environment. Typically, the reducing environment prevents the correct formation of disulfide bonds, resulting in unstable and aggregated disulfide-rich proteins such as antibodies, cyclotides, and cow VH CDR3 knobs. The inclusion of one or more molecular chaperones in the methods provided herein allows for more diverse protein libraries to be screened, as the one or more molecular chaperones allow for proper folding, functionality, and display of the disulfide rich proteins.

The DRPs can be derived from natural or synthetic libraries. Non-limiting examples of DRPs that can be produced and screened by the provided method include antibodies, scFv fragments, cyclotides, knottins, toxins, bovine CDR3 knob domain, such as cow CDR3 knob domains, amongst others disulfide rich proteins. The chaperones can include trxA, DsbA, DsbC, or other molecular chaperones. In some embodiments, the DRP is fused at the N- and/or C-terminus of the chaperone and which is operably linked to the N-terminus of the CIS Display RepA protein. Using the methods provided herein, the DRPs are correctly expressed and produced as functional binding units that retain binding affinity against targets of interest, e.g., SARS-CoV2. This represents a substantial improvement over standard CIS Display as, surprisingly, it allows proper folding and display of disulfide rich proteins (two or more disulfide bonds) as fusions with the replication initiator protein (e.g., RepA) and subsequent selection of proteins bound to binding ligands of interest. The differences between traditional CIS Display and the current and improved methods are shown in FIG. 1A and FIG. 1B, and several examples of the improved CIS Display technology are provided herein.

Provided herein are methods of producing disulfide rich proteins (DRPs) CIS display libraries and screening said libraries for binding of molecules of interest. Provided herein are methods of screening in vitro DRP expression libraries. Both natural or synthetic libraries of disulfide rich proteins and microproteins can be screened. DRPs comprise antibodies, scFv fragments, cyclotides, knottins, toxins, and bovine CDR3 knob domains, such as cow CDR3 knob domains. Further, proteins comprising disulfide-rich domains whose global folds are stabilized primarily by the formation of disulfide bonds can be used in the generation of CIS display libraries and screened. Disulfide-rich domains perform a wide variety of roles functioning as growth factors, toxins, enzyme inhibitors, hormones, pheromones, allergens, etc. These domains are commonly found both as independent (single domain) proteins and as domains within larger polypeptides. Also provided herein are methods to produce functional DRPs in vitro.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the articles “a” and “an” refer to one or to more than one (i.e. to at least one) of the grammatical object of the article. By way of example, “an element” means one element or more than one element.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

As used herein, the term “about” will be understood by persons of ordinary skill in the art and will vary to some extent on the context in which it is used. As used herein, “about” when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

A “disulfide rich protein” or “disulfide rich polypeptides” or “disulfide rich peptides” or “DRP” or “DRPs”, used interchangeably herein, include peptides or proteins that are constrained through two or more disulfide bonds. DRPs can be derived from natural or synthetic sources. Non-limiting examples of DRPs include antibodies, scFv fragments, cyclotides, knottins, toxins, cow CDR3 knob domains, amongst others disulfide rich proteins. A DRP may be a peptide or protein sequence of 15-300 amino acids in length or longer. In some of any embodiments, the DRP is at least 42 amino acids in length. In some of any embodiments, the DRP is 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, 55 amino acids, 56 amino acids, 57 amino acids, 58 amino acids, 59 amino acids, or 60 amino acids in length. In some of any embodiments, the DRP is at least 65 amino acids, 70 amino acids, 75 amino acids, 80 amino acids, 85 amino acids, 90 amino acids, 95 amino acids, 100 amino acids, 105 amino acids, 110 amino acids, 120 amino acids, 130 amino acids, 140 amino acids, 150 amino acids, 160 amino acids, 170 amino acids, 180 amino acids, 190 amino acids, 200 amino acids, 225 amino acids, 250 amino acids, 275 amino acids, or at least 300 amino acids in length. In some of any embodiments, the DRP comprises at least four cysteine residues. In some of any embodiments, the DRP contains four cysteine residues. In some of any embodiments, the DRP contains 6, 8, 10, or 12 cysteine residues. In some of any embodiments, the DRP has at least two disulfide bonds. In some of any embodiments, the DRP has two disulfide bonds. In some of any embodiments, the DRP has 3, 4, 5, 6, 7, or 8 disulfide bonds.

The terms “DRP-chaperone-replication initiator protein fusion”, “DRP-chaperone-replication initiator protein”, “DRP-chaperone-replication initiator protein DNA”, “chaperone-DRP-replication initiator protein fusion”, “DRP-chaperone-RepA”, and “chaperone-DRP-RepA” refer to a molecule comprising a DRP, one or more molecular chaperones, and a replication initiator protein with cis-activity. This fusion can be found in the context of DNA, RNA, protein, or a DNA:protein fusion. For example, in the DNA construct or vector this molecule is entirely DNA. In another example, transcription of the DNA construct will produce this termed molecule in an RNA context. In yet another example, translation of the transcribed product will produce this termed molecule in a protein context. Lastly, binding of the protein with cis-activity to the DNA construct will produce this termed molecule in the DNA:protein fusion context. The order of the termed molecule does not indicate that that is the order in which the elements are arranged in the actual molecular context.

An “ultralong CDR3” or an “ultralong CDR3 sequence”, used interchangeably herein, comprises a CDR3 or CDR3 sequence that is not derived from a human antibody sequence. An ultralong CDR3 may be 35 amino acids in length or longer, for example, 40 amino acids in length or longer, 45 amino acids in length or longer, 50 amino acids in length or longer, 55 amino acids in length or longer, or 60 amino acids in length or longer. In some embodiments, the ultralong CDR3 is 25-70 amino acids in length, such as 40-70 amino acids in length. Typically, the ultralong CDR3 is a heavy chain CDR3 (CDR-H3 or CDRH3). An ultralong CDR3H3 exhibits features of a CDRH3 of a ruminant (e.g., bovine) sequence. The structure of an ultralong CDR3 includes a “stalk”, composed of ascending and descending strands (e.g., each about 12 amino acids in length), and a disulfide-rich “knob” that sits atop the stalk. The unique “stalk and knob” structure of the ultralong CDR3 results in the two antiparallel β-strands (an ascending and descending stalk strand) supporting a disulfide bonded knob protruding out of the antibody surface to form a mini antigen binding domain. In some embodiments, the ultralong CDR3 antibodies comprise, in order, an ascending stalk region, a knob region, and a descending stalk region.

As used herein, a “CDR3-knob” or “knob,” which are used interchangeably refers to a portion of an ultralong CDR3 that is a peptide sequence typically of 20-70 amino acids in length and that has 2-12 cysteine residues capable of forming 1-6 intramolecular disulfide bonds. For example, a CDR3-knob refers to a portion of an ultralong CDR3 that is a peptide sequence of 40-70 amino acids in length, where said CDR3-knob has at least 4 non-canonical Cys residues, such as 6, 8, 10 or up to 12 non-canonical cysteine residues, and forms 2-6 disulfide bonds. Typically, a knob contains an initial cysteine residue with the amino acid motif cysteine-proline (CP). In some cases, a CDR3-knob may be positioned between an ascending stalk (Stalk A) or a descending stalk (Stalk B) in an antibody or antigen-binding fragment containing the ultralong CDR3, in which the CDR3-knob protrudes out of the antibody interface to form an antigen binding site with an antigen. In other cases, a CDR3-knob may be independently produced as a “knob” peptide as described herein.

As used herein, a “knob peptide”, “CDR3-knob peptide” or “knob-only peptide,” which are terms used interchangeably, refers to an independently produced linear disulfide-bonded peptide that is typically 20-70 amino acids in length and that has 2-12 cysteine residues capable of forming 1-6 intramolecular disulfide bonds. For example, a CDR3 knob peptide refers to an independently produced linear disulfide-bonded peptide of 40-70 amino acids in length, and contains 2-6 disulfide bonds formed by at least 4 non-canonical Cys residues, such as 6, 8, 10 or up to 12 non-canonical cysteine residues. A knob peptide may be derived from an ultralong CDR3 or can be produced synthetically. Typically, the first cysteine of the peptide sequences contains an initial cysteine residue with the amino acid motif cysteine-proline (CP). A knob peptide is a linear molecular that is not able to undergo cyclization to form a cyclic molecule.

“Substantially similar,” or “substantially the same”, refers to a sufficiently high degree of similarity between two numeric values (generally one associated with an antibody disclosed herein and the other associated with a reference/comparator antibody) such that one of skill in the art would consider the difference between the two values to be of little or no biological and/or statistical significance within the context of the biological characteristic measured by said values (e.g., K_{d} values). The difference between said two values is preferably less than about 50%, preferably less than about 40%, preferably less than about 30%, preferably less than about 20%, preferably less than about 10% as a function of the value for the reference/comparator antibody.

“Binding affinity” generally refers to the strength of the sum total of noncovalent interactions between a single binding site of a molecule (e.g., an antibody) and its binding partner (e.g., an antigen). Unless indicated otherwise, “binding affinity” refers to intrinsic binding affinity which reflects a 1:1 interaction between members of a binding pair (e.g., antibody and antigen). The affinity of a molecule X for its partner Y can generally be represented by the dissociation constant. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure.

“Percent (%) amino acid sequence identity” with respect to a peptide or polypeptide sequence refers to the percentage of amino acid residues in a candidate sequence that are identical with the amino acid residues in the specific peptide or polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MegAlign (DNASTAR) software. Those skilled in the art can determine appropriate parameters for measuring alignment, including any algorithms needed to achieve maximal alignment over the full length of the sequences being compared.

“Polypeptide,” “peptide,” “protein,” and “protein fragment” may be used interchangeably to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymers.

“Amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. Amino acid analogs refers to compounds that have the same basic chemical structure as a naturally occurring amino acid, e.g., an alpha carbon that is bound to a hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

“Conservatively modified variants” applies to both amino acid and nucleic acid sequences. “Amino acid variants” refers to amino acid sequences. With respect to particular nucleic acid sequences, conservatively modified variants refer to those nucleic acids which encode identical or essentially identical amino acid sequences, or where the nucleic acid does not encode an amino acid sequence, to essentially identical or associated (e.g., naturally contiguous) sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, silent variations of a nucleic acid which encodes a polypeptide is implicit in a described sequence with respect to the expression product, but not with respect to actual probe sequences. As to amino acid sequences, one of skill will recognize that individual substitutions, deletions or additions to a nucleic acid, peptide, polypeptide, or protein sequence which alters, adds, or deletes a single amino acid or a small percentage of amino acids in the encoded sequence is a “conservatively modified variant” including where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles disclosed herein. Typically conservative substitutions include: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine(S), Threonine (T); and 8) Cysteine (C), Methionine (M) (see, e.g., Creighton, Proteins (1984)).

“Humanized” or “Human engineered” forms of non-human (e.g., bovine) antibodies are chimeric antibodies that contain amino acids represented in human immunoglobulin sequences, including, for example, wherein minimal sequence is derived from non-human immunoglobulin. For example, humanized or human engineered antibodies may be non-human (e.g., bovine) antibodies in which some residues are substituted by residues from analogous sites in human antibodies (see, e.g., U.S. Pat. No. 5,766,886). A humanized antibody optionally may also comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992). See also the following review articles and references cited therein: Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994).

A “variable domain” with reference to an antibody refers to a specific Ig domain of an antibody heavy or light chain that contains a sequence of amino acids that varies among different antibodies. Each light chain and each heavy chain have one variable region domain (VL, and VH). The variable domains provide antigen specificity, and thus are responsible for antigen recognition. Each variable region contains CDRs that are part of the antigen binding site domain and framework regions (FRs).

A “constant region domain” refers to a domain in an antibody heavy or light chain that contains a sequence of amino acids that is comparatively more conserved among antibodies than the variable region domain. Each light chain has a single light chain constant region (CL) domain, and each heavy chain contains one or more heavy chain constant region (CH) domains, which include, CH1, CH2, CH3 and, in some cases, CH4. Full-length IgA, IgD and IgG isotypes contain CH1, CH2 CH3 and a hinge region, while IgE and IgM contain CH1, CH2 CH3 and CH4. CH1 and CL domains extend the Fab arm of the antibody molecule, thus contributing to the interaction with antigen and rotation of the antibody arms. Antibody constant regions can serve effector functions, such as, but not limited to, clearance of antigens, pathogens, and toxins to which the antibody specifically binds, e.g., through interactions with various cells, biomolecules and tissues.

An antibody containing an ultralong CDR3 is an antibody that contains a variable heavy (VH) chain with an ultralong CDR3. An antibody may further include pairing of the VH chain with a variable light (VL) chain. In some embodiments, the antibodies or antigen-binding fragments include a heavy chain variable region and a light chain variable region. Thus, the term antibody includes full-length antibodies and portions thereof including antibody fragments, wherein such contain a heavy chain or portion thereof and/or a light chain or portion thereof. An antibody can contain two heavy chains (which can be denoted H and H′) and two light chains (which can be denoted L and L′), in which each L chain is linked to an H chain by a covalent disulfide bond and the two H chains are linked to each other by disulfide bonds. The terms “full-length antibody,” or “intact antibody” are used interchangeably to refer to an antibody in its substantially intact form, as opposed to an antibody fragment. A full-length antibody is an antibody typically having two full-length heavy chains (e.g., VH-CH1-CH2-CH3 or VH-CH1-CH2-CH3-CH4) and two full-length light chains (VL-CL) and hinge regions.

The term “antibody” herein is used in the broadest sense and includes polyclonal and monoclonal antibodies, including intact antibodies and functional (antigen-binding) antibody fragments, including fragment antigen binding (Fab) fragments, F(ab′)₂ fragments, Fab′ fragments, Fv fragments, recombinant IgG (rIgG) fragments, heavy chain variable (V_{H}) regions capable of specifically binding, and single chain variable fragments (scFv).

An “antibody fragment” comprises a portion of an intact antibody, the antigen binding and/or the variable region of the intact antibody. Antibody fragments, include, but are not limited to, Fab fragments, Fab′ fragments, F(ab′)₂ fragments, Fv fragments, disulfide-linked Fvs (dsFv), Fd fragments, Fd′ fragments; single-chain antibody molecules, including single-chain Fvs (scFv) or single-chain Fabs (scFab); antigen-binding fragments of any of the above and multispecific antibodies from antibody fragments.

A “Fab fragment” is an antibody fragment that results from digestion of a full-length immunoglobulin with papain, or a fragment having the same structure that is produced synthetically, e.g., by recombinant methods. A Fab fragment contains a light chain (containing a V_{L} and C_{L}) and another chain containing a variable domain of a heavy chain (V_{H}) and one constant region domain of the heavy chain (C_{H}1).

An “scFv fragment” refers to an antibody fragment that contains a variable light chain (V_{L}) and variable heavy chain (V_{H}), covalently connected by a polypeptide linker in any order. The linker is of a length such that the two variable domains are bridged without substantial interference. Exemplary linkers are (Gly-Ser)ₙ residues with some Glu or Lys residues dispersed throughout to increase solubility.

The term, “corresponding to” with reference to positions of a protein, such as recitation that nucleotides or amino acid positions “correspond to” nucleotides or amino acid positions in a disclosed sequence, such as set forth in the Sequence listing, refers to nucleotides or amino acid positions identified upon alignment with the disclosed sequence based on structural sequence alignment or using a standard alignment algorithm, such as the GAP algorithm. For example, corresponding residues of a similar sequence (e.g., fragment or species variant) can be determined by alignment to a reference sequence by structural alignment methods. By aligning the sequences, one skilled in the art can identify corresponding residues, for example, using conserved and identical amino acid residues as guides.

The term “effective amount” or “therapeutically effective amount” as used herein means an amount of a pharmaceutical composition which is sufficient enough to significantly and positively modify the symptoms and/or conditions to be treated (e.g., provide a positive clinical response). The effective amount of an active ingredient for use in a pharmaceutical composition will vary with the particular condition being treated, the severity of the condition, the duration of treatment, the nature of concurrent therapy, the particular active ingredient(s) being employed, the particular pharmaceutically-acceptable excipient(s) and/or carrier(s) utilized, and like factors with the knowledge and expertise of the attending physician.

As used herein, the term “pharmaceutically acceptable” refers to a material, such as a carrier or diluent, which does not abrogate the biological activity or properties of the compound, and is relatively nontoxic, i.e., the material may be administered to an individual without causing undesirable biological effects or interacting in a deleterious manner with any of the components of the composition in which it is contained.

As used herein, a composition refers to any mixture of two or more products, substances, or compounds, including cells. It may be a solution, a suspension, liquid, powder, a paste, aqueous, non-aqueous or any combination thereof.

As used herein, the term “pharmaceutical composition” refers to a mixture of at least one compound of the invention with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to, intravenous, oral, aerosol, parenteral, ophthalmic, pulmonary, and topical administration and administration via inhalation.

As used herein, “disease or disorder” refers to a pathological condition in an organism resulting from cause or condition including, but not limited to, infections, acquired conditions, genetic conditions, and characterized by identifiable symptoms.

As used herein, the terms “treat,” “treating,” or “treatment” refer to ameliorating a disease or disorder, e.g., slowing or arresting or reducing the development of the disease or disorder, e.g., a root cause of the disorder or at least one of the clinical symptoms thereof.

As used herein, the term “subject” refers to an animal, including a mammal, such as a human being. The term subject and patient can be used interchangeably.

As used herein, “optional” or “optionally” means that the subsequently described event or circumstance does or does not occur, and that the description includes instances where said event or circumstance occurs and instances where it does not. For example, an optionally substituted group means that the group is unsubstituted or is substituted.

### II. Cis Display Libraries and Method of Generating Same

Provided herein in some aspects are CIS display libraries and methods of producing and preparing CIS display libraries for screening of disulfide rich proteins (DRPs). Also provided herein in some aspects are methods of producing and screening DNA libraries of DRPs, for instance to identify DRPs with increased binding affinity for a target molecule. In some aspects, the DRP CIS display libraries encode disulfide rich proteins (DRPs).

In provided embodiments, the methods described herein employ a cell-free protein synthesis system that is a reduced acellular environment, such as derived from prokaryotic extracts (e.g., E. coli) that is inherently reducing in redox potential. The prokaryotic extract thus acts as a cell-free transcription/translation environment for an in vitro transcription-translation (ITT) because it is an in vitro mimic of the cytosol, but one that is often reducing (due to thioredoxin, glutathione, etc.). This makes it easy to synthesize proteins in bulk, but hard for disulfide-rich proteins to fold properly. In provided embodiments, a prokaryotic cell-free environment, such as provided by an E. Coli extract, retains the molecular machinery required to transcribe DNA into RNA and to translate RNA into protein. Such extracts typically include ribosomes, tRNAs, aminoacyl-tRNA synthetases, translation factors to permit in vitro protein synthesis from added DNA templates. The extracts may be supplemented with appropriate cofactors and energy sources. However, such an environment normally disfavors disulfide bonding formation. In some embodiments, the methods herein thus employ a prokaryotic cell-free transcription/translation environment, in combination with at least one molecular chaperone to improve the folding and stability of the disulfide-rich proteins being expressed. A plurality of DNA constructs are prepared, each construct comprising a DNA sequence encoding a DRP, a DNA sequence encoding a DNA replication initiator protein such as RepA or a functional variant thereof, and a DNA sequence encoding a target sequence for binding by the DNA replication initiator protein. Expression of the plurality of DNA constructs in the cell-free transcription/translation environment leads to the formation of DNA:protein fusion proteins, wherein each DNA replication initiator protein binds its cognate target sequence within the DNA construct from which it was transcribed, thereby linking the encoded DRP to its DNA. The resulting complexes form a CIS display library in which each DRP is associated with its encoding DNA sequence.

In some embodiments, the method of producing a DRP CIS display library involves expressing a plurality of DNA constructs in a prokaryotic cell-free transcription/translation environment in the presence of at least one molecular chaperone, wherein the at least one molecular chaperone is provided by (i) expression from a DNA sequence encoding the molecular chaperone included in the plurality of DNA constructs, or (ii) addition of one or more DNA sequences encoding the molecular chaperone or the molecular chaperone itself to the transcription/translation environment. In such embodiments, each DNA construct comprises a DNA sequence encoding one of a plurality of disulfide rich protein (DRP); a DNA sequence encoding a DNA replication initiator protein; and a DNA sequence encoding a target sequence for the DNA replication. Expressing the plurality of DNA constructs in this way produces a plurality of DNA:protein fusion proteins comprising the DRP and the DNA replication initiator protein and, optionally the molecular chaperone, from the plurality of DNA constructs, wherein each fusion protein binds, via the DNA replication initiator protein, to the target sequence in the DNA construct from which it was transcribed, thereby linking the fusion protein to the DNA construct to produce the DRP CIS display library comprising a plurality of DNA:protein fusion proteins.

In some embodiments, the prokaryotic cell-free transcription/translation environment is prepared or adapted based on methods as described in Lesley, S. A. et al. (1991) J. Biol. Chem. 266, 2632-8. The transcription/translation environment may be a reduced acellular environment, such as an E. coli S30 extract system, a PURE system, or another prokaryotic lysate suitable for coupled transcription/translation and protein folding. Examples are described in Section II.D. An example of a prokaryotic cell-free transcription/translation environment is the E. coli S30 Extract System (Promega, Cat. No. L1030), which allows successful transcription/translation of linear DNA templates. In such systems, the DNA construct typically comprises sequence elements suitable for expression in a bacterial extract, such as a prokaryotic promoter, a ribosome binding site, and a transcriptional terminator, in addition to the coding sequence of the disulfide-rich protein (DRP) to be expressed, a sequence encoding a DNA replication initiator protein such as RepA, and a non-coding target sequence (e.g., an origin of replication sequence) recognized by the initiator protein. In some embodiments, the DNA construct further comprises a sequence encoding a molecular chaperone. In other embodiments, the chaperone is provided separately as an added nucleic acid template or as a purified protein.

In provided embodiments, the methods for producing a DRP CIS display library involve expressing a plurality of DNA constructs in which each DNA construct of the plurality encodes a DRP. In some embodiments, each encoded DRP of the library is different, such that the plurality of DNA constructs collectively encode a diverse set of DRPs that vary in sequence, structure, and/or number and arrangement of cysteine residues. In certain versions, the DRPs may be selected from naturally occurring cysteine-rich scaffolds, engineered variants thereof, or synthetic sequences designed de novo. The number of distinct DRPs in a library may range from thousands to millions, with each DRP sequence represented in at least one DNA construct and expressed in the presence of chaperone activity, thereby enabling the assembly of a robust DRP CIS display library. In some embodiments, the library comprises a large number of distinct DRPs, for example at least 10³, at least 10⁴, at least 10⁵, or at least 10⁶ different DRPs, thereby enabling high-throughput screening and identification of members with desirable properties. The encoded DRPs may include knob peptides derived from or based on knob domain of an ultralong CDR3 antibody, such as cow-derived knob peptides, knottins, cyclotides, defensins, toxin-derived scaffolds, or other proteins or peptides characterized by two or more disulfide bonds. Examples of exemplary DRPs are described in Section II.A.1. In some embodiments, the plurality of DNA constructs are designed to encode DRPs of varying loop lengths, disulfide connectivities, or sequence diversities at one or more randomized positions, thereby providing a combinatorial library suitable for selection using CIS display methods.

The molecular chaperone is provided to the prokaryotic cell-free transcription/translation environment to facilitate the correct folding of the DRPs and the formation of native disulfide bonds. In certain embodiments, the chaperone is encoded within the same DNA construct that encodes the DRP. In this manner, both the DRP and the chaperone are expressed simultaneously, and the chaperone acts in cis to assist the folding of its associated DRP. In some versions, the chaperone is expressed as a separate protein, while in other versions the chaperone is expressed as a fusion protein with the DRP, optionally joined by a peptide linker, that enables intramolecular or intermolecular assistance in folding. In other embodiments, the chaperone is encoded on a separate DNA construct that is co-expressed in the same transcription/translation reaction as the DRP constructs, thereby supplying the chaperone in trans to interact with multiple different DRPs. In yet other embodiments, the chaperone is supplied by direct addition of the purified chaperone protein to the cell-free system, thereby ensuring sufficient concentrations of the chaperone are present during translation and folding. Suitable molecular chaperones include any described in Section II.A.2, and combinations or engineered variants thereof.

In some embodiments, each DNA construct of the library encodes a DRP and a chaperone sequence, such that each resulting DNA:protein fusion complex comprises a DRP, the DNA replication initiator protein, and the chaperone. In some embodiments, the molecular chaperone is provided by expression from a DNA sequence encoding the molecular chaperone included in each of the DNA constructs, and wherein each of the plurality of DNA:protein fusion proteins comprises a DRP, the molecular chaperone, and the DNA replication initiator protein.

In some aspects, the CIS display libraries encode one or more chaperone proteins at the N- and/or C-terminus of the DRP. In some embodiments, the CIS display libraries encode one or more chaperone proteins at the N-terminus of the DRP. In some embodiments, the CIS display libraries encode one or more chaperone proteins at the C-terminus of the DRP. In some embodiments, the method comprises providing a plurality of DNA constructs, wherein each DNA construct comprises a DNA sequence encoding a DRP, one or more DNA sequences encoding a molecular chaperone, a DNA sequence encoding a DNA replication initiator protein, and a DNA sequence encoding a target sequence for the DNA replication initiator protein (e.g., RepA), wherein the DNA replication initiator protein can non-covalently bind to the target sequence. In some embodiments, the DNA construct comprises DNA encoding a library member DRP and means for the encoded DRP to bind to the encoding DNA construct (e.g., RepA, cis, and ori DNA elements). Examples of DNA constructs encoding CIS Display library members are described in Section II.A.

In other embodiments, the chaperone is provided only from one or more separate DNA constructs or from added chaperone protein, such that each fusion complex comprises a DRP and the DNA replication initiator protein, but not necessarily the chaperone. For instance, at least one molecular chaperone is provided by addition of one or more DNA sequences encoding the molecular chaperone or the molecular chaperone itself to the reduced acellular environment. In some embodiments, the method comprises providing a plurality of DNA constructs, wherein each DNA construct comprises a DNA sequence encoding a DRP, a DNA sequence encoding a DNA replication initiator protein, and a DNA sequence encoding a target sequence for the DNA replication initiator protein (e.g., RepA), wherein the DNA replication initiator protein can non-covalently bind to the target sequence, expressing the plurality of DNA constructs in a prokaryotic cell-free transcription/translation environment to produce a plurality of DNA:protein fusion proteins comprising the DRP and the DNA replication initiator protein from the plurality of DNA constructs, and adding a molecular chaperone, or one or more DNA sequences encoding a molecular chaperone, to the prokaryotic cell-free transcription/translation environment, during the expressing the plurality of DNA constructs. In some aspects, addition of the one or more DNA sequences encoding the molecular chaperone or the molecular chaperone itself maintains disulfide bonding to allow expression of the DRP in the prokaryotic cell-free transcription/translation environment (e.g., the reduced acellular environment) without aggregation.

In some embodiments, the DRP sequences are derived from natural or synthetic origins. DRPs sequences encoded by the plurality of DNA constructs are, for example, derived from knob DRPs, synthetic DRPs, cyclotide DRPs, and/or scFv DRPs. In some embodiments, the encoded DRP comprises an antibody, an scFv fragment, a cyclotide, a knottin, a toxin, and a cow CDR3 knob domain. Examples of DNA constructs encoding CIS Display DRP sequences are described in Section II.A.1.

Provided herein in some aspects are DRP CIS display libraries encoding molecular chaperones. In some embodiments, the one or more molecular chaperone is encoded by the DNA construct at the N- and/or C-terminus of the DRP. Also provided herein in some aspects are DRP CIS display libraries without encoding molecular chaperones. In some embodiments, the one or more molecular chaperone is encoded separately and/or is separately provided from the DRP. The molecular chaperone sequence is of bacterial or eukaryotic origin. In some embodiments, the molecular chaperone sequence encodes or is a protein DsbA. In some embodiments, the molecular chaperone sequence encodes or is a protein trxA. Examples of molecular chaperones are described in Section II.A.2.

Further, the DNA construct and the encoded protein have cis-activity. That is, the encoded protein has the ability to bind specifically to the DNA molecule which encoded it. For example, cis-activity may function to allow the encoded DNA replication initiator protein (e.g., RepA) to bind specifically (directly or indirectly) to the target sequence of the DNA construct which encoded it rather than to the target sequence of another DNA construct. A DNA element that directs cis-activity may be provided in the DNA construct together with the DNA encoding a peptide that interacts with that cis element. For example, in the case of the cis element from the repA system, DNA encoding a fragment of the repA sequence comprising at least 20 amino acids from the C terminal of repA may be provided along with the cis DNA element. It may be possible to confer cis activity upon a DNA binding peptide that is not normally cis-acting by including in the DNA construct such a DNA element and any further sequences necessary for its action. For example, DNA encoding a peptide that interacts with the cis element used may be included in the DNA construct. A suitable DNA element may be any element which allows or directs cis-activity. Such a DNA element may act, for example, by interacting with the machinery involved in translation and transcription of the DNA construct to delay the production and release of the encoded peptide. Examples of replication initiator proteins and other cis-activity DNA construct components are described in Section II.A.3.

A CIS display library provided herein therefore encompasses a library of DRPs which are non-covalently associated with the DNA which encoded them. Provided herein in some aspects are a plurality of DNA constructs encoding CIS display libraries. In some embodiments, each member of the plurality of DNA constructs comprises a DNA sequence encoding a different DRP. For example, a CIS display library of the present invention may be a library of at least 10⁴ or at least 10⁶ discrete DNA constructs encoding DRPs as fusion proteins with the replication initiator (e.g., RepA) and, in some embodiments alsoone or more molecular chaperones. Each CIS display library comprising a library of DRP fusion proteins, such as DRP-chaperone sequences, is expressed from a library of DNA molecules or a plurality of DNA constructs. In some embodiments, the encoded DRPs comprises DRP variants comprising one or more differences in their amino acid residues. A CIS display library, such as a DRP-chaperone expression library o of the invention may be any library formed by the expression of a library of DNA constructs of the present invention. Examples of CIS display libraries encoded by a plurality of DNA constructs are described in Section II.B.

Further, methods of generating a CIS display library by in vitro transcription/translation are described herein. In some aspects, expressing the plurality of DNA constructs in a prokaryotic cell-free transcription/translation environment produces a plurality of DNA:protein fusions comprising protein fusions of the DRP and the DNA replication initiator protein (e.g. RepA), and in some embodiments the chaperone, such as a DRP-chaperone-DNA replication initiator proteins, from the plurality of DNA constructs. Expressing such a library of DNA constructs results in the non-covalent binding of individual encoded proteins to the DNA which encoded them and from which they have been transcribed and translated, in the presence of many other DNA molecules that encode other members of the library. In some aspects, the encoded protein fusions, such as DRP-chaperone-DNA replication initiator proteins, are linked to the corresponding DNA construct by binding of the DNA replication initiator protein to the target sequence in the corresponding DNA construct. The sequence encoding the library member present in a particular encoded protein will therefore be present in the DNA which is bound to that protein. This process therefore links the library member in a biologically active form (usually having a binding activity) to the specific library member DNA sequence encoding that library member, allowing selection of DRPs of interest, for example due to a particular binding activity, and subsequent isolation and identification of the DNA encoding that library member DRP. Examples of methods of generating CIS display libraries encoded by a plurality of DNA constructs are described in Section II.C.

An in vitro DRP expression library produced by a method provided herein may be used to screen for particular members of the library. For example, the library may be screened for DRPs with a particular activity or a particular binding affinity. Protein-DNA complexes of interest may be selected from a library by, for example, affinity or activity enrichment techniques. This can be accomplished by means of a ligand specific for the protein of interest, such as an antigen if the protein of interest is an antibody. The ligand may be presented on a solid surface such as the surface of an ELISA plate well, or in solution, for example, with biotinylated ligand followed by capture onto a streptavidin coated surface or magnetic beads, after a library of protein-DNA complexes had been incubated with the ligand to allow ligand-ligand interaction. Following either solid phase or in solution incubation, unbound complexes are removed by washing, and bound complexes isolated by disrupting ligand-ligand interactions by altering pH in the well, or by other methods known to those skilled in the art such as protease digestion, or by releasing the DNA directly from the complexes by heating or phenol-chloroform extraction to denature the repA-ori DNA binding. DNA can also be released by one of the methods above, directly into PCR buffer, and amplified. Alternatively, DNA may be PCR amplified directly without release from the complexes. Examples of methods of screening produced CIS display libraries and target selection are described in Section II.D.

Also provided herein in some embodiments are methods of producing soluble disulfide rich peptides or proteins identified by the screen. Soluble peptides produced according to the provided methods include CDR3 knobs, scFV, cyclotides, semi-synthetic or synthetic DRPs, and other DRPs. Examples of expressing soluble DRPs of interest are described in Section II.E.

In some of any embodiments, the method of generating a CIS display library providing a plurality of DNA constructs that are prepared by ligation of a sequence encoding a DRP from a library of DRPs in frame with the replication initiator protein (e.g., RepA), and in optionally one or more molecular chaperone, and creating a single expression cassette encoding a DRP-RepA. In some of any embodiments, the method of generating a CIS display library involves ligation of the DNA construct encoding a DRP or a library of DRPs in frame with the one or more molecular chaperone and the replication initiator protein (e.g., RepA), creating a single expression cassette of encoding a chaperone-DRP-RepA. The expression cassette is designed to be under control of a promoter to drive the translation reaction. In some embodiments, the DNA construct, such as a DNA construct containing a promoter-chaperone-DRP-RepA-CIS-ORI DNA construct or promoter-DRP-RepA-CIS-ORI DNA, is amplified. In some embodiments, a coupled in vitro transcription-translation (ITT) reaction is performed to express the fusion protein, such as the chaperone-DRP-RepA fusion protein. In some embodiments, this ITT expression links the expressed protein to the DNA fragment from which it was transcribed and translated from. In some embodiments, once generated, the linked DNA:protein fusion, such as chaperone-DRP-RepA DNA:protein complex, or a library of such complexes, are contacted with one or more targets of interest. In some embodiments, a linked DNA:protein complexes bind to targets of interests. In some embodiments, the non-bound DNA:protein complexes are washed away and the bound DNA:protein complexes are recovered. In some embodiments, the DNA of the bound DNA:protein complexes is recovered encoding the binding DRP. In some embodiments, one, two, three, four, or up to ten further rounds of selection may be carried out to enrich for DRPs binding to a target of interest. In some embodiments, the enrichment is done by reconstituting the DNA construct, such as the promoter-chaperone-DRP-RepA-CIS-ORI DNA cassette, and repeating the ITT reaction and the binding step. In some embodiments, the enriched DRPs are identified by nucleic acid sequencing. In some embodiments, the sequenced enriched DRPs are cloned into a suitable expression system for expression of individual sequences. In some embodiments, the individual sequences are expressed and binding of the target of interest is measured. In some embodiments, expression systems include pIII phage display, TrxA (SEQ ID NO: 11) or DsbA (SEQ ID NO: 12) fusion in bacteria, antibody Fc, or other fusion and screening in mammalian cell culture.

### A. DNA Constructs Encoding CIS Display Disulfide Rich Proteins

Provided in the methods herein, CIS display library members, each of which encodes a disulfide-rich protein (DRP) to be expressed in the DRP expression library (library member DRP), are placed in suitable DNA constructs (e.g., polynucleotide) that can be used to prepare and produce a CIS display library. CIS display libraries are generated by a method that comprises providing a plurality of DNA constructs, of which each encodes a DRP and, in some embodiments also one or more molecular chaperones, and all the sequences necessary to allow expression of the library member DRP and of the one or more molecular chaperones from the construct. Importantly, the individual DNA library members encode all the sequences necessary to allow the protein fusion encoded by the individual DNA construct to bind to the DNA construct which encoded it. In some embodiments, the constructs encode a fusion of DRP, one or more molecular chaperones, and a replication initiator protein (e.g., RepA). This is collectively referred to as DRP-chaperone-replication initiator protein fusion or complex (e.g., DRP-chaperone-RepA). In other embodiments the chaperone is added separately during the cell-free transcription/translation expression reaction as either a nucleic acid or protein, and the constructs encode a fusion of DRP and a replication initiator protein (e.g., RepA). This is collectively referred to as DRP-replication initiator protein fusion or complex (e.g., DRP-RepA). This fusion can be either DNA, RNA, protein, or a DNA:protein fusion. Such fusion proteins may comprise further sequences and said library DRP may be joined to said replication initiator protein via a linker sequence.

Further, the DNA constructs include appropriate promoter and translation sequences for in vitro transcription/translation. In some embodiments, at the DNA level, the construct is a vector or linear DNA fragment comprising a bacterial promoter such as the tac promoter, ribosome binding site and start codon, the chaperone gene, a linker sequence, the disulfide rich protein encoding gene (or a library of such gene fragments), and second linker, and the RepA coding sequence followed by the CIS and ORI non-coding DNA elements required for Cis activity (CIS) and the DNA binding site (ORI) for the expressed RepA fusion protein. In other embodiments, at the DNA level, the construct is a vector or linear DNA fragment comprising a bacterial promoter such as the tac promoter, ribosome binding site and start codon, the disulfide rich protein encoding gene (or a library of such gene fragments), and a linker, and the RepA coding sequence followed by the CIS and ORI non-coding DNA elements required for Cis activity (CIS) and the DNA binding site (ORI) for the expressed RepA fusion protein. Any suitable promoter can be used, such as the ara B, tac promoter, T7, T3, or SP6 promoters amongst others. The promoter is placed so that it is operably linked to the DNA sequences of the invention such that such sequences are expressed.

Among embodiments provided herein are methods that comprise individual DNA library members, each of which encodes a disulfide rich protein or peptide and one or more molecular chaperones to be expressed in the CIS display library, which are placed in a suitable DNA construct. The DNA construct into which the DNA library member (e.g., DRP-chaperone) is placed includes all the sequences necessary to allow expression of the library member DRP-chaperone from the construct. Importantly, the construct includes the sequences (e.g., DNA element) necessary to allow the peptide encoded by the construct to be associated with the DNA construct which encoded it. A suitable DNA element may be any element which allows or directs cis-activity. Such a DNA element may act, for example, by interacting with the machinery involved in translation and transcription of the DNA construct to delay the production and release of the encoded peptide. For example, each DRP in the library will typically comprise a fusion protein comprising the library member DRP, and one or more chaperones fused to a peptide involved in binding of the fusion protein to the relevant DNA construct (FIG. 1A). Such fusion proteins may comprise further sequences and said library DRP may be joined to said binding peptide via a linker sequence.

The DNA comprising the DNA constructs may be produced by any means. In particular, such DNA may comprise DNA isolated from cDNA, obtained by DNA shuffling, and/or synthetic DNA.

In some embodiments, a DNA construct encoding a CIS display DRP is produced by fusion of a DRP to one or more chaperone as described herein, such as a CDR3 knob peptide and a trxA chaperone fusion.

In some embodiments, a nucleic acid encoding a candidate binding DRP as described herein, such as a CDR3 knob peptide, is inserted into or constructed as part of a DNA construct, in which the nucleic acid is fused to a nucleic acid encoding at least a portion of molecular chaperone. In some embodiments, the nucleic acid encoding a candidate binding DRP, and one or more molecular chaperone as described herein, such as a CDR3 knob protein and trxA, is fused to a replication initiator protein (e.g., RepA).

In some embodiments, ligation of the gene encoding a DRP is ligated in-frame with the molecular chaperone and replication initiator protein (e.g., RepA), creating a single expression cassette of chaperone-DRP-RepA, and, optionally, amplifying the promoter-chaperone-DRP-RepA-CIS-ORI DNA cassette.

The order (e.g., 5′-3′) of the one or more molecular chaperones and the DRP can differ when ligating the expression cassette or cassettes. In some embodiments, the molecular chaperone will be upstream of the DRP. In some embodiments, the molecular chaperone will be downstream of the DRP. In some embodiments, more than one molecular chaperone will be upstream of the DRP. In some embodiments, more than one molecular chaperone will be downstream of the DRP. In some embodiments, two molecular chaperones will be upstream of the DRP. In some embodiments, two molecular chaperones will be downstream of the DRP. In some embodiments, one or more chaperones will be upstream of the DRP, and one or more chaperones will be downstream of the DRP. In some embodiments, one chaperone will be upstream of the DRP, and one chaperone will be downstream of the DRP. In some embodiments, two chaperones will be upstream of the DRP, and two chaperones will be downstream of the DRP. In some of any of the preceding embodiments, the DRP and the one or more chaperones encoded in the N- and/or C-terminus will be in-frame. The individual candidate DNA construct encoding protein fusions, such as DRP-chaperone-RepA, is then ready for in vitro transcription and translation.

### 1. Disulfide Rich Proteins (DRPs)

In some embodiments, the DRP is any protein containing two or more disulfide bonds. As described herein, the provided display library can be formed by the fusion of any disulfide rich protein to a molecular chaperone at the N- or C-terminus, which is operably linked to the RepA protein. In some embodiments, the DRP is a synthetic or semisynthetic disulfide rich protein. Exemplary DRPs include, but are not limited to, antibodies, scFv fragments, cyclotides, knottins, toxins, and cow CDR3 knob domains. In some embodiments, the DRP is a scFv. In some embodiments, the DRP is a cyclotide. In some embodiments, the DRP is a semisynthetic or modified ultralong CDR3 knob. In some embodiments, the modified cyclotide includes an ultralong CDR3 knob sequence, e.g., of a cow. In some embodiments, the DRP is a cow CDR3 knob domain which is a cystine-motif peptide derived from the knob region from the ultralong CDR-H3 of a bovine or bovine-derived antibody.

In some embodiments, the DRP is a cysteine-motif peptide of at least 20 amino acids in length in which 2-12 amino acids are cysteine residues able to form 1-6 disulfide bonds. In some embodiments, any of such peptide is 20-70 amino acids in length, such as 25-70 amino acids or 40-70 amino acids in length, in which 2-12 amino acids are cysteine residues able to form 1-6 disulfide bonds.

In some embodiments, the polypeptides for display contain a variable heavy region containing the ultralong CDR-H3 and a variable light region. Particular formats include single chain formats, such as a single chain variable fragment (scFv). In other embodiments, the polypeptide for display is a smaller peptide of 25-70 amino acids, such as 40-70 amino acids, that is a knob peptide. Exemplary DRP molecules for display and display libraries are described.

The application and screening of DRPs require the correct pairing and formation of disulfide bonds to stabilize the DRPs and produce correctly folded and functional proteins. In some embodiments, the encoded DRP has four or more cysteine residues for forming two or more disulfide bonds. Not only are disulfide bridges often vital for the stability of a final protein structure, but the incorrect pairing of cysteine residues also usually prevents the folding of a protein into its native conformation.

Disulfide bonds are highly conserved across proteomes. Disulfide bonds are formed by the oxidation of two cysteine residues and stabilize proteins by maintaining their overall structure. The formation of a disulfide bond from two thiols (—SH) is a two-electron reaction that requires an oxidant or electron acceptor. Disulfide bonds can be formed spontaneously in vitro by the loss of electrons from two cysteines coupled with the gain of electrons by an available acceptor, such as molecular oxygen. When molecular oxygen is used as an electron acceptor, an intermediary, such as a transition metal or flavin is required. In cells, the formation of structural disulfide bonds primarily occurs in the endoplasmic reticulum (ER) of eukaryotic cells and the periplasmic space of prokaryotic cells. The protein oxidation in the ER relies on the membrane-associated proteins Ero1 (ER oxidoreductin) and Erv2, and the soluble thiol-disulfide oxidoreductase protein disulfide isomerase (PDI). The prokaryotic protein oxidation system uses the integral membrane protein DsbB and the soluble enzyme DsbA. In addition to the DsbA-DsbB pathway for disulfide-bond formation, prokaryotes also contain a pathway for the isomerization of non-native disulfide bonds. This pathway includes the membrane protein DsbD and the soluble enzyme DsbC.

DRPs comprise cysteine motifs. In some embodiments, the cysteine motif includes 2-20 cysteine residues, for instance between or between about 2 and 18, 2 and 16, 2 and 14, 2 and 12, 2 and 10, 2 and 8, 2 and 6, 2 and 4, 4 and 20, 4 and 18, 4 and 16, 4 and 14, 4 and 12, 4 and 10, 4 and 8, 4 and 6, 6 and 20, 6 and 18, 6 and 16, 6 and 14, 6 and 12, 6 and 10, 6 and 8, 8 and 20, 8 and 18, 8 and 16, 8 and 14, 8 and 12, 8 and 10, 10 and 20, 10 and 18, 10 and 16, 10 and 14, 10 and 12, 12 and 20, 12 and 18, 12 and 16, 12 and 14, 14 and 20, 14 and 18, 14 and 16, 16 and 20, 16 and 18, or 18 and 20 cysteine residues, each inclusive. In some embodiments, the cysteine motif includes 2-12 cysteine residues.

DRPs comprise two or more disulfide bonds. In some embodiments, the DRPs include 2-10 disulfide bonds, for instance between or between about 2 and 9, 2 and 8, 2 and 7, 2 and 6, 2 and 5, 2 and 4, 2 and 3, 2 and 2, 2 and 10, 2 and 9, 2 and 8, 2 and 7, 2 and 6, 2 and 5, 2 and 4, 2 and 3, 3 and 10, 3 and 9, 3 and 8, 3 and 7, 3 and 6, 3 and 5, 3 and 4, 4 and 10, 4 and 9, 4 and 8, 4 and 7, 4 and 6, 4 and 5, 5 and 10, 5 and 9, 5 and 8, 5 and 7, 5 and 6, 6 and 10, 6 and 9, 6 and 8, 6 and 7, 7 and 10, 7 and 9, 7 and 8, 8 and 10, 8 and 9, or 9 and 10 disulfide bonds, each inclusive. In some embodiments, the DRPs include 2-6 disulfide bonds. In some embodiments, the soluble peptide contains 2-6 disulfide bonds. In some embodiments, the soluble peptide has at least 2 disulfide bonds. In some embodiments, the soluble peptide has 2 disulfide bonds. In some embodiments, the soluble peptide has 3, 4, or 5 disulfide bonds.

In some embodiments, the DRP is up to 300 amino acids in length. In some of any embodiments, the DRP is at least 42 amino acids in length. In some of any embodiments, the DRP is 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, 55 amino acids, 56 amino acids, 57 amino acids, 58 amino acids, 59 amino acids, or 60 amino acids in length. In some of any embodiments, the DRP is at least 65 amino acids, 70 amino acids, 75 amino acids, 80 amino acids, 85 amino acids, 90 amino acids, 95 amino acids, 100 amino acids, 105 amino acids, 110 amino acids, 120 amino acids, 130 amino acids, 140 amino acids, 150 amino acids, 160 amino acids, 170 amino acids, 180 amino acids, 190 amino acids, 200 amino acids, 225 amino acids, 250 amino acids, 275 amino acids, or at least 300 amino acids in length. In some embodiments, the DRP is 40 to 60 amino acids in length.

In some embodiments, the DRP is 25-70 amino acids. For instance, in some embodiments the DRP is 35 amino acids in length or longer, 40 amino acids in length or longer, 45 amino acids in length or longer, 50 amino acids in length or longer, 55 amino acids in length or longer, or 60 amino acids in length or longer. In some embodiments, the DRP is between or between about 35 and 70 amino acids in length, 40 and 70 amino acids in length, 45 and 70 amino acids in length, 50 and 70 amino acids in length, 55 and 70 amino acids in length, or 60 and 70 amino acids in length. In some embodiments, the DRP is a peptide sequence that is 40, 41, 42, 43, 44, 45, 46, 47, 48, 49, 50, 51, 52, 53, 54, 55, 56, 57, 58, 59, or 60 amino acids in length.

In some embodiments, the DRP is 6 to 50 amino acids, 6 to 40 amino acids, 6 to 30 amino acids, 6 to 25 amino acids, 6 to 20 amino acids, 6 to 15 amino acids, 6 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 amino acids, 25 to 30 amino acids, 30 to 50 amino acids, 30 to 40 amino acids, or 40 to 50 amino acids. In some embodiments, the DRP is 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids.

In some embodiments, the DRP includes a cysteine motif able to form disulfide bonds. In some embodiments, the cysteine motif includes 2-20 cysteine residues, for instance between or between about 2 and 18, 2 and 16, 2 and 14, 2 and 12, 2 and 10, 2 and 8, 2 and 6, 2 and 4, 4 and 20, 4 and 18, 4 and 16, 4 and 14, 4 and 12, 4 and 10, 4 and 8, 4 and 6, 6 and 20, 6 and 18, 6 and 16, 6 and 14, 6 and 12, 6 and 10, 6 and 8, 8 and 20, 8 and 18, 8 and 16, 8 and 14, 8 and 12, 8 and 10, 10 and 20, 10 and 18, 10 and 16, 10 and 14, 10 and 12, 12 and 20, 12 and 18, 12 and 16, 12 and 14, 14 and 20, 14 and 18, 14 and 16, 16 and 20, 16 and 18, or 18 and 20 cysteine residues, each inclusive. In some embodiments, the cysteine motif includes 2-12 cysteine residues. In some embodiments, the DRP comprises at least 4 Cys residues. In some embodiments, the DRP contains 4 Cys residues. In some embodiments, the DRP contains 6, 8, 10, or 12 Cys residues.

In some embodiments, the DRP includes 3-6 amino acids preceding the most N-terminal cysteine residue present in the DRP. In some embodiments, the DRP includes 3, 4, 5, or 6 amino acids preceding the most N-terminal cysteine residue present in the DRP.

In some embodiments, the DRP includes at least 6 amino acids following the most C-terminal cysteine residue present in the DRP. In some embodiments, the DRP includes 6-9 amino acids following the most C-terminal cysteine residue present in the DRP. In some embodiments, the DRP includes 6, 7, 8, or 9 amino acids following the most C-terminal cysteine residue present in the DRP.

CIS display library members comprised of DRPs can include a plurality of DRPs defined by amino acid chains of variable composition of at least two amino acids in length. A suitable DRP library member may include a peptide having a random amino acid composition. The DRP of variable or random composition may be flanked by known amino acid sequences at the N- and/or C-terminus to constrain the structure. These known sequences may vary in length.

In some embodiments, libraries of DRP variants are generated by introducing amino acid sequence differences through mutagenesis of the encoding DNA sequence. The amino acid differences may be produced by random mutagenesis, error-prone PCR, site-directed mutagenesis, or use of degenerate or doped oligonucleotides during gene synthesis. In some embodiments, codons are replaced with degenerate codons (e.g., NNK, NNS, or trimer mixes) to allow sampling of amino acid diversity at one or more positions. In certain embodiments, amino acid differences are introduced by DNA shuffling or recombination of related DRP sequences to generate chimeric variants. In other embodiments, insertions or deletions are introduced to alter loop length, stalk residues, or the number and spacing of cysteine residues, thereby producing DRP variants with distinct disulfide-bonding patterns. Accordingly, a DRP display library may comprise thousands to billions of unique variants, each differing from others in amino acid sequence while retaining a disulfide-constrained fold. In some embodiments the plurality of DRPs comprise at least 10³, at least 10⁵, at least 10⁷, or at least 10⁹ unique members, each member comprising a distinct DRP sequence.

For further details on DRPs see, e.g., Check S, Krishna S S, Grishin N V. Structural classification of small, disulfide-rich protein domains. J Mol Biol. 2006 May 26; 359(1): 215-37. doi: 10.1016/j.jmb.2006.03.017. Epub 2006 Mar. 29. PMID: 16618491; Meng X, Xu C, Fan S, Dong M, Zhuang J, Duan Z, Zhao Y, Wu C. Selection and evolution of disulfide-rich peptides via cellular protein quality control. Chem Sci. 2023 Mar. 15; 14(13): 3668-3675. doi: 10.1039/d2sc05343h. PMID: 37006698; PMCID: PMC10055976; Sevier, C., Kaiser, C. Formation and transfer of disulphide bonds in living cells. Nat Rev Mol Cell Biol 3, 836-847 (2002). doi.org/10.1038/nrm954; Jiang S, Carroll L, Rasmussen L M, Davies M J. Oxidation of protein disulfide bonds by singlet oxygen gives rise to glutathionylated proteins. Redox Biol. 2021 January; 38:101822. doi: 10.1016/j.redox.2020.101822. Epub 2020 Dec. 1. PMID: 33338920; PMCID: PMC7750407, which are incorporated by reference herein in their entireties for all purposes.

a. Knob DRPs

In some embodiments, the disulfide rich protein is a knob DRP. In some embodiments, the DRP comprises a knob DRP. In some embodiments, the knob DRP is or is derived from knob region of an ultralong CDR3 antibody, such as a cow ultralong CDR3 antibody. In some embodiments, the DRP is a CDR3 knobfrom a cow.

Knob domains are compact, disulfide-stabilized peptide regions that are naturally present within the ultralong complementarity-determining region 3 (CDR3) immunoglobulin heavy chains of certain species of the Bovinae subfamily, such as bovine. Unlike conventional CDR3 loops, ultralong CDR3 regions can extend over 50 amino acids and fold into a stalk-and-knob architecture, in which the knob portion adopts an autonomous, cysteine-rich domain stabilized by multiple intramolecular disulfide bonds. It has been shown that the knob domain itself can function as an independent binding peptide, even when excised or isolated from the remainder of the antibody variable domain. In provided embodiments, a knob domain (or knob domain peptide) is a peptide sequence that corresponds to, or is derived from, such an ultralong CDR3 antibody knob region and that retains an autonomous, disulfide-constrained fold capable of specific binding to a target molecule. The knob domain thus may be a shorter peptide sequence and is not limited to their natural antibody context but that has been derived from an antibody with an ultralong CDR3 sequence. In species with ultralong CDR3 antibodies, such as cow antibodies, the ultralong CDR3 sequences forms a structure where a subdomain with an unusual architecture is formed from a “stalk”, composed of two 12-residue, anti-parallel β-strands (ascending and descending strands), and a longer, e.g., 39-residue, disulfide-rich “knob” that sits atop the stalk, far from the canonical antibody paratope. The long anti-parallel β-ribbon serves as a bridge to link the knob domain with the main antibody scaffold. The unique “stalk and knob” structure of the ultralong CDR3 results in the two antiparallel β-strands (an ascending and descending stalk strand) supporting a disulfide bonded knob protruding out of the antibody surface to form a mini antigen binding domain. In some embodiments, the ultralong CDR3 antibodies comprise, in order, an ascending stalk region, a knob region, and a descending stalk region.

In some embodiments, the ultralong CDR-H3 includes an ascending stalk domain (Stalk A), a disulfide-rich knob region, and a descending stalk domain (Stalk B), in which the knob region is positioned between the ascending and descending stalk domains. In some embodiments, the sequence of the ultralong CDR-H3 provides a structure of an anti-parallel β-strands that protrude away from the antibody, in which the disulfide-rich knob region is positioned at the tip of the antibody. Stalk A comprises mainly hydrophobic side chains and a relatively conserved motif at the base, which initiates the ascending strand. This conserved motif is typically found following the first cysteine residue in variable region sequences of the various bovine or cow sequences. In some embodiments, the base of Stalk A contains residues CTTVHQ (SEQ ID NO: 38), CATVHQ (SEQ ID NO: 39), CAIVQQ (SEQ ID NO: 40), or CATVDQ (SEQ ID NO: 41) that stabilizes the base by interacting with residues of the CDR-H1. The Stalk A is connected by a variable number of residues, e.g., 2 to 8 amino acid residues, before a first conserved cysteine residue that forms part of the disulfide-bonded knob region. In some embodiments, the knob region includes a first conserved amino acid motif Cys-Pro (CP), in which the initial cysteine residue forms the first disulfide bond with another cysteine residue in the knob. The knob may include 2-12 cysteine residues that are able to form 2-6 disulfide bonds. The stalk can be of variable length, and Stalk B may comprise alternating aromatics that form a ladder through stacking interactions, that may contribute to the stability of the long solvent-exposed, two stranded β-ribbon (Wang et al. Cell. 2013, 153 (6): 1379-1393). In some embodiments, the Stalk B contains a conserved pattern of alternating tyrosines, sometimes with the motif YX₁YX₂Y (SEQ ID NO: 42), that support the knob structure.

In some embodiments, the ultralong CDR3 comprises a peptide sequence of 25-70 amino acids. In some embodiments, the ultralong CDR3 is a peptide sequence that is between or between about 35 and 70 amino acids in length, 40 and 70 amino acids in length, 45 and 70 amino acids in length, 50 and 70 amino acids in length, 55 and 70 amino acids in length, or 60 and 70 amino acids in length.

In some embodiments, the DRP is a knob peptide that is derived from the knob region of an ultralong CDR3 of a VH chain an antibody. Any of a variety of methods known to a skilled artisan can be used to obtain or identify an antibody with an ultralong CDR3. In some embodiments, an antibody with an ultralong CDR3 is obtained by immunizing a member of the Bovinae subfamily including in particular bovine species of the genus Bos, such as Bos taurus (domestic cattle, e.g., a cow), and isolating antibodies with an ultralong CDR3. In some cases, display libraries of antibody sequences, or those enriched in antibodies with ultralong CDR3, can be prepared and screened for desired bind and activity to identify or obtain an antibody with an ultralong CDR3. In some embodiments, a knob domain peptide from an ultralong CDR3 of a antibody is obtained or isolated.

Knob domain peptides can be obtained or identified from naturally occurring antibodies containing ultralong CDR3 regions, such as antibodies of a member of the subfamily Bovinae, including in particular bovine members of the genus Bos, for example Bos taurus (domestic cattle, e.g., a cow). Examples of animals that are a source of antibodies with ultralong CDR3 include any member of the Bovinae subfamily, such as cattle (Bos genus), bison (Bison), yak (Bos grunniens), water buffalo (Bubalus), gaur (Bos gaurus), and several large antelopes (like kudu, eland, and nilgai). In some embodiments, the knob domain peptide is from a naturally occurring ultralong CDR3 antibody of Bos taurus, such as domestic castle including cows, bulls, steers or heifers. In some embodiments, the knob domain peptide is obtained directly from a naturally occurring ultralong CDR3 antibody, while in other embodiments the knob domain peptide is synthetically produced, recombinantly expressed, or engineered based on, but not limited to, the sequences of naturally occurring ultralong CDR3 antibodies. Accordingly, the provided embodiments encompasses knob domain peptides in their natural, synthetic, recombinant, or engineered forms. Such peptides may be isolated directly from ultralong CDR3 immunoglobulins, recombinantly expressed based on known knob domain sequences, or designed as variants, derivatives, or synthetic mimetics thereof. A knob domain peptide as described herein may therefore be naturally occurring, recombinantly produced, comprise sequence variants that retain the characteristic disulfide-stabilized fold, or be a hybrid peptide in which natural knob sequences are combined with heterologous peptide elements. Typically, knob domain peptides are 20-70 amino acids in length and enriched in cysteine residues that form two or more intramolecular disulfide bonds, thereby constraining the domain in a stable, globular structure. This autonomous folding property distinguishes knob domain peptides from most other antibody-derived peptides, which require a full variable domain context to retain structure and function.

In some embodiments, the knob portion of such ultralong CDR3s may be identified in silico or by sequence analysis using a framework-based algorithm. In certain embodiments, the algorithm comprises identifying the conserved cysteine in framework 3 of the heavy chain variable region and the conserved tryptophan in framework 4, and then determining the sequence of the CDR3 knob domain. The knob domain is defined as an amino acid sequence of length K, beginning at position X+1 and ending at X+K, where K=L−2X. In this definition, L represents the number of amino acids in the sequence beginning with the conserved cysteine in framework 3 and ending with the conserved tryptophan in framework 4, while X represents the number of amino acids from the first cysteine in framework 3 to the first conserved cysteine encoded by the DH region in CDR H3. This approach allows the knob domain to be identified as a discrete peptide region within the ultralong CDR3 of an antibody, such as a bovine antibody.

In provided embodiments, a knob peptide is an independently produced linear disulfide-bonded peptide that is 20-70 amino acids in length, and contains 2-6 disulfide bonds formed by at least 4 non-canonical Cys residues, such as 6, 8, 10 or up to 12 non-canonical cysteine residues. Typically, knob domain peptides have a length of 20 amino acids, 25 amino acids, 30 amino acids, 35 amino acids, 40 amino acids, 45 amino acids, 50 amino acids, 55 amino acids, 60 amino acids, 65 amino acids, 70 amino acids, or any length between any of the foregoing. Typically, knob domain peptides are from 30 to 65 amino acids in length, such as 32 to 60 amino acids, for example 35 to 55 amino acids in length. In some embodiments, the knob domain is 20 to 50 amino acids in length, such as 22 to 45 amino acids or 25 to 40 amino acids. The sequences are enriched in cysteine residues, usually containing at least two, three, four, or more cysteines, which form intramolecular disulfide bonds to stabilize the globular domain. The one or more cysteine residues are distributed in a manner that produces a disulfide bonding pattern, thereby defining a “cysteine motif.” The spacing between cysteine residues may vary, for example, between one and twenty amino acids, but the overall pattern allows the formation of intramolecular disulfide linkages that constrain the peptide into a compact fold. The peptide sequence includes two to twelve cysteine residues, which are arranged so as to form one, two, three, four, five, or six intramolecular disulfide bonds. In some embodiments, a knob domain peptide comprises two to six cysteine residues arranged such that they form two, three, or more intramolecular disulfide bonds. These disulfide bonds create a structurally constrained, globular domain that is stable even when excised from its parent protein context. In certain embodiments, the cysteine residues occur in conserved positions within the domain, creating a “disulfide core” that constrains the fold. In some embodiments, one or more aromatic residues, such as tyrosine, phenylalanine, or tryptophan, are present at conserved positions to contribute to the hydrophobic core of the domain. In certain embodiments, glycine or proline residues are present at positions that promote turns or loop structures, thereby accommodating the compact, globular architecture of the knob domain. These conserved or structurally defined positions are understood in the art as locations within the knob sequence that are often observed across natural ultralong CDR3 knob domains, such as bovine ultralong CDR3 antibodies, and are necessary to maintain the disulfide-stabilized three-dimensional fold.

In some embodiments, the knob domain peptide may be extended at its N-terminus, C-terminus, or both, in order to enhance folding, stability, or binding activity. For example, the knob domain may be extended by about 1 to 30 amino acids, such as 1 to 15 amino acids, at one or both termini. Such extensions may include sequences that stabilize the autonomous fold of the knob domain, such as residues capable of interacting due to the close spatial proximity of the N- and C-termini in the folded peptide. In some embodiments, the N- and/or C-terminal extension includes a peptide sequence of about 1 to 30 amino acids, such as a short sequence of 2 to 6 amino acids, or a longer sequence of about 14 to 30 amino acids comprising a heptad repeat motif that facilitates formation of an antiparallel coiled-coil structure. For example, in some embodiments the binding peptide comprises the knob region of an ultralong CDR3 together with one, two, three, four, or five contiguous amino acids on the N-terminus and/or C-terminus. In some embodiments, the extended N- and/or C-terminal sequences are derived from a contiguous portion of the ascending stalk (stalk A) and/or descending stalk (stalk B) regions that flank the knob domain within the ultralong CDR3. For example, in some embodiments the binding peptide comprises the knob region of an ultralong CDR3 together with one, two, three, four, or five contiguous amino acids on the N-terminus and/or C-terminus derived from stalk A or stalk B. A knob domain DRP thus may include both the core disulfide-stabilized knob and knob-containing peptides with minimal or extended flanking sequences.

In some embodiments, the knob domain includes a cysteine motif. In some embodiments, the cysteine motif includes 2-20 cysteine residues, for instance between or between about 2 and 18, 2 and 16, 2 and 14, 2 and 12, 2 and 10, 2 and 8, 2 and 6, 2 and 4, 4 and 20, 4 and 18, 4 and 16, 4 and 14, 4 and 12, 4 and 10, 4 and 8, 4 and 6, 6 and 20, 6 and 18, 6 and 16, 6 and 14, 6 and 12, 6 and 10, 6 and 8, 8 and 20, 8 and 18, 8 and 16, 8 and 14, 8 and 12, 8 and 10, 10 and 20, 10 and 18, 10 and 16, 10 and 14, 10 and 12, 12 and 20, 12 and 18, 12 and 16, 12 and 14, 14 and 20, 14 and 18, 14 and 16, 16 and 20, 16 and 18, or 18 and 20 cysteine residues, each inclusive. In some embodiments, the cysteine motif includes 2-12 cysteine residues.

In some embodiments, the knob includes 2-10 disulfide bonds, for instance between or between about 2 and 9, 2 and 8, 2 and 7, 2 and 6, 2 and 5, 2 and 4, 2 and 3, 2 and 2, 2 and 10, 2 and 9, 2 and 8, 2 and 7, 2 and 6, 2 and 5, 2 and 4, 2 and 3, 3 and 10, 3 and 9, 3 and 8, 3 and 7, 3 and 6, 3 and 5, 3 and 4, 4 and 10, 4 and 9, 4 and 8, 4 and 7, 4 and 6, 4 and 5, 5 and 10, 5 and 9, 5 and 8, 5 and 7, 5 and 6, 6 and 10, 6 and 9, 6 and 8, 6 and 7, 7 and 10, 7 and 9, 7 and 8, 8 and 10, 8 and 9, or 9 and 10 disulfide bonds, each inclusive. In some embodiments, the knob domain includes 2-6 disulfide bonds.

In some embodiments, the knob domain with the cysteine motif is between the ascending and descending stalk domains. In some embodiments, the ascending stalk domain includes the sequence CX₂TVX₅Q (SEQ ID NO: 43), wherein X₂ and X₅ are any amino acid. In some embodiments, X₂ is Ser, Thr, Gly, Asn, Ala, or Pro, and X₅ is His, Gln, Arg, Lys, Gly, Thr, Tyr, Phe, Trp, Met, Ile, Val, or Leu (SEQ ID NO: 44). In some embodiments, X₂ is Ser, Ala, or Thr, and X₅ is His or Tyr (SEQ ID NO: 45).

In some embodiments, the knob domain DRPs, e.g., including those derived from ultralong CDR3, are derived from a member of the subfamily Bovinae, including in particular bovine antibodies. In some embodiments, the DRPs are produced by amplifying sequences from a complementary DNA (cDNA) library from such Bovinae subfamily member, for example a bovine, e.g. a cow complementary DNA (cDNA) library. In some embodiments, the CDR3 knob is encoded by a sequence that has been amplified from a cDNA template library prepared from RNA isolated from peripheral blood mononuclear cells (PBMCs) from an immunized animal of the subfamily Bovinae, including in particular bovine members of the genus Bos, for example Bos taurus (domestic cattle, e.g., a cow). In some embodiments, the CDR3 knob is encoded by a sequence that has been amplified from a cow cDNA template library, e.g., one prepared from RNA isolated from peripheral blood mononuclear cells (PBMCs) from an immunized cow. In some embodiments, the cDNA template library is synthesized using a pool of immunoglobulin-specific primers. In some embodiments, the cDNA template library is synthesized using a pool of IgM, IgA, and IgG-specific primers. Exemplary primers for use include those with sequences set forth in SEQ ID NO: 46 (IgG), SEQ ID NO: 47 (IgM), SEQ ID NO: 48 (IgA), and SEQ ID NO: 49 (IgG). In some embodiments, the CDR3 knob includes all or a portion of sequences that have been amplified from a cDNA template library according to any of the methods provided herein. In some embodiments, the CDR3 knob includes all or a portion of sequences that have been amplified from a cow cDNA template library according to any of the methods provided herein.

In some embodiments, the amplifying is performed by amplifying sequences encoding CDR3 knobs. In some embodiments, primers specific for the ascending and descending stalk domains of a CDR3 region, such as a cow CDR3 region, are used to amplify the sequences encoding CDR3 knobs. In some embodiments, the CDR3 knob comprises a portion of the ascending stalk domain, such as 1, 2, 3, 4, 5 or 6 amino acids. In some embodiments, the CDR3 knob comprises a portion of the descending stalk domain, such as 1, 2, 3, 4, 5, 6, 7, 8, or 9 amino acids. In some embodiments, the ascending stalk domain includes the sequence CX₂TVX₅Q, wherein X₂ and X₅ are any amino acid. In some embodiments, X₂ is Ser, Thr, Gly, Asn, Ala, or Pro, and X₅ is His, Gln, Arg, Lys, Gly, Thr, Tyr, Phe, Trp, Met, Ile, Val, or Leu. In some embodiments, X₂ is Ser, Ala, or Thr, and X₅ is His or Tyr. In some embodiments, the primers used for amplifying include or consist of the sequences set forth in SEQ ID NO: 50-54. In some embodiments, the primers used for amplifying include or consist of the sequences set forth in SEQ ID NO: 51-54. In some embodiments, the primers used for amplifying include or consist of the sequences set forth in SEQ ID NO: 55-64. In some embodiments, the primers used for amplifying include or consist of the sequences set forth in SEQ ID NO: 57, 61, and 62.

In some embodiments, the primers used for amplifying are a pool of different primers specific for the ascending and descending stalk domains. In some embodiments, the pool of primers contains at least two, three, four, five, six, seven, eight, nine, or 10 different primers. In some embodiments, the pool of primers contains at least two, three, four, five, six, seven, eight, nine, or 10 different primers from the primers set forth in SEQ ID NO: 50-64. In some embodiments, the pool of primers contains at least two, three, four, five, six, or seven different primers from the primers set forth in SEQ ID NO: 51-54, 57, 61, and 62. In some embodiments, the pool of primers contains the primers set forth in SEQ ID NO: 51-54. In some embodiments, the pool of primers contains the primers set forth in SEQ ID NO: 57, 61, and 62. In some embodiments, the pool of primers contains the primers set forth in SEQ ID NO: 51-54, 57, 61, and 62.

In some embodiments, the provided methods include the use of or amplification from a cDNA template library that is prepared from RNA isolated from an immunized member of the subfamily Bovinae, including in particular bovine species of the genus Bos such as Bos taurus (domestic cattle, e.g., cows, bulls, steers, or heifers). In some embodiments, the provided methods include the use of or amplification from a cDNA template library that is prepared from RNA isolated from an immunized cow. In some embodiments, the methods further include immunizing the animal, such as a cow, with a target antigen.

In some embodiments, the animal, such as a cow, is immunized with a target antigen. In some embodiments, the target antigen is a nonvirulent bacteria, a virus, a viral protein, a cancer antigen, a human IgG, or a recombinant protein thereof. In some embodiments, the target antigen is a virus or viral protein, e.g., that is associated with a coronavirus, e.g., SARS CoV-2. In some embodiments, the cow is immunized with multiple target antigens, for instance different viral antigens. In some embodiments, the different viral antigens are proteins associated with different variants, clades, or strains of a virus.

In some embodiments, the target antigen is a coronavirus, a coronavirus pseudovirus, or an antigen of such virus, such as a recombinant coronavirus Spike protein, or a receptor-binding domain (RBD) of a coronavirus Spike protein. Coronaviruses may be from the subfamily Orthocoronavirinae, which is one of two sub-families in the family Coronaviridae, order Nidovirales, and realm Riboviria. There are four genera: Alphacoronavirus, Betacoronavirus, Gammacoronavirus and Deltacoronavirus. SARS CoV2 is a Betacoronavirus, belonging to the subgenus Sarbecovirus. In some embodiments, the coronavirus is selected from the group consisting of 229E, NL63, OC43, HKU1, MERS-CoV, SARS-CoV, and SARS-CoV2. In some embodiments, the coronavirus is a SARS-CoV2 selected from Wuhan-Hu-2 isolate, B.1.352 South African variant, or B.1.1.7 UK variant. In some embodiments, the SARS CoV-2 specific antigen comprises a S trimer polypeptide. In some embodiments, the SARS CoV-2 specific antigen comprises a S monomer polypeptide. In some embodiments, the SARS CoV-2 specific antigen comprises a polynucleotide encoding a S trimer or monomer polypeptide. In some embodiments, the cow is immunized with multiple target antigens associated with any combination of coronaviruses 229E, NL63, OC43, HKU1, MERS-CoV, SARS-CoV, and SARS-CoV2. In some embodiments, the cow is immunized with multiple target antigens associated with any combination of SARS-CoV2 variants selected from Wuhan-Hu-2 isolate, B.1.352 South African variant or B.1.1.7 UK variant.

In some embodiments, the antigen is a cancer antigen. In some embodiments, the antigen is selected from among ACTHR, endothelial cell Anxa-1, aminopeptidase N, anti-IL-6R, alpha-4-integrin, alpha-5-beta-3 integrin, alpha-5-beta-5 integrin, alpha-fetoprotein (AFP), ANPA, ANPB, APA, APN, APP, 1AR, 2AR, AT1, B1, B2, BAGE1, BAGE2, B-cell receptor BB1, BB2, BB4, calcitonin receptor, cancer antigen 125 (CA 125), CCK1, CCK2, CD5, CD10, CD11a, CD13, CD14, CD19, CD20, CD22, CD25, CD30, CD33, CD38, CD45, CD52, CD56, CD68, CD90, CD133, CD7, CD15, CD34, CD44, CD206, CD271, CEA (CarcinoEmbryonic Antigen), CGRP, chemokine receptors, cell-surface annexin-1, cell-surface plectin-1, Cripto-1, CRLR, CXCR2, CXCR4, DCC, DLL3, E2 glycoprotein, EGFR, EGFRvIII, EMR1, Endosialin, EP2, EP4, EpCAM, EphA2, ET receptors, Fibronectin, Fibronectin ED-B, FGFR, frizzled receptors, GAGE1, GAGE2, GAGE3, GAGE4, GAGE5, GAGE6, GLP-2 receptor, G-protein coupled receptors of the Family A (Rhodopsin-like), G-protein coupled receptors of the Family B (Secretin receptor-like) like), G-protein coupled receptors of the Family C (Metabotropic Glutamate Receptor-like), GD2, GP100, GP120, Glypican-3, hemagglutinin, Heparin sulfates, HER1, HER2, HER3, HER4, HMFG, HPV 16/18 and E6/E7 antigens, hTERT, IL11-R, IL-13R, ITGAM, Kalikrien-9, Lewis Y, LH receptor, LHRH-R, LPA1, MAC-1, MAGE 1, MAGE 2, MAGE 3, MAGE 4, MART1, MC1R, Mesothelin, MUC1, MUC16, Neu (cell-surface Nucleolin), Neprilysin, Neuropilin-1, Neuropilin-2, NG2, NK1, NK2, NK3, NMB-R, Notch-1, NY-ESO-1, OT-R, mutant p53, p97 melanoma antigen, NTR2, NTR3, p32 (p32/gC1q-R/HABP1), p75, PAC1, PAR1, Patched (PTCH), PDGFR, PDFG receptors, PDT, Protease-cleaved collagen IV, proteinase 3, prohibitin, protein tyrosine kinase 7, PSA, PSMA, purinergic P2X family (e.g., P2X1-5), mutant Ras, RAMP1, RAMP2, RAMP3 patched, RET receptor, plexins, smoothened, sst1, sst2A, sst2B, sst3, sst4, sst5, substance P, TEMs, T-cell CD3 Receptor, TAG72, TGFBR1, TGFBR2, Tie-1, Tie-2, Trk-A, Trk-B, Trk-C, TR1, TRPA, TRPC, TRPV, TRPM, TRPML, TRPP (e.g., TRPV1-6, TRPA1, TRPC1-7, TRPM1-8, TRPP1-5, TRPML1-3), TSH receptor, VEGF receptors (VEGFR2 or Flt-1, VEGFR2 or FLK-1/KDR, and VEGF-3 or FLT-4), voltage-gated ion channels, VPAC1, VPAC2, Wilms tumor 1, Y1, Y2, Y4, and Y5.

In some embodiments, the antigen is HER1/EGFR, HER2/ERBB2, CD20, CD25 (IL-2Rα receptor), CD33, CD52, CD133, CD206, CEA, CEACAM1, CEACAM3, CEACAM5, CEACAM6, cancer antigen 125 (CA125), alpha-fetoprotein (AFP), Lewis Y, TAG72, Caprin-1, mesothelin, PDGF receptor, PD-1, PD-L1, CTLA-4, IL-2 receptor, vascular endothelial growth factor (VEGF), CD30, EpCAM, EphA2, Glypican-3, gpA33, mucins, CAIX, PSMA, folate-binding protein, gangliosides (such as GD2, GD3, GM2 and GM2), VEGF receptor (VEGFR), integrin αVβ3, integrin α5β1, ERBB3, MET, IGF1R, EPHA3, TRAILR1, TRAILR2, RANKL, FAP, tenascin, AFP, BCR complex, CD3, CD18, CD44, CTLA-4, gp72, HLA-DR 10 β, HLA-DR antigen, IgE, MUC-1, nuC242, PEM antigen, metalloproteinases, Ephrin receptor, Ephrin ligands, HGF receptor, CXCR4, CXCR4, Bombesin receptor, and SK-2 antigen.

In some embodiments, the antigen is CD25, PD-2 (CD279), PD-L2 (CD274, B7-H1), PD-L2 (CD273, B7-DC), CTLA-4, LAG3 (CD223), TIM3 (HAVCR2), 4-1BB (CD137, TNFRSF9), CXCR2, CXCR4 (CD184), CD27, CEACAM1, Galectin 9, BTLA, CD160, VISTA (PD2 homologue), B7-H4 (VCTN1), CD80 (B7-1), CD86 (B7-2), CD28, HHLA2 (B7-H7), CD28H, CD155, CD226, TIGIT, CD96, Galectin 3, CD40, CD40L, CD70, LIGHT (TNFSF14), HVEM (TNFRSF14), B7-H3 (CD276), Ox40L (TNFSF4), CD137L (TNFSF9, GITRL), B7RP1, ICOS (CD278), ICOSL, KIR, GAL9, NKG2A (CD94), GARP, TL1A, TNFRSF25, TMIGD2, BTNL2, Butyrophilin family, CD48, CD244, Siglec family, CD30, CSF1R, MICA (MHC class I polypeptide-related sequence A), MICB (MHC class I polypeptide-related sequence B), NKG2D, KIR family (Killer-cell immunoglobulin-like receptor, LILR family (Leukocyte immunoglobulin-like receptors, CD85, ILTs, LIRs), SIRPA (Signal regulatory protein alpha), CD47 (IAP), Neuropilin 2 (NRP-1), a VEGFR, and VEGF.

In some embodiments, the antigen is an immunomodulatory protein (e.g. a checkpoint molecule). In some embodiments, the antigen is an immune checkpoint receptor ligand. Illustrative immune checkpoint molecules that may be targeted for blocking or inhibition include, but are not limited to, PD2 (CD279), PDL2 (CD274, B7-H1), PDL2 (CD273, B7-DC), CTLA-4, LAG3 (CD223), TIM3, 4-1BB (CD137), 4-1BBL (CD137L), GITR (TNFRSF18, AITR), CD40, Ox40 (CD134, TNFRSF4), CXCR2, tumor associated antigens (TAA), B7-H3, B7-H4, BTLA, HVEM, GAL9, B7H3, B7H4, VISTA, KIR, 2B4 (belongs to the CD2 family of molecules and is expressed on all NK, γδ, and memory CD8+ (αβ) T cells), CD160 (also referred to as BY55) and CGEN-15049. In some embodiments, the immune checkpoint molecule is CD25, PD-1, PD-L1, PD-L2, CTLA-4, LAG-3, TIM-3, 4-1BB, GITR, CD40, CD40L, OX40, OX40L, CXCR2, B7-H3, B7-H4, BTLA, HVEM, CD28 and VISTA.

Once identified, the knob peptide sequences can be amplified using methods known to a skilled artisan. In other embodiments, the knob peptide may be synthetically generated. A variety of techniques including recombinant methods, chemical synthesis, or combinations thereof, may be employed. In some embodiments, chemical synthesis methods may include known chemical synthesis techniques, such as the phosphonamidite method. In some instances, a recombinant or synthetic nucleic acid may be generated through polymerase chain reaction (PCR).

In some embodiments, an animal, such as bovine animals (e.g. cow) is immunized by administering at least one dose of an antigenic composition comprising a target antigen or a group of related target antigens, e.g., antigens associated with variants of a virus. In some embodiments, the antigenic composition further comprises an adjuvant. The skilled person is familiar with many potentially useful adjuvants, such as Freund's complete adjuvant, alum, and squalene. Sec, e.g., US Patent Appl. Pub. No. 20150361160, which is incorporated by reference herein in its entirety for all purposes. Adjuvants which may be used in compositions of the invention include but are not limited to oil emulsion compositions (oil-in-water emulsions and water-in-oil emulsions), complete Freund's adjuvant (CFA) and incomplete Freund's adjuvant (IFA). In one embodiment, the adjuvant comprises RIBI, Iscomatrix, or ENABL CI (VaxLiant). Adjuvants suitable for use in the invention include bacterial or microbial derivatives such as derivatives of enterobacterial lipopolysaccharide (LPS), Lipid A derivatives, immunostimulatory oligonucleotides and ADP-ribosylating toxins and detoxified derivatives thereof.

In some embodiments, the animal is a member of the subfamily Bovinae, including but not limited to domestic cattle (Bos taurus), bison (Bison bison), African buffalo (Syncerus caffer), water buffalo (Bubalus bubalis), or yak (Bos grunniens). In certain embodiments, the bovine is domestic cattle. In some embodiments, the domestic cattle is a dairy animal, such as a dairy cow. In some embodiments, the animal is a pregnant cow.

Methods for immunizing such animals, for example a bovine, such as a cattle, to produce, for example, high titer colostrum, milk, serum, or immune tissues (e.g., PBMC), are known in in the art. Such methods are disclosed, for example, in US Patent Appl. Pub. Nos US20070053917 and US20130022619, each of which is incorporated by reference herein in its entirety for all purposes.

In some embodiments, the immunizing comprises administering a priming dose and at least one booster dose of the antigenic composition. In some embodiments, the immunizing comprises administering more than one booster doses of the antigenic composition. In one embodiment, the priming dose and at least one booster dose comprise the same antigenic composition. In some embodiments, the more than one booster doses comprise the same antigenic composition. The animal may be dosed with the immunogenic composition at intervals over a period of days, weeks, or months. At the conclusion of the immunization regime, the hyperimmune material such as blood, milk or colostrum is harvested. In one embodiment, the hyperimmune material is collected less than 2 months, less than 3 months, less than 4 months, less than 5 months, less than 6 months, less than 9 months, or less than 12 months after administering the priming dose. In one embodiment, the hyperimmune material is collected between about 3 months and about 6 months after administering the priming dose. In one embodiment, the hyperimmune material is collected between about 3 months and about 9 months after administering the priming dose. In some embodiments, the hyperimmune material is collected between about 3 months and about 12 months after administering the priming dose. In one embodiment, the hyperimmune material is collected between about 6 months and about 12 months after administering the priming dose.

In some embodiments, the methods further comprise isolating from the animal, such as a bovine (e.g., cow) a biological sample. In some embodiments, the biological sample is milk, blood, serum, colostrum, or peripheral blood mononuclear cells (PBMC). In one embodiment, the biological sample is collected less than 2 months, less than 3 months, less than 4 months, less than 5 months, less than 6 months, less than 9 months, or less than 12 months after administering the priming dose. In one embodiment, the biological sample is collected between about 3 months and about 6 months after administering the priming dose. In some embodiments, the biological sample is collected between about 3 months and about 9 months after administering the priming dose. In some embodiments, the biological sample is collected between about 3 months and about 12 months after administering the priming dose. In some embodiments, the biological sample is collected between about 6 months and about 12 months after administering the priming dose.

In some embodiments, the methods further include isolating a peripheral blood mononuclear cell (PBMC) from the animal, such as a bovine (e.g., cow), and cloning a polynucleotide that encodes an antibody, e.g., containing an ultralong CDR3 with a knob sequence. In embodiments, RNA may be isolated from the PBMCs obtained from the immunized animal and subjected to PCR amplification to enrich for ultralong CDRs or knob domain peptide sequences as described above. In one embodiment, the cloning the polynucleotide comprises performing single-cell RT-PCR amplification.

b. Synthetic DRPs

In some embodiments, the disulfide rich protein as part of the CIS Display library produced in the provided methods is a soluble synthetic or semisynthetic peptide. In some embodiments, the disulfide rich protein is a semisynthetic or modified CDR3 knob. In some embodiments, the disulfide rich protein is a semisynthetic CDR3 knob. In some embodiments, the semisynthetic CDR3 knob is derived from a bovine CDR3 knob that has been used as a scaffold for modifications.

In some embodiments, the bovine CDR3 knob is encoded by a sequence that has been amplified from a bovine (e.g., cow) cDNA template library, e.g., one prepared from RNA isolated from peripheral blood mononuclear cells (PBMCs) from an immunized bovine, such as a cow. In some embodiments, the bovine CDR3 knob includes all or a portion of sequences that have been amplified from the cDNA template library according to any of the methods provided herein. In some embodiments, the bovine CDR3 knob is any that has been identified or selected as a binder of a target molecule. In some embodiments, the bovine CDR3 knob is or is a portion of any CDR3 knob that has been identified or selected as a binder of a target molecule according to any of the methods provided herein.

In some embodiments, the bovine CDR3 knob has been modified to include random mutations, e.g., while preserving the cysteine motif and disulfide bond structure as described herein, e.g., such that the semisynthetic ultralong CDR3 knob still includes 4-20 cysteine residues and 2-10 disulfide bonds. In some embodiments, the bovine CDR3 knob has been modified to include an exogenous peptide sequence. In some embodiments, the bovine CDR3 knob has been modified to delete one or more peptide sequences therein, e.g., while preserving the cysteine motif and disulfide bond structure as described herein, e.g., such that the semisynthetic CDR3 knob still includes 4-20 cysteine residues and 2-10 disulfide bonds.

In some embodiments, the synthetic peptide is a random sequence polypeptide with a cysteine motif and disulfide bonds as described herein, e.g., with 2-20 cysteine residues and 2-10 disulfide bonds. In some embodiments, the synthetic peptide has been selected from a random sequence library for having a cysteine motif and disulfide bonds, e.g., for having 2-20 cysteine residues and 2-10 disulfide bonds. Methods of producing DNA constructs encoding a random sequence are known.

c. Cyclotides DRPs

In some embodiments, the disulfide rich protein screened and produced in the provided methods is a cyclotide. In some embodiments, the cyclotide is a cyclotide that has been modified to include an exogenous peptide sequence.

Cysteine-knot microproteins (cyclotides) include a naturally occurring family of cysteine-knot microproteins or cyclotides found in various plant species. Cysteine-knot microproteins (cyclotides) are small peptides, typically consisting of about 30-40 amino acids, which can be found naturally as cyclic or linear forms, where the cyclic form has no free N- or C-terminal amino or carboxyl end. They have a defined structure based on three intra-molecular disulfide bonds and a small triple stranded β-sheet (Craik et al., 2001; Toxicon 39, 43-60). The cyclic proteins exhibit conserved cysteine residues defining a structure referred to herein as a “cysteine knot”. This family includes both naturally occurring cyclic molecules and their linear derivatives as well as linear molecules which have undergone cyclization. These molecules are useful as molecular framework structures having enhanced stability over less structured peptides (Colgrave and Craik, 2004; Biochemistry 43, 5965-5975).

The main cyclotide features are a remarkable stability due to the cysteine knot, a small size making them readily accessible to chemical synthesis, and an excellent tolerance to sequence variations. The cyclotide scaffold is found in almost thirty different protein families among which conotoxins, spider toxins, squash inhibitors, agouti-related proteins and plant cyclotides are the most populated families. Cyclotides from plants in the Rubiaceae and Violaceae families are for the most part found to be head-to-tail cyclic peptides (Craik et al. 2010. Cell. Mol. Life Sci. 67:9-16). However, within the squash inhibitor family of cyclotides both cyclic and linear cyclotides have been identified from Momordica cochinchinensis: the cyclic trypsin inhibitors (McoTI)-I and -II and their linear counterpart McoTI-III (Hernandez et al. 2000. Biochemistry, 39, 5722-5730). It is now clear that both cyclic and linear variants can exist in different cyclotide families, but the impact of the cyclization is poorly understood. Cyclic peptides were expected to display improved stability, better resistance to proteases, and reduced flexibility when compared to their linear counterparts, hopefully resulting in enhanced biological activities. However, linear cyclotides have the advantage of being able to be more easily linked to other peptides or proteins.

For instance, cyclotides are commonly found in plants. In aspects of provided embodiments, cyclotides are derived from linear or cyclic form of cyclotides of the Momordicae, Rubiaceae and Violaceae, plant species. In a preferred aspect, cyclotides of the invention are derived from linear or cyclic form of cyclotides of the Momordicae species including the squash serine protease inhibitor family (Otlewski & Korowarsch Acta Biochim Pol. 1996; 43 (3): 431-44), and in a more preferred aspect from Momordica cochinchinensis trypsin inhibitors McoTI-I [SEQ ID NO: 65] and -II [SEQ ID NO: 66] (naturally cyclic) and McoTI-III (naturally linear) [SEQ ID NO: 67] below.

Mcoti-I
[SEQ ID NO: 65]
GGVCPKILQRCRRDSDSPGACICRGNGYCGSGSD
Mcoti-II
[SEQ ID NO: 66]
GGVCPKILKKCRRDSDSPGACICRGNGYCGSGSD
Mcoti-III
[SEQ ID NO: 67]
ERACPRILKKCRRDSDSPGACICRGNGYCG

In some embodiments, the cyclotide molecular framework comprising a sequence of amino acids or analogues thereof forming a cysteine-knot backbone wherein said cysteine-knot backbone comprises sufficient disulfide bonds or chemical equivalents thereof, to confer a knotted topology on the three-dimensional structure of said cysteine-knot backbone and wherein at least one exposed amino acid residue such as on one or more beta turns and/or within one or more loops, is inserted or substituted (replaced) relative to the naturally occurring amino acid sequence. In some embodiments, the cyclotide is modified by the insertion of or substitution with an exogenous peptide sequence. Hence, the cyclotides described herein are modified cyclotides compared to a natural or wildtype unmodified cyclotide, in which the modified cyclotide has one or more loops inserted or substituted by one or more amino acid sequences, e.g., an exogenous peptide sequence. In aspects of provided embodiments, the modified cyclotides incorporate sufficient amino acid structure to provide high enzymatic stability.

In some embodiments, the modified cyclotide sequence may be defined as having a cysteine knot backbone moiety and an exogenous peptide sequence, said modified cyclotide comprising: i) an exogenous peptide sequence, wherein said sequence is about 2 to 50 amino acid residues; and ii) a cysteine knot backbone grafted to said sequence of step i), wherein said cysteine knot backbone comprises the structure (I):

(X^{V}₁...X^{V}_{f})C₁(X₁...Xₐ)C₂(X^{I}₁...X^{I}_{b})C₃(X^{II}₁...X^{II}_{c})C₄(X^{III}₁...X^{III}_{d})C₅(X^{IV}₁...X^{IV}ₑ)C₆(X^{V}₁...X^{V}_{f})
Loop6 Loop1 Loop2 Loop3 Loop4 Loop5 Loop6

wherein C₁ to C₆ are cysteine residues; wherein each of C₁ and C₄, C₂ and C₅, and C₃ and C₆ are connected by a disulfide bond to form a cysteine knot; wherein each X represents an amino acid residue in a loop, wherein said amino acid residues are the same or different; wherein d is about 1-2; wherein one or more of loops 1, 2, 3, 5 or 6 have an amino acid sequence comprising the sequence of clause i), wherein any loop comprising said sequence of clause i) comprises 2 to about 50 amino acids, and wherein for any of loops 1, 2, 3, 5, or 6 that do not contain said sequence of clause i), a, b, c, e, and f, are the same or different, and are each any number from 3-10, and b, c, e, and f are each any number from 1 to 20.

In some embodiments, the modified cyclotide sequence may be either linear or cyclic.

In some embodiments, modified cyclotides are derived from linear or cyclic forms of cyclotides of the Momordicae, Rubiaceae, and Violaceae plant species. In some embodiments, the modified cyclotides are derived from linear or cyclic form of cyclotides of the Momordicae species, including the squash serine protease inhibitor family (Otlewski & Korowarsch Acta Biochim Pol. 1996; 43 (3): 431-44). In some embodiments, the modified cyclotides are derived from Momordica cochinchinensis trypsin inhibitors McoTI-I [SEQ ID NO: 65] and -II [SEQ ID NO: 66] (naturally cyclic) and McoTI-III (naturally linear) [SEQ ID NO: 67] below.

Mcoti-I
[SEQ ID NO: 65]
GGVCPKILQRCRRDSDSPGACICRGNGYCGSGSD
Mcoti-II
[SEQ ID NO: 66]
GGVCPKILKKCRRDSDSPGACICRGNGYCGSGSD
Mcoti-III
[SEQ ID NO: 67]
ERACPRILKKCRRDSDSPGACICRGNGYCG

For instance, the unmodified or wildtype cyclotide can be a cyclotide set forth in any one of SEQ ID NOS: 65-67 to which one or more loops thereof is inserted or substituted by one or more amino acid sequences (e.g., an exogenous peptide sequence). In particular embodiments, the modified cyclotides are derived from loop replacement libraries based on Mcoti-II (SEQ ID NO: 66).

In some embodiments, the loop into which the exogenous peptide sequence is inserted or substituted is loop 1. In some embodiments, the loop into which the exogenous peptide sequence is inserted or substituted is loop 5. In some embodiments, the loop into which the exogenous peptide sequence is inserted or substituted is loop 6, such as formed subject to cyclization.

In some embodiments, the exogenous peptide sequence that is inserted or replaced into an unmodified cyclotide, e.g. the cyclotide Mcoti-II (SEQ ID NO: 66), is 2 to 50 amino acid residues. In some embodiments, the exogenous peptide sequence is 2 to 40 amino acids, 2 to 30 amino acids, 2 to 25 amino acids, 2 to 20 amino acids, 2 to 15 amino acids, 2 to 10 amino acids, 2 to 5 amino acids, 5 to 50 amino acids, 5 to 40 amino acids, 5 to 30 amino acids, 5 to 25 amino acids, 5 to 20 amino acids, 5 to 15 amino acids, 5 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 amino acids, 25 to 30 amino acids, 30 to 50 amino acids, 30 to 40 amino acids, or 40 to 50 amino acids. In some embodiments, the exogenous peptide sequence is 2 to 30 amino acids, such as 2 to 24 amino acids, 2 to 18 amino acids, 2 to 12 amino acids, 2 to 6 amino acids, 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids.

d. scFv DRPs

In some embodiments, the disulfide rich protein as part of the CIS display library in the provided methods is a single-chain variable fragment (scFv).

In some embodiments, the scFv comprises a human scFv. In some embodiments, the human scFv is encoded by a sequence that has been amplified from human samples. In some embodiments, samples comprise a blood sample, a bone marrow sample, and/or a cord blood sample. In some embodiments, the scFv sequence is prepared from RNA isolated from samples, cDNA is synthesized from the isolated RNA, and the amplifying is done by amplifying sequences encoding scFv regions of interest.

In some embodiments, the scFv comprises a mouse scFv. In some embodiments, the mouse scFv is encoded by a sequence that has been amplified from a mouse sample or mice samples. In some embodiments, samples comprise a blood sample, a bone marrow sample, and/or a cord blood sample. In some embodiments, the scFv sequence is prepared from RNA isolated from samples, cDNA is synthesized from the isolated RNA, and the amplifying is done by amplifying sequences encoding scFv regions of interest.

In some embodiments, the scFv comprises a rat scFv. In some embodiments, the rat scFv is encoded by a sequence that has been amplified from a rat sample or samples. In some embodiments, samples comprise a blood sample, a bone marrow sample, and/or a cord blood sample. In some embodiments, the scFv sequence is prepared from RNA isolated from samples, cDNA is synthesized from the isolated RNA, and the amplifying is done by amplifying sequences encoding scFv regions of interest.

In some embodiments, the scFv comprises a rabbit scFv. In some embodiments, the rabbit scFv is encoded by a sequence that has been amplified from a rabbit sample or samples. In some embodiments, samples comprise a blood sample, a bone marrow sample, and/or a cord blood sample. In some embodiments, the scFv sequence is prepared from RNA isolated from samples, cDNA is synthesized from the isolated RNA, and the amplifying is done by amplifying sequences encoding scFv regions of interest.

In some embodiments, the scFv comprises a cow ultralong scFv. In some embodiments, the scFv includes a VH region having a cow ultralong CDR3. In some embodiments, the VH region is encoded by a sequence that has been amplified from a cow cDNA template library, e.g., one prepared from RNA isolated from peripheral blood mononuclear cells (PBMCs) from an immunized cow. In some embodiments, the amplifying is done by amplifying sequences encoding VH regions of bovine antibody families known or suspected to contain ultralong CDR3s. In some embodiments, sequences of VH regions of the IgHV1-7 family are amplified to produce sequences encoding the VH region of the scFv. In some embodiments, the VH regions of the IgHV1-7 family are amplified with a forward primer that includes the sequence set forth in SEQ ID NO: 68 and a reverse primer that includes the sequence set forth in SEQ ID NO: 69. In some embodiments, the forward primer and/or the reverse primer further include sequences specific to restriction enzyme sites in order to facilitate cloning. In some embodiments, the VH regions of the IgHV1-7 family are amplified with a forward primer set forth in SEQ ID NO: 70 and a reverse primer set forth in SEQ ID NO: 71.

In some embodiments, preparation of sequences for the VH regions of the disulfide rich protein also includes a size separation step. In some embodiments, following amplification of VH region sequences, e.g., of the IgHV1-7 family, such as from a cow cDNA template library, sequences encoding VH regions with an ultralong CDR3 are separated from shorter sequences encoding VH regions without an ultralong CDR3. In some embodiments, the size separation step further enriches for amplified sequences encoding VH regions with an ultralong CDR3.

In some embodiments, the size separation step involves separating, from sequences encoding a plurality of amplified VH regions, sequences of, of about, or greater than 425, 450, 475, 500, 525, or 550 base pairs in length, wherein the sequences of, of about, or greater than 425, 450, 475, 500, 525, or 550 base pairs in length include the sequences encoding VH regions with an ultralong CDR3. In some embodiments, sequences of, of about, or greater than 550 base pairs in length are separated from the remaining sequences.

In some embodiments, the size separation is performed by agarose gel electrophoresis. In some embodiments, a 1.2%, 1.5%, or 2% agarose gel is used. In some embodiments, a 2% agarose gel is used.

In some embodiments, the scFv includes a VL region that is fixed across the disulfide rich protein display library. In some aspects, the use of a fixed VL region improves selection and/or screening for scFvs including a VH region with an ultralong CDR3. In some embodiments, the VL region is a variable lambda light (VL) region selected from the group consisting of BLV1H12, BLV5D3, BLV8C11, BF1H1, BLV5B8, and F18, or is a humanized variant thereof. In some embodiments, the VL region is the BLV5B8 lambda VL region (SEQ ID NO: 72) or a humanized variant thereof. In some embodiments, the VL region is the BLV1H12 lambda VL region or a humanized variant thereof. In some embodiments, the BLV1H12 VL region is set forth in SEQ ID NO: 73. In some embodiments, the humanized variant comprises one or more of amino acid replacements S2A, T5N, P8S, A12G, A13S, and P14L based on Kabat numbering, amino acid replacements 129V and N32G in the CDR1 region, and/or amino acid substitution of DNN to GDT in the CDR2 region. In some embodiments, the humanized variant of BLV1H12 comprises the sequence set forth in SEQ ID NO: 74.

In some embodiments, at least or at least about 20%, 30%, 35%, 40%, 45%, 50%, 60%, 70%, 80%, 85%, 90%, or 95% of the displayed scFvs include a VH region comprising an ultralong CDR3 region. In some embodiments, at least or at least about 30% of the displayed scFvs include a VH region comprising an ultralong CDR3 region. In some embodiments, at least or at least about 40% of the displayed scFvs include a VH region comprising an ultralong CDR3 region. In some embodiments, at least or at least about 50% of the displayed scFvs include a VH region comprising an ultralong CDR3 region. In some embodiments, at least or at least about 60% of the displayed scFvs include a VH region comprising an ultralong CDR3 region. In some embodiments, at least or at least about 70% of the displayed scFvs include a VH region comprising an ultralong CDR3 region. In some embodiments, at least or at least about 80% of the displayed scFvs include a VH region comprising an ultralong CDR3 region. In some embodiments, at least or at least about 90% of the displayed scFvs include a VH region comprising an ultralong CDR3 region. In some embodiments, at least or at least about 95% of the displayed scFvs include a VH region comprising an ultralong CDR3 region.

In some embodiments, the VH and VL regions of the scFv are joined directly. In some embodiments, the VH and VL regions of the scFv are joined indirectly, e.g., via a peptide linker. In some embodiments, the peptide linker is a flexible linker. In some embodiments, the peptide linker is (Gly4 Ser)3 (SEQ ID NO: 75).

### 2. Molecular Chaperones

Molecular chaperones, or chaperones, are highly conserved molecular machines that control cellular protein homeostasis (proteostasis). Across species, they promote de novo protein folding and protein maturation, protein translocation, protein-complexes assembly and disassembly, protein disaggregation and refolding, and protein degradation.

Chaperones have been grouped into families based on their molecular mass, common domains, protein structure similarity, and common function. Families composing the main chaperone machinery, which modulate protein structure without participating in the final protein complex, include prefoldin, the small heat shock proteins (sHSP), and the main ATP-hydrolyzing chaperones, HSP60, HSP70, HSP90, and HSP100. Families of co-chaperones modulate the activity of main chaperones by regulating their ATPase cycle or the recognition, binding, or release of chaperone substrates, and include HSP10, HSP40 (DNAJ), nuclear exchange factors (NEFs), and co-HSP90. Folding enzymes that catalyze folding-accelerating reactions, such as peptidyl prolyl cis-trans isomerization or protein disulfide isomerization are also considered as chaperones.

One or more molecular chaperones can be included in the CIS display library to stabilize DRPs. In some embodiments, the one molecular chaperone is provided by expression from a DNA sequence encoding the molecular chaperone included in the plurality of DNA constructs. In other embodiments, the molecular chaperone is provided by addition of one or more DNA sequences encoding the molecular chaperone or the molecular chaperone itself to the cell-free transcription/translation environment.

In some embodiments, the one or more molecular chaperone sequences, either included in the DNA constructs or provided to cell-free transcription/translation environment, can encode molecular chaperones comprising TOR2A, TOR3A, TOR1A, TOR1B, TOR4A, LONP1, AFG3L2, ATAD3A, ATAD3B, ATAD3C, AHSA1, CDC37, CDC37L1, PTGES3, AIP, AIPL1, FKBP4, FKBP5, FKBPL, PPID, PPP5C, RPAP3, SGTA, SGTB, ST13, STIP1, STUB1, SUGT1, TOMM34, TTC1, TTC36, TTC4, TTC5, UNC45A, UNC45B, NKTR, HSPE1, DNAJA1, DNAJA2, DNAJA3, DNAJA4, DNAJB1, DNAJB11, DNAJB12, DNAJB13, DNAJB14, DNAJB2, DNAJB4, DNAJB5, DNAJB6, DNAJB7, DNAJB8, DNAJB9, DNAJC1, DNAJC10, DNAJC11, DNAJC12, DNAJC13, DNAJC14, DNAJC15, DNAJC16, DNAJC17, DNAJC18, DNAJC19, DNAJC2, DNAJC21, DNAJC22, DNAJC24, DNAJC25, DNAJC27, DNAJC28, DNAJC3, DNAJC30, DNAJC4, DNAJC5, DNAJC5B, DNAJC5G, DNAJC6, DNAJC7, DNAJC8, DNAJC9, GAK, HSCB, SACS, SEC63, BBS10, CCT2, CCT3, CCT4, CCT5, CCT6A, CCT6B, CCT7, CCT8, CCT8L2, HSPD1, MKKS, TCP1, HSPA12A, HSPA12B, HSPA13, HSPA14, HSPA1A, HSPA1B, HSPA1L, HSPA2, HSPA5, HSPA6, HSPA8, HSPA9, HSP90AA1, NAA15, NAA16, HSP90AB1, HSP90B1, TRAP1, BAG1, BAG2, BAG3, BAG4, BAG5, GRPEL1, GRPEL2, HSPA4, HSPA4L, HSPBP1, HSPH1, HYOU1, SIL1, FKBP10, FKBP11, FKBP14, FKBP15, FKBP1A, FKBP1B, FKBP2, FKBP3, FKBP6, FKBP7, FKBP8, FKBP9, FKBP1C, CALR, CANX, CLGN, EDEM1, EDEM2, EDEM3, ERP44, MZB1, P4HB, PDIA2, PDIA3, PDIA4, PDIA5, PDIA6, TXNDC5, TXNDC12, TXNRD1, TXNDC11, TMX1, TMX2, TMX3, SERPINH1, VCP, PHB, PHB2, CS1L, CLPB, CLPX, PPIA, PPIB, PPIC, PPIE, PPIF, PPIG, PPIH, PPIL1, PPIL2, PPIL3, PPIL4, PPIL6, PPWD1, PPIAL4G, PTGDS, ERP29, ERP27, PDILT, P4HA3, PLOD1, PLOD2, PLOD3, AGR2, PSMG1, QSOX1, QSOX2, ERLEC1, TUSC3, TXN, PRDX4, FAXC, TXNDC16, TMX4, PDRG1, PFDN1, PFDN2, PFDN4, PFDN5, PFDN6, UXT, VBP1, CRYAA, CRYAB, HSPB1, HSPB2, HSPB3, HSPB6, HSPB7, HSPB8, ODF1, NAPC5, NAPC7, APPBP2, ASPH, P3H2, ST13P5, BBS4, CABIN1, CDC16, CDC23, CDC27, NOT10, CRNKL1, CTR9, EMC2, FICD, GPSM1, GPSM2, GTF3C3, IFIT1, IFIT2, IFIT3, IFIT5, FT88, KLC1, KLC2, KLC3, KLC4, LONRF1, LONRF3, LRP2BP, NASP, NCF2, NOXA1, OGT, P4HA1, P4HA2, PDCD11, PEX5, PEX5L, PRPF6, RAPSN, SH3TC1, SH3TC2, SPAG1, SRP68, SRP72, TANC1, TMTC1, TMTC2, TMTC3, TMTC4, TTC12, TTC13, TTC14, TTC16, TTC17, TTC19, TTC21A, TTC21B, TTC22, TTC23, TTC24, TTC25, TTC26, TTC27, TTC28, TTC29, TTC3, TTC30A, TTC30B, TTC32, TTC33, TTC37, TTC39A, TTC39B, TTC6, TTC7A, TTC7B, TTC8, TTC9, TTC9B, TTC9C, UTY, WDTC1, XAB2, ZC3H7B, ZFC3H1, ZMYND12, AHSA2, DYX1C1, TOMM70A, NUDC, PIH1D1, TSC1, RUVBL2, CHORDC1, RUVBL1, DNAJB3, BBS12, CCT8L1P, HSPA7, HSP90AA2, BAG6, FKBP9L, ERO1L, ERO1LB, PPIAL4A, SDCCAG10, SELS, DERL1, HSPB11, URI1, HSPB9, CFAP70, IFIT1B, KDM6A, TONSL, and/or TRAPPC12.

In some embodiments, the one or more molecular chaperone is provided by expression from a DNA sequence encoding the molecular chaperone included in each of the DNA constructs. In some embodiments, the one or more molecular chaperone sequences included in the DNA constructs can encode molecular chaperones comprising GroE, GroEL, MopA, GroES, MopB, Hsp33, Hsp60, Hsp70, Hsp90, Hsp100, HtpG, ClpB, DnaK, IbpA, IbpB, thioredoxin A (TrxA), small ubiquitin-related modifier (SUMO), Trigger Factor (TF), protein disulfide isomerase (PDI), DsbA, and DsbC. In some embodiments, the one or more molecular chaperone sequences can encode sequences comprising sequences encoded in the heat shock loci (hsl) on the E. coli chromosome. In some embodiments, the one or more molecular chaperone sequences can encode sequences comprising TrxA. In some embodiments, the one or more molecular chaperone sequences can encode sequences comprising DsbA.

In some embodiments, the one or more molecular chaperone is added to the cell-free transcription/translation environment by the addition of one or more DNA sequences encoding the molecular chaperone or by the addition of the molecular chaperone itself to the transcription/translation environment. In some embodiments, the one or more molecular chaperone sequences added as a protein or encoded by a DNA nucleic acid is GroE, GroEL, MopA, GroES, MopB, Hsp33, Hsp60, Hsp70, Hsp90, Hsp100, HtpG, ClpB, DnaK, IbpA, IbpB, thioredoxin A (TrxA), small ubiquitin-related modifier (SUMO), Trigger Factor (TF), protein disulfide isomerase (PDI), DsbA, or DsbC. In some embodiments, the one or more molecular chaperone sequences added as a protein or encoded by a DNA nucleic acid is TrxA. In some embodiments, the one or more molecular chaperone added as a protein or encoded by a DNA nucleic acid is DsbA.

The one or more molecular chaperone sequences can range in size. For example, Hsp33 is a chaperone protein with a molecular weight of 32.9 kDa protein and trxA is another chaperone protein with a molecular weight of 11.25 kDa. In some embodiments, the one or more molecular chaperones can be of a molecular weight between 10 kDa and 100 KkDa, such as 10 kDa to 50 kDa. In some embodiments, the molecular weight is at about 10 kDa, 20 kDa, 30 kDa, 40 kDa, 50 kDa, 60 kDa, 70 kDa, 80 kDa, 90 kDa, and 100 kDa, or any value between any of the foregoing. In some embodiments, the one or more molecular chaperones can be of a molecular weight that independently is 10 kDa, 15 kDa, 20 kDa, 25 kDa, 30 kDa, 35 kDa, 40 kDa, 45 kDa, 50 kDa, 55 kDa, 60 kDa, 65 kDa, 70 kDa, 75 kDa, and 80 kDa, or any value between any of the foregoing. In some embodiments, the one or more molecular chaperones can be of a molecular weight that is independently 10 kDa, 12 kDa, 15 kDa, 18 kDa, 20 kDa, 22 kDa, 25 kDa, 28 kDa, 30 kDa, 32 kDa, 35 kDa, 38 kDa, 40 kDa, 42 kDa, 45 kDa, 48 kDa, 50 kDa, 52 kDa, 55 kDa, 58 kDa, 60 kDa, 62 kDa, 65 kDa, 68 kDa, 70 kDa, 72 kDa, 75 kDa, 78 kDa, and 80 kDa, or any value between any of the foregoing.

In some embodiments, the sequences encoding a molecular chaperone encode one or more molecular chaperones with a percent sequence identity of about at least 30%, 35%, 40%, 45%, 50%, 55%, 60%, 65%, 70%, 75%, 80%, 85%, 90%, 95%, 96%, 97%, 98%, 99%, 99.5%, or about 100% to a known molecular chaperone sequence. In some embodiments, the nucleotide sequences encode one or more molecular chaperones with variant sequences comprising 1 mismatch, 2 mismatches, 3 mismatches, 4 mismatches, 5 mismatches, 6 mismatches, 7 mismatches, 8 mismatches, 9 mismatches, or 10 mismatches between the variant sequences and reference molecular chaperone sequences (e.g., known in art). In some embodiments, the nucleotide sequences encode one or more molecular chaperones with variant sequences comprising 11 mismatches, 12 mismatches, 13 mismatches, 14 mismatches, 15 mismatches, 16 mismatches, 17 mismatches, 18 mismatches, 19 mismatches, or 20 mismatches between the variant sequence and the reference molecular chaperone sequences (e.g., known in art). In some embodiments, the nucleotide sequences encode one or more molecular chaperones with variant sequences comprising about between 1 and 500, between 10 and 450, between 20 and 400, between 30 and 350, between 40 and 300, between 50 and 250, between 60 and 200, between 70 and 150, and between 80 and 100 mismatches between the variant sequence and the reference molecular chaperone sequence (e.g., known in art).

In some embodiments, the amino acids of one or more molecular chaperones have a percent sequence identity of about at least 30%, 35%, 40%, 45%, 50%, 55%, 60%, 65%, 70%, 75%, 80%, 85%, 90%, 95%, 96%, 97%, 98%, 99%, 99.5%, or about 100% to a known molecular chaperone amino acid sequence. In some embodiments, the amino acids of one or more molecular chaperones comprise variant amino acids which comprise 1 mismatch, 2 mismatches, 3 mismatches, 4 mismatches, 5 mismatches, 6 mismatches, 7 mismatches, 8 mismatches, 9 mismatches, or 10 mismatches between the variant amino acid and the reference (e.g., known in art) molecular chaperone amino acid sequence. In some embodiments, the amino acids of one or more molecular chaperones comprise variant amino acids which comprise 11 mismatches, 12 mismatches, 13 mismatches, 14 mismatches, 15 mismatches, 16 mismatches, 17 mismatches, 18 mismatches, 19 mismatches, or 20 mismatches between the variant amino acid and the reference (e.g., known in art) molecular chaperone amino acid sequence. In some embodiments, the amino acids of one or more molecular chaperones comprise variant amino acids comprising about between 1 and 500, between 10 and 450, between 20 and 400, between 30 and 350, between 40 and 300, between 50 and 250, between 60 and 200, between 70 and 150, and between 80 and 100 mismatches between the variant amino acid and the reference molecular chaperone sequences (e.g., known in art).

In some embodiments, the sequences encode one or more molecular chaperones with a percent sequence identity of about at least 30%, 35%, 40%, 45%, 50%, 55%, 60%, 65%, 70%, 75%, 80%, 85%, 90%, 95%, 96%, 97%, 98%, 99%, 99.5%, or about 100% to trxA (i.e., Thioredoxin 1; SEQ ID NO: 11). In some embodiments, the nucleotide sequences encode one or more molecular chaperones with variant sequences comprising 1 mismatch, 2 mismatches, 3 mismatches, 4 mismatches, 5 mismatches, 6 mismatches, 7 mismatches, 8 mismatches, 9 mismatches, or 10 mismatches between the variant sequences and trxA (i.e., Thioredoxin 1; SEQ ID NO: 11). In some embodiments, the nucleotide sequences encode one or more molecular chaperones with variant sequences comprising 11 mismatches, 12 mismatches, 13 mismatches, 14 mismatches, 15 mismatches, 16 mismatches, 17 mismatches, 18 mismatches, 19 mismatches, or 20 mismatches between the variant sequence and trxA (i.e., Thioredoxin 1; SEQ ID NO: 11). In some embodiments, the nucleotide sequences encode one or more molecular chaperones with variant sequences comprising about between 1 and 500, between 10 and 450, between 20 and 400, between 30 and 350, between 40 and 300, between 50 and 250, between 60 and 200, between 70 and 150, and between 80 and 100 mismatches between the variant sequence and trxA (i.e., Thioredoxin 1; SEQ ID NO: 11).

In some embodiments, the amino acids of one or more molecular chaperones have a percent sequence identity of about at least 30%, 35%, 40%, 45%, 50%, 55%, 60%, 65%, 70%, 75%, 80%, 85%, 90%, 95%, 96%, 97%, 98%, 99%, 99.5%, or about 100% to trxA (i.e., Thioredoxin 1; SEQ ID NO: 13) amino acid sequence. In some embodiments, the amino acids of one or more molecular chaperones comprise variant amino acids which comprise 1 mismatch, 2 mismatches, 3 mismatches, 4 mismatches, 5 mismatches, 6 mismatches, 7 mismatches, 8 mismatches, 9 mismatches, or 10 mismatches between the variant amino acid and trxA (i.e., Thioredoxin 1; SEQ ID NO: 13) amino acid sequence. In some embodiments, the amino acids of one or more molecular chaperones comprise variant amino acids which comprise 11 mismatches, 12 mismatches, 13 mismatches, 14 mismatches, 15 mismatches, 16 mismatches, 17 mismatches, 18 mismatches, 19 mismatches, or 20 mismatches between the variant amino acid and trxA (i.e., Thioredoxin 1; SEQ ID NO: 13) amino acid sequence. In some embodiments, the amino acids of one or more molecular chaperones comprise variant amino acids comprising about between 1 and 500, between 10 and 450, between 20 and 400, between 30 and 350, between 40 and 300, between 50 and 250, between 60 and 200, between 70 and 150, and between 80 and 100 mismatches between the variant amino acid and trxA (i.e., Thioredoxin 1; SEQ ID NO: 13).

In some embodiments, the sequences encode one or more molecular chaperones with a percent sequence identity of about at least 30%, 35%, 40%, 45%, 50%, 55%, 60%, 65%, 70%, 75%, 80%, 85%, 90%, 95%, 96%, 97%, 98%, 99%, 99.5%, or about 100% to DsbA (i.e., Thiol:disulfide interchange protein; SEQ ID NO: 12). In some embodiments, the nucleotide sequences encode one or more molecular chaperones with variant sequences comprising 1 mismatch, 2 mismatches, 3 mismatches, 4 mismatches, 5 mismatches, 6 mismatches, 7 mismatches, 8 mismatches, 9 mismatches, or 10 mismatches between the variant sequences and DsbA (i.e., Thiol:disulfide interchange protein; SEQ ID NO: 12). In some embodiments, the nucleotide sequences encode one or more molecular chaperones with variant sequences comprising 11 mismatches, 12 mismatches, 13 mismatches, 14 mismatches, 15 mismatches, 16 mismatches, 17 mismatches, 18 mismatches, 19 mismatches, or 20 mismatches between the variant sequence and DsbA (i.e., Thiol:disulfide interchange protein; SEQ ID NO: 12). In some embodiments, the nucleotide sequences encode one or more molecular chaperones with variant sequences comprising about between 1 and 500, between 10 and 450, between 20 and 400, between 30 and 350, between 40 and 300, between 50 and 250, between 60 and 200, between 70 and 150, and between 80 and 100 mismatches between the variant sequence and DsbA (i.e., Thiol:disulfide interchange protein; SEQ ID NO: 12).

In some embodiments, the amino acids of one or more molecular chaperones have a percent sequence identity of about at least 30%, 35%, 40%, 45%, 50%, 55%, 60%, 65%, 70%, 75%, 80%, 85%, 90%, 95%, 96%, 97%, 98%, 99%, 99.5%, or about 100% to DsbA (i.e., Thiol:disulfide interchange protein; SEQ ID NO: 14) amino acid sequence. In some embodiments, the amino acids of one or more molecular chaperones comprise variant amino acids which comprise 1 mismatch, 2 mismatches, 3 mismatches, 4 mismatches, 5 mismatches, 6 mismatches, 7 mismatches, 8 mismatches, 9 mismatches, or 10 mismatches between the variant amino acid and DsbA (i.e., Thiol:disulfide interchange protein; SEQ ID NO: 14) amino acid sequence. In some embodiments, the amino acids of one or more molecular chaperones comprise variant amino acids which comprise 11 mismatches, 12 mismatches, 13 mismatches, 14 mismatches, 15 mismatches, 16 mismatches, 17 mismatches, 18 mismatches, 19 mismatches, or 20 mismatches between the variant amino acid and DsbA (i.e., Thiol:disulfide interchange protein; SEQ ID NO: 14) amino acid sequence. In some embodiments, the amino acids of one or more molecular chaperones comprise variant amino acids comprising about between 1 and 500, between 10 and 450, between 20 and 400, between 30 and 350, between 40 and 300, between 50 and 250, between 60 and 200, between 70 and 150, and between 80 and 100 mismatches between the variant amino acid and DsbA (i.e., Thiol:disulfide interchange protein; SEQ ID NO: 14).

For further details on molecular chaperones see, e.g., Shemesh, N., Jubran, J., Dror, S. et al. The landscape of molecular chaperones across human tissues reveals a layered architecture of core and variable chaperones. Nat Commun 12, 2180 (2021). doi.org/10.1038/s41467-021-22369-9; Lund P A. Microbial molecular chaperones. Adv Microb Physiol. 2001; 44:93-140. doi: 10.1016/s0065-2911(01) 44012-4. PMID: 11407116; Chuang S E, Blattner F R. Characterization of twenty-six new heat shock genes of Escherichia coli. J Bacteriol. 1993 August; 175 (16): 5242-52. doi: 10.1128/jb.175.16.5242-5252.1993. PMID: 8349564; PMCID: PMC204992, which are incorporated by reference herein in their entireties for all purposes.

### 3. Replication Initiator Proteins

The in vitro library selection system described herein comprises DNA constructs that encode a DRP of interest and a peptide with cis binding activity for said construct. In some embodiments, the in vitro library selection system described herein comprises DNA constructs that encode a DRP of interest, one or more molecular chaperones, and a peptide with cis binding activity for said construct. In other embodiments, the one or more molecular chaperone is provided to the cell-free environment separately, and the in vitro library selection system described herein comprises DNA constructs that encode a DRP of interest and a peptide with cis binding activity for said construct. In some embodiments, the peptide with cis binding activity for said construct is a replication initiator protein. The cis activity of this in vitro screening is defined by the ability of the peptide (e.g., replication initiator protein) with cis binding activity to bind to the DNA from which the peptide was produced. Hence, replication initiation proteins (Rep proteins) are a class of proteins that mediate the initiation of plasmid DNA replication typically in bacteria. For example, the peptide transcribed and translated from a DNA and an RNA transcript, respectively, will bind to the DNA transcript. In some embodiments, an expressed DNA replication initiator protein, for example RepA, binds to its expression construct.

In some embodiments, the DNA replication initiator protein is RepA or a functional variant thereof. RepA is a protein that initiates plasmid DNA replication by binding to specific origin of replication (ori) sequences, also referred to as iterons, that are present in the DNA construct. In the context of the present disclosure, the DNA replication initiator protein is expressed as a fusion to a disulfide-rich protein (DRP), thereby providing a physical linkage between the DRP and the cognate DNA that encoded it. In provided embodiments, a “target sequence” in a provided DNA construct is a a non-coding DNA sequence within the DNA construct that is specifically recognized by the DNA replication initiator protein. For RepA, the target sequence comprises one or more ori or iteron sequences that form the RepA binding site. These non-coding DNA elements are not translated into protein but instead serve as cis-acting recognition sites for the initiator protein. When expressed, the RepA-DRP fusion protein, optionally also including a molecular chaperone as described, binds in cis to the origin of replication sequence present on the same DNA construct, thereby tethering the protein to its encoding nucleic acid. This molecular linkage underlies the cis-display system and enables construction of libraries in which each DRP is physically associated with its own encoding DNA sequence.

Any DNA element which allows the peptide encoding the DRP to bind specifically to the DNA molecule which encoded it may be used as a DNA element replication initiator protein. One example of a suitable DNA element is RepA (e.g., SEQ ID NO: 8). The DNA element, RepA, and its genetic control elements, ORI (e.g., SEQ ID NO: 10) and CIS (e.g., SEQ ID NO: 9), allow the linking of the expressed peptide or protein to its own DNA sequence. For example, the RNA polymerase is paused by loops in the 5′ CIS sequence prior to the rho dependent termination of transcription. This delay allows nascent RepA polypeptide emerging from translating ribosomes to bind transiently to CIS, which in turn directs the protein to bind to the adjacent ORI site. The action of RepA therefore allows the encoded binding peptide to bind to the DNA target sequence in the construct from which it was produced. The cis element is required for cis activity of the RepA protein (for further description refer to Praszkier and Pittard 1999 J. Bacteriol. 181:2765-2772). The cis DNA element should therefore also be located 3′ in the DNA construct to the DNA encoding the RepA sequence. On reaching the cis sequence, the RNA polymerase will be paused, allowing the encoded RepA protein to bind the DNA target sequence.

Numerous plasmids include sequences encoding RepA and cis DNA elements. The RepA sequence and cis DNA element present in a DNA construct of the invention may be derived from the same plasmid strain or may be derived from different plasmid strains. RepA proteins are well-characterized initiators encoded by a variety of plasmid incompatibility (Inc) groups. RepA proteins function by binding to specific origin of replication (oriV) sequences on their cognate plasmids, thereby recruiting host replication machinery and promoting stable plasmid maintenance. In general, plasmids belonging to the same incompatibility group encode homologous RepA proteins that utilize overlapping replication mechanisms, and thus are unable to stably coexist in the same host cell. By contrast, plasmids from different incompatibility groups encode distinct RepA proteins, enabling co-residence of multiple plasmids within a single bacterial strain.

Suitable RepA proteins and sequences and cis DNA elements include those of the IncI complex plasmids (for example, R64 or ColIb-P9) or the IncF, IncB, IncK, IncZ and IncL/M plasmids, which are distantly related at the DNA level, but which control plasmid replication through the action of the cis acting RepA protein (see e.g., Nikoletti et al. 1986 J. Bacteriol. 170:1311-1318 and Prazkier J. et al. 1991 J. Bacteriol. 173:2393-2397). Specific plasmids which may be used to provide these sequences include the R1 plasmid of the IncII incompatibility group and the incB plasmid pMU720 (described by Praskier J. & Pittard J. 1999 Role of CIS in replication of an IncB plasmid. J. Bacteriol. 181:2765-2772). Typically, the cis element (e.g., SEQ ID NO: 9) is 150 to 200 nucleotides in length. Shorter or larger sequences may be used, so long as the sequence maintains the ability to interact with RepA and display cis activity. Minor variations, such as substitutions or deletions within the cis sequence are also contemplated such as modifications at 1, 5, 10, and up to 20 nucleotides within the wildtype cis sequence.

The RepA protein used in accordance with the present invention may also comprise a fragment or variant of RepA (e.g., SEQ ID NO: 8), so long as such variant or fragment of RepA maintains the ability to bind to the selected ori sequence. Such variant or fragment of RepA may include substitutions, for example, at 1, 2, 3 up to 20 amino acids within the RepA sequence so long as such variants maintain the ability to bind to the ori (e.g., SEQ ID NO: 10) sequence. A suitable fragment of RepA is an ori binding sequence of RepA. Ori sequences include those which are present in wild type plasmids as described above. Typically, such an ori sequence is 170 to 220 nucleotides in length. Fragments and variants of wild type ori sequences may also be used, so long as such ori sequences maintain the ability to be recognized by RepA. Further cis acting members of the RepA protein family can be used. For example, the RepA family of proteins is found on plasmids with a broad host range i.e., one RepA plasmid may be found in different bacterial species. Isolation of a RepA family plasmid from (for example) a thermophilic, sulphatic, halophilic, or acidophilic bacterium, would provide RepA-cis-ori DNA that could be used under the current invention at elevated temperatures or extremes of salt, pH, or sulfur concentrations. Such members of the RepA family would be advantageous in isolating library members that can bind to target molecules under such extreme conditions. Suitable ori sequences for use in combination with selected RepA proteins can readily be determined by monitoring for the interaction of RepA with such an ori sequence.

The RepA family of proteins is used herein by way of example, not limitation. Other unrelated non-covalently binding cis acting DNA binding proteins could be used in this invention. The compatibility of a RepA sequence from a plasmid with a cis sequence from another plasmid can be readily determined by monitoring for the interaction of RepA with the selected cis sequence.

In embodiments, the DNA construct also includes (i) a sequence encoding a DNA replication initiator protein such as RepA, (ii) a cis DNA element located downstream of the RepA coding sequence, and (iii) a DNA sequence comprising a target sequence recognized by RepA. The target sequence is typically an origin of replication (ori), which provides the direct binding site for RepA. The cis DNA element is a non-coding sequence that functionally separates RepA from the origin of replication and enforces cis-acting binding, ensuring that RepA associates with the ori sequence present on the same DNA construct. Together, the cis DNA element and the ori enable the expressed RepA fusion protein, for example a disulfide-rich protein (DRP) fused to RepA, to bind directly and specifically to the ori sequence on the same construct, thereby establishing cis linkage.

Replication initiation proteins in general recognize characteristic origin of replication (ori) sequences present in their cognate plasmids. The ori serves as the prototypical target sequence and typically comprises one or more iterons, which are short, repeated DNA motifs that provide docking sites for the initiator protein. Binding of RepA to the iterons within the ori is the critical molecular interaction that directs initiation of plasmid replication and stabilizes plasmid inheritance in bacterial cells.

For purposes of the provided embodiments, the target sequence included within each DNA construct of the cis-display library is selected such that it is recognized by the DNA replication initiator protein encoded by the same construct. In certain embodiments, the DNA replication initiator protein is RepA, and the target sequence comprises an ori sequence recognized by RepA. In some embodiments, the ori comprises one or more iterons that bind RepA with high specificity. In other embodiments, variants of RepA that retain the ability to bind their cognate target sequence may be employed, and the target sequence may likewise be modified, truncated, or engineered so long as sufficient binding activity is retained.

In some embodiments the target sequence to which the DNA replication initiator protein binds is an ori, typically comprising iterons, and the DNA construct further comprises a cis DNA element positioned downstream of repA and upstream of the ori. While not itself the direct binding site for RepA, the cis DNA element enforces cis-acting interactions, ensuring that the RepA fusion protein binds to the ori sequence present on the same construct. Together, the ori (target sequence) and the cis DNA element provide a robust genotype-phenotype linkage in the cis-display system.

In some embodiments, each construct comprises a DNA sequence encoding a DRP to be expressed as a library member DRP and each contains an appropriate promoter, ribosome binding site and start codon, the one or more chaperone genes, one or more linker sequences, translation start and stop signals, the replication initiator protein sequence (e.g. SEQ ID NO: 8) followed by the CIS (e.g., SEQ ID NO: 9) and ORI (e.g., SEQ ID NO: 10) non-coding DNA elements required for CIS activity. In some embodiments, the CIS DNA element will be located 3′ in the DNA construct to the library member DRP and to the peptide or protein (e.g., RepA) capable of binding to the DNA target sequence. This means that these sequences may be transcribed and translated before the RNA polymerase reaches the cis acting sequence.

A DNA element that directs cis-activity may be provided in the DNA construct together with the DNA encoding a peptide that interacts with that cis element. For example, in the case of the cis element for RepA (e.g., SEQ ID NO: 8), DNA encoding a fragment of the RepA sequence comprising at least 20 amino acids from the C terminal of RepA may be provided along with the cis DNA element.

By incorporating both a cis DNA element and a target sequence into the DNA construct, the provided embodiments ensure that the expressed RepA-protein fusion binds back specifically to the DNA construct from which it was transcribed. The cis DNA element enforces cis-acting linkage, preventing RepA from binding ori sequences on other constructs in the mixture, while the ori provides the direct RepA docking site. This arrangement provides a robust genotype-phenotype linkage, as the protein fusion (for example, a disulfide-rich protein fused to RepA) is physically tethered to its encoding DNA construct via specific binding of RepA to the ori. This linkage underpins the cis-display library system, enabling a stable and retrievable pool of DNA:protein fusions for selection and enrichment.

In some embodiments, the target sequence is derived from the origin of replication (ori) of the IncFII plasmid R1. The R1 plasmid ori is a well-characterized replication origin belonging to the IncF incompatibility group and includes a series of iterated DNA motifs (iterons) that are specifically recognized and bound by the RepA protein of IncFII plasmids. In some embodiments, a cis DNA element located between repA and ori is also present, thereby enforcing cis-acting RepA-ori interactions and maintaining genotype-phenotype linkage in the display system. For example, the target sequence is the ori of the IncFII plasmid R1 and the cis DNA element comprises a sequence derived from the cis-acting region of the IncFII plasmid R1. In some embodiments, the DNA construct comprises the sequence encoding RepA, the cis DNA element and the ori DNA of the IncFII plasmid R1, together with a sequence encoding the DRP and optionally a sequence encoding one or more molecular chaperone. In some embodiments, the DNA construct comprises the sequence encoding RepA set forth in SEQ ID NO: 8, the cis DNA element set forth in SEQ ID NO:9, the ori DNA sequence set forth in SEQ ID NO: 10, together with a sequence encoding the DRP and optionally a sequence encoding one or more molecular chaperone.

The CIS binding between the replication initiator protein and the DNA transcript is a non-covalent interaction. Non-covalent binding refers to an association that may be disrupted by methods well known to those skilled in the art, such as the addition of an appropriate solvent, or a change in ionic conditions, for example, the addition of low pH glycine or high pH triethylamine. The peptide or protein (e.g., disulfide rich protein) encoded by the construct remains associated with the DNA construct sufficiently to allow the protein: DNA complex to be separated from the resulting mixture.

For example, the association between the encoded protein (i.e., DRP) and its DNA may have a half-life of up to 30 minutes, up to 45 minutes, up to one hour, up to 2 hours, up to 6 hours or up to 12 hours. The screening methods of the invention may therefore be carried out immediately after construction of the library, or later, for example up to one, up to two, up to six, up to twelve hours or up to twenty-four hours or more than twenty-four hours later.

For further description see e.g., U.S. Patent Number U.S. Pat. No. 8,679,781B2 and Odegrip R, Coomber D, Eldridge B, Hederer R, Kuhlman P A, Ullman C, FitzGerald K, McGregor D. CIS display: In vitro selection of peptides from libraries of protein-DNA complexes. Proc Natl Acad Sci USA. 2004 Mar. 2; 101(9):2806-10. doi: 10.1073/pnas.0400219101. Epub 2004 Feb. 23. PMID: 14981246; PMCID: PMC365701, which are incorporated by reference herein in their entireties for all purposes.

### B. CIS Display Libraries of DNA Constructs

The methods provided herein allow for an in vitro library selection system (CIS display) that encodes a diverse library of disulfide rich proteins (DRPs). The DRP library comprises DRPs characterized by two or more disulfide bonds. A plurality of DNA constructs, encoding a plurality of different DRPs and optionally one or more molecular chaperones form a DNA library of the invention. In some embodiments, a DNA construct encodes a library member DRP. In some embodiments, a DNA library is therefore a population of DNA constructs. Expressing such a library of DNA constructs results in the non-covalent binding of individual encoded proteins to the DNA which encoded them and from which they have been transcribed and translated, in the presence of many other DNA molecules that encode other members of the library. The sequence encoding the library member present in a particular encoded protein will therefore be present in the DNA which is bound to that protein. This process therefore links the library member peptide, in a biologically active form (usually having a binding activity) to the specific library member DNA sequence encoding that DRP, allowing selection of DRPs of interest, for example due to a particular binding activity, and subsequent isolation and identification of the DNA encoding that library member DRP. In some embodiments, a DNA library of the invention will contain a plurality of DNA constructs. In some embodiments, a DNA library is a CIS Display library that contains a plurality of DNA:protein fusions that are generated by expressing a plurality of the DNA constructs in a prokaryotic cell-free transcription/translation environment. In some embodiments, protein fusions comprise the DRP, optionally the molecular chaperone, and the DNA replication initiator protein, in which the protein fusion binds, via the DNA replication initiator protein, to the target sequence in the DNA construct from which it was transcribed, thereby linking the protein fusion to the DNA construct to produce the DRP CIS display library comprising a plurality of DNA:protein fusions.

In some embodiments, the DNA constructs comprise DNA sequences encoding a DRP, one or more molecular chaperone, a replication initiator protein, and a target sequence for the DNA replication initiator protein. In some aspects, the DNA replication initiator protein can non-covalently bind to the target sequence. A plurality of DNA constructs is provided each encoding a library member to provide a plurality of different library members. In some embodiments, a DNA library will contain at least 10⁴ discrete DNA constructs. In some embodiments, a DNA library will contain at least 10⁶ discrete DNA constructs. In some embodiments, a DNA library will contain at least 10⁸ discrete DNA constructs. For example, a DNA library may contain more than 10⁶, more than 10⁸, more than 10¹⁰, more than 10¹², or more than 10¹⁴ discrete DNA constructs.

In some embodiments, the provided libraries are CIS display libraries. In some embodiments, the CIS display library comprises a plurality of DNA:protein fusions each containing a protein fusion of a DRP, one or more molecular chaperone, a replication initiator protein (e.g., RepA) that is linked in cis to the target sequence of the RepA, such as via DNA elements CIS and ORI. In some embodiments, the CIS display library is produced through an in vitro transcription translation reaction to express the DRP-chaperone-replication initiator protein fusion. The protein fusion binds, via the DNA replication initiator protein, to the target sequence in the DNA construct from which it was transcribed to form the plurality of DNA:protein fusions. A plurality of DNA:protein fusions is provided each encoding a library member to provide a plurality of different library members. In some embodiments, a CIS display library contains at least 10⁴ DNA:protein fusions, such as 10⁶ to 10¹⁴ different DNA:protein fusions or 10¹⁰ to 10¹⁴ different DNA:protein fusions.

In some embodiments, a CIS display library is produced by a DNA construct encoding fusion of a DRP to one or more chaperone as described herein, such as a CDR3 knob peptide and a trxA chaperone fusion.

In some embodiments, a library of DNA constructs encoding DRPs in-frame with the molecular chaperone and replication initiator protein (e.g., RepA) comprises one or more DNA constructs expressing chaperone-DRP-RepA.

In some embodiments, the DNA constructs comprise DNA sequences encoding a DRP, a replication initiator protein, and a target sequence for the DNA replication initiator protein. In some aspects, the DNA replication initiator protein can non-covalently bind to the target sequence. A plurality of DNA constructs is provided each encoding a library member to provide a plurality of different library members. In some embodiments, a DNA library will contain at least 10⁴ discrete DNA constructs. In some embodiments, a DNA library will contain at least 10⁶ discrete DNA constructs. In some embodiments, a DNA library will contain at least 10⁸ discrete DNA constructs. For example, a DNA library may contain more than 10⁶, more than 10⁸, more than 10¹⁰, more than 10¹², or more than 10¹⁴ discrete DNA constructs.

In some embodiments, the provided libraries are CIS display libraries. In some embodiments, the CIS display library comprises a plurality of DNA:protein fusions each containing a protein fusion of a DRP and a replication initiator protein (e.g., RepA) that is linked in cis to the target sequence of the RepA, such as via DNA elements CIS and ORI. In some embodiments, the CIS display library is produced through an in vitro transcription translation reaction to express the DRP-replication initiator protein fusion. The protein fusion binds, via the DNA replication initiator protein, to the target sequence in the DNA construct from which it was transcribed to form the plurality of DNA:protein fusions. A plurality of DNA:protein fusions is provided each encoding a library member to provide a plurality of different library members. In some embodiments, a CIS display library contains at least 10⁴ DNA:protein fusions, such as 10⁶ to 10¹⁴ different DNA:protein fusions or 10¹⁰ to 10¹⁴ different DNA:protein fusions.

In some embodiments, the polypeptides for CIS display include disulfide rich proteins (DRPs). In some embodiments, DRPs comprise a disulfide-rich region.

In some embodiments, the nucleotide sequence of an encoded DRP encodes a peptide having a random amino acid composition. In some embodiments, the DRP encodes a sequence encoding a random amino acid of varying length compared to the other DRPs encoded in other constructs. In some embodiments, the DRPs encodes a sequence encoding random amino acid of the same length compared to the other DRPs encoded in other constructs.

In some embodiments, the nucleotide sequences of a library of DRPs encode peptides having random amino acid compositions. In some embodiments, the library of DRPs encode sequences encoding random amino acids of varying lengths. In some embodiments, the library of DRPs encode sequences encoding random amino acids of the same length.

In some embodiments, a library of DRPs encode peptides having random amino acid compositions. In some embodiments, the random amino acids are of varying length. In some embodiments, the random amino acids are of the same length.

In some embodiments, the nucleotide sequences of a library of DRPs encode known peptides or fragments or variants thereof. In some embodiments, the nucleotide sequences of a library of DRPs encode known peptides or fragment or variants thereof of varying length. In some embodiments, the nucleotide sequences of a library of DRPs encode known peptides or fragments or variants thereof of the same length.

In some embodiments, the nucleotide sequences of a library of DRPs encode known peptides or fragments or variants thereof. In some embodiments, the library of DRPs encode sequences encoding known peptides or fragments or variants thereof of varying length. In some embodiments, the library of DRPs encode sequences encoding known peptides or fragments or variants thereof of the same length.

In some embodiments, the nucleotide sequences of a library of DRPs encode fragments comprising about 30%, 35%, 40%, 45%, 50%, 55%, 60%, 65%, 70%, 75%, 80%, 85%, 90%, 95%, 96%, 97%, 98%, 99%, 99.5%, or about 100% of a sequence encoding a known peptide (e.g., reference peptide).

In some embodiments, the nucleotide sequences of a library of DRPs encode a variant sequence comprising about 30%, 35%, 40%, 45%, 50%, 55%, 60%, 65%, 70%, 75%, 80%, 85%, 90%, 95%, 96%, 97%, 98%, 99%, 99.5%, or about 100% of a sequence encoding a known (e.g., reference) peptide. In some embodiments, the nucleotide sequences of a library of DRPs encode a plurality of variant sequences comprising 1 mismatch, 2 mismatches, 3 mismatches, 4 mismatches, 5 mismatches, 6 mismatches, 7 mismatches, 8 mismatches, 9 mismatches, or 10 mismatches between a member of the plurality of variant sequences and the reference peptide sequence. In some embodiments, the nucleotide sequences of a library of DRPs encode a plurality of variant sequences comprising 11 mismatches, 12 mismatches, 13 mismatches, 14 mismatches, 15 mismatches, 16 mismatches, 17 mismatches, 18 mismatches, 19 mismatches, or 20 mismatches between the variant sequence and the reference peptide sequence. In some embodiments, the nucleotide sequences of a library of DRPs encode a plurality of variant sequences comprising about between 1 and 500, between 10 and 450, between 20 and 400, between 30 and 350, between 40 and 300, between 50 and 250, between 60 and 200, between 70 and 150, and between 80 and 100 mismatches between the variant sequence and the reference peptide sequence.

In some embodiments, the amino acids of a library of DRPs code for fragments comprising about 20%, 25%, 30%, 35%, 40%, 45%, 50%, 55%, 60%, 65%, 70%, 75%, 80%, 85%, 90%, 95%, 96%, 97%, 98%, 99%, 99.5%, or about 100% of the amino acids coding for a known reference peptide.

In some embodiments, the amino acids of a library of DRPs code for variant peptides comprising about 20%, 25%, 30%, 35%, 40%, 45%, 50%, 55%, 60%, 65%, 70%, 75%, 80%, 85%, 90%, 95%, 96%, 97%, 98%, 99%, 99.5%, or about 100% of the amino acids coding for a known reference peptide.

In some embodiments, the polypeptides for display are disulfide rich proteins. In some embodiments, the DRPs are synthetic. In some embodiments, the DRPs are semisynthetic. In some embodiments, the DRPs are natural. In some embodiments, the DRPs are natural, semisynthetic, and synthetic. In some embodiments, the DRPs are natural, and synthetic. In some embodiments, the natural, semisynthetic, and/or synthetic DRPs include all or a portion of an antibody. In some embodiments, the natural, semisynthetic, and/or synthetic DRPs include all or a portion of a bovine antibody, e.g., an ultralong CDR3 knob. In some embodiments, the natural, semisynthetic, and/or synthetic DRPs include all or a portion of a single-chain variable fragment, e.g., scFv. In some embodiments, the natural, semisynthetic, and/or synthetic DRP is a modified knottin or an inhibitor cystine knot (ICK). In some embodiments, the natural, semisynthetic, and/or synthetic DRP is a modified cyclotide. In some embodiments, the modified cyclotide includes an ultralong CDR3 knob sequence, e.g., of a cow.

In some embodiments, the DRPs for display contain a variable heavy region containing the CDR-H3 and a variable light region. Particular formats include single chain formats, such as a single chain variable fragment (scFv). In other embodiments, the DRP for display is a smaller peptide of 25-70 amino acids, such as 40-70 amino acids, that is a knob peptide. Exemplary DRP molecules for display and display libraries are described herein (i.e., Section II.A.1).

In some embodiments, a screening CIS display library produced according to the methods herein comprises a plurality of DNA:protein fusions, wherein each fusion comprises a DRP-chaperone-DNA replication initiator protein bound to the corresponding DNA construct encoding said DRP-chaperone-DNA replication initiator protein sequence. In some embodiments, a screening CIS display library produced according to the methods herein comprises a plurality of DNA:protein fusions, wherein each fusion comprises a DRP-DNA replication initiator protein bound to the corresponding DNA construct encoding said DRP-DNA replication initiator protein sequence. In some aspects, provided herein are methods of producing knob disulfide rich proteins (DRPs) CIS display libraries and screening said libraries for binding of molecules of interest. For further disclosure on knob DRPs that can be used for screening refer to details disclosed herein (i.e., Section II.A.1.a).

In some aspects, provided herein are methods of producing synthetic disulfide rich proteins (DRPs) CIS display libraries and screening said libraries for binding of molecules of interest. In some embodiments, the DRP for display is a synthetic peptide. In some embodiments, the synthetic peptide is a random sequence polypeptide with a cysteine motif and disulfide bonds as described herein, e.g., with 4-20 cysteine residues and 2-10 disulfide bonds. In some embodiments, the synthetic peptide has been selected from a random sequence library for having a cysteine motif and disulfide bonds as described herein, e.g., for having 4-20 cysteine residues and 2-10 disulfide bonds. For further disclosure on synthetic DRPs that can be used for screening refer to details disclosed herein (i.e., Section II.A.1.b).

In some embodiments, the DRP for display is a cyclotide. In some embodiments, the DRP for display is a modified cyclotide, e.g., that has been modified to include an exogenous peptide sequence. In some embodiments, the modified cyclotide includes an ultralong CDR3 knob sequence or a portion thereof, including any as described herein or identified according to the provided methods. For further disclosure on cyclotides that can be used for screening refer to details disclosed herein (i.e., Section II.A.1.c).

In some embodiments, the DRP for display is a single-chain variable fragment (scFv). In some embodiments, the scFv includes a VH region having a cow ultralong CDR3. In some embodiments, the scFv comprises a human scFv. In some embodiments, the scFv comprises a mouse scFv. In some embodiments, the scFv comprises a rat scFv. In some embodiments, the scFv comprises a rabbit scFv. Provided herein are methods of producing scFv DRP CIS display libraries and screening said libraries for binding of molecules of interest. Exemplary scFv DRPs for display libraries are described herein (i.e., Section II.A.1.d).

Any known methods for generating libraries of DNA constructs containing variant polynucleotides and/or polypeptides can be used with the provided methods and constructs to generate CIS display libraries and to select binding proteins from the libraries. The libraries can then be used in screening assays to select binding proteins from the library for any target of interest (e.g., antigen), including, for example, any virus, bacterial, other pathogenic, an immunomodulatory protein (e.g., a checkpoint molecule), or cancer antigen.

Techniques for manipulating nucleic acids, such as those for generating mutation in sequences, subcloning, labeling, probing, sequencing, hybridization and so forth, are described in detail in scientific publications and patent documents. See, for example, Sambrook J, Russell D W (2001) Molecular Cloning: A Laboratory Manual, 3rd ed. Cold Spring Harbor Laboratory Press, New York; Current Protocols in Molecular Biology, Ausubel ed., John Wiley & Sons, Inc., New York (1997); Laboratory Techniques in Biochemistry and Molecular Biology: Hybridization with Nucleic Acid Probes, Part I, Theory and Nucleic Acid Preparation, Tijssen ed., Elsevier, N.Y. (1993).

The DNA constructs comprising multiple DRPs-chaperone-RepA constructs are then ready for in vitro transcription and translation. Alternatively, the DNA constructs comprising multiple DRPs-RepA constructs are then ready for in vitro transcription and translation in combination with addition of one or more molecular chaperone or a nucleic acid encoding one or more molecular chaperone that is added to the prokaryotic cell-free transcription/translation environment.

### C. Expressing a CIS Display Library in a Reduced Acellular Environment

Also provided herein in some embodiments are methods of generating CIS display libraries comprising disulfide rich polypeptides and in some embodiments one or more molecular chaperones. The DRPs that are screened as part of the methods are peptides or proteins (e.g., of 15 to 250 amino acids in length, such as 25 to 70 amino acids in length) that contain 4 or more cysteine residues from which it is desired to produce a disulfide-rich protein comprising two or more disulfide bonds.

In some aspects, the methods involve generating a library of single DNA constructs (e.g., expression cassettes) of a chaperone, DRP and CIS-acting element (RepA) that are joined together by a linker. In some embodiments, the provided methods include performing a coupled in vitro transcription-translation reaction (ITT) in a bacterial lysate to express the CIS display libraries. In some embodiments, the expressed and linked chaperone-DRP-RepA DNA:protein fusion from the library can be screened to assess binding to one or more targets of interest. In some embodiments, the DRP and the chaperone, e.g., bacterial chaperone, are joined by a linker. In some embodiments, the DRP and the chaperone and the CIS-acting element (e.g., RepA) are joined by a linker.

In some aspects, the methods involve generating a library of single DNA constructs (e.g., expression cassettes) of a DRP and CIS-acting element (RepA) that are joined together by a linker. In some embodiments, the provided methods include performing a coupled in vitro transcription-translation reaction (ITT) in a bacterial lysate to express the CIS display libraries. In some embodiments, the expressed and linked DRP-RepA DNA:protein fusion from the library can be screened to assess binding to one or more targets of interest. In some embodiments, the DRP and the CIS-acting element (e.g., RepA) are joined by a linker. In some embodiments, the methods involve further providing a molecular chaperone. In some aspects, the molecular chaperone is provided by expression from a DNA sequence encoding the molecular chaperone included in each of the DNA constructs, and wherein each of the plurality of DNA:protein fusion proteins comprises a DRP, the molecular chaperone, and the DNA replication initiator protein. In some aspects, the molecular chaperone is separately provided as one or one or more DNA sequences encoding a molecular chaperone or molecular chaperone itself, to the prokaryotic cell-free transcription/translation environment (e.g., such as the reduced acellular environment described herein), during the expressing the plurality of DNA constructs of the CIS display library.

In order to allow cis activity, a coupled bacterial transcription/translation environment such as the S30 extract system (see e.g., Zubay, G. 1973. Ann. Rev. Genet. 7:267) may be used. Expression of the DRP-chaperone-replication initiator protein or DRP-replication initiator protein, such as the DNA library member DRP-chaperone-RepA fusion protein or DNA library member DRP-RepA fusion protein, in this environment, will result in binding of the fusion protein to the DNA encoding that fusion protein, provided that both cis and ori sequences are present. When libraries of DRP fusion proteins are expressed in this manner, this process results in the production of libraries of protein-DNA complexes where the protein attached to the DNA is encoded by that fragment of DNA from which it was expressed, thereby allowing subsequent selection of both peptides or protein of interest, and the DNA encoding said peptides. The complexity of these libraries is enhanced by the in vitro nature of the method, libraries of at least 10⁴-10¹⁴, 10⁶-10¹⁴, or about 10¹⁰-10¹⁴ DNA fragments, if not even larger libraries, can easily be generated.

In some of any of the embodiments, the in vitro translation of individual or libraries of DRP-chaperone-RepA constructs or DRP-RepA constructs to express proteins is performed in a cell-free extract. Commonly used cell-free systems comprise extracts from Escherichia coli. All are prepared as crude extracts containing all the macromolecular components (70S or 80S ribosomes, tRNAs, aminoacyl-tRNA synthetases, initiation, elongation and termination factors, etc.) required for translation of exogenous RNA. To ensure efficient translation, each extract must be supplemented with amino acids, energy sources (ATP, GTP), energy regenerating systems (creatine phosphate and creatine phosphokinase for eukaryotic systems, and phosphoenolpyruvate and pyruvate kinase for the E. coli lysate), and other co-factors (Mg2+, K+, etc.). These systems are considered to be “coupled” and “linked” systems, as they start with DNA templates, which are transcribed into RNA then translated.

In some embodiments, an E. coli cell-free system is used to express a CIS display library in a reduced acellular environment. In some embodiments, the E. coli cell-free system is a “linked” or “coupled” system, which uses DNA as a template. RNA is transcribed from the DNA and subsequently translated without any purification. Such systems typically combine a prokaryotic phage RNA polymerase and promoter (T7, T3, or SP6) with eukaryotic or prokaryotic extracts to synthesize proteins from exogenous DNA templates. DNA templates for transcription: translation reactions may be cloned into plasmid vectors or generated by PCR.

In vitro E. coli transcription-translation reactions are performed in the same tube. During transcription, the 5′ end of the RNA becomes available for ribosomal binding and undergoes translation while its 3′ end is still being transcribed. This early binding of ribosomes to the RNA maintains transcript stability and promotes efficient translation. This bacterial translation system gives efficient expression of either prokaryotic or eukaryotic gene products in a short amount of time. In some embodiments, the DNA template has a Shine-Dalgarno ribosome binding site upstream of the initiator codon for higher protein yield and the higher initiation fidelity. Also, the E. coli S30 extract system allows expression from DNA vectors containing natural E. coli promoter sequences (such as lac or tac).

Compounds that prevent nuclease activity or reduce non-specific DNA-protein or protein-protein interactions may be added during this transcription/translation reaction and cis-binding. Examples of suitable compounds include detergents and blocking proteins such as bovine serum albumin (BSA).

The pairing of disulfide bonds and correct folding of expressed DRPs occurs in a reducing environment when a coupled bacterial transcription/translation reaction is performed to express the DRP-chaperone-RepA fusion protein or DRP-RepA fusion protein. A reducing environment prevents the correct formation of disulfide bonds, resulting in instability and aggregation of DRPs in said environment. The presence of one or more molecular chaperones, provided by expression from a DNA sequence encoding the molecular chaperone included in each of the DNA constructs or added to the prokaryotic cell-free transcription/translation environment, during the expressing the plurality of DNA constructs, allows the correct formation of disulfide bonds and subsequently increases stability and functionality of DRPs, including in a reducing environment. The methods provided herein represent a significant improvement over the standard CIS Display method as, surprisingly, it allows for proper folding and display of disulfide rich proteins (e.g., 2 or more disulfide bonds) with chaperone-RepA fusions, or RepA fusions in the presence of a separately encoded chaperone, and subsequent selection of binding DRPs from screening DRP libraries with targets of interest (e.g., ligands).

Provided in methods herein, the fusion protein (e.g., DRP-chaperone-RepA or DRP-RepA) is transcribed and translated from the DNA construct in an in vitro transcription-translation reaction. In some embodiments, RepA or a fragment or variant thereof capable of DNA binding, including at least the 20 C-terminal amino acids of RepA capable of binding to the cis DNA element is translated before the cis element. As the cis element is required for cis activity of the RepA protein, the cis DNA element should therefore also be located 3′ in the DNA construct to the DNA encoding the RepA sequence. (See e.g., Praszkier and Pittard 1999 J. Bacteriol. 181:2765-2772). On reaching the cis sequence, the RNA polymerase will be paused, allowing the encoded RepA protein to bind the DNA target sequence. In some embodiments, the DNA target sequence comprises an ori sequence, for example the oriR sequence, which binds RepA. In some of any of the embodiments, the DNA-protein binding is direct in that the peptide (e.g., RepA) encoded by the DNA construct will bind directly to the encoding DNA construct. The fusion protein is then transcribed and translated from the DNA construct and the final product, DNA:protein complex, can be further utilized in downstream applications.

### D. Methods of Screening and Target Selection

Also provided herein are methods for selecting, from any of the CIS display libraries described herein, a binding disulfide rich protein that is specific for a target molecule. The protein-DNA complexes of interest may be selected from a library by, for example, affinity or activity enrichment techniques to identify the DRP of interest. For example, protein-DNA complexes may be isolated by capture of a target protein and unbound protein-DNA complexes may be washed away, allowing enrichment for DNA encoding DRPs of interest, which can then be recovered by PCR, and enriched further by performing several further cycles of in vitro expression and protein-DNA complex capture using methods described below.

In some aspects, the CIS display libraries are contacted with a target of interest (e.g., a target molecule) and those members of the library having the highest affinity for the target are separated from those with lower affinity. The binders with improved affinity are then amplified by any suitable system. This process is reiterated until polypeptides of the desired affinity are obtained.

In some aspects, an in vitro CIS Display library produced by a method of the present invention may be used to screen for particular DRP members of the library. This can be accomplished by means of a ligand specific for the protein of interest, such as an antigen if the protein of interest is an antibody. The ligand may be presented on a solid surface such as the surface of an ELISA plate well, or in solution, for example, with biotinylated ligand followed by capture onto a streptavidin coated surface or magnetic beads, after a library of protein-DNA complexes had been incubated with the ligand to allow ligand-ligand interaction. Following either solid phase or in solution incubation, unbound complexes are removed by washing, and bound complexes isolated by disrupting ligand-ligand interactions by altering pH in the well, or by other methods known to those skilled in the art such as protease digestion, or by releasing the DNA directly from the complexes by heating or phenol-chloroform extraction to denature the RepA-ori DNA binding. DNA can also be released by one of the methods above, directly into PCR buffer, and amplified. Alternatively, DNA may be PCR amplified directly without release from the complexes. Optionally, DNA not bound by the binding for example RepA protein, can be protected from degradation by non-specific DNA binding proteins such as histones, by way of example. It will be clear to one skilled in the art that many other non-specific DNA binding proteins could be used for this purpose. Further, compounds that prevent nuclease activity, or reduce non-specific DNA-protein or protein-protein interactions may be present during the selection process. Examples of suitable compounds include detergents, blocking proteins such as found in milk powder or bovine serum albumin (BSA), heparin or aurintricarboxylic acid.

Recovering bound complexes, reamplifying the bound DNA, and repeating the selection procedure provides an enrichment of clones encoding the desired sequences, which may then be isolated for sequencing, further cloning and/or expression. For example, the DNA encoding the DRP of interest may be isolated and amplified by, for example PCR. In one embodiment, repeated rounds of selection and DNA recovery may be facilitated by the use of sequential nesting of PCR primers. DNA ends are generally damaged after multiple PCR steps. To recover DNA from such damaged molecules required the primers to be annealed away from the ends of the DNA construct, thereby sequentially shortening the construct with every round of selection.

In one aspect, the DNA construct and/or the encoded protein may be configured to include a tag. Such a peptide or DNA tag, for example as described above, may be used in the separation and isolation of a library member of interest. Such a tag may also be used to hold the library members, for example on a solid support for use in the screening methods described herein.

It can therefore be seen that the screening methods of the present invention may include the further step of selecting and isolating the relevant library member DRP, allowing the DRP exhibiting the desired properties, and also the DNA encoding that DRP, to be identified and purified.

The invention therefore encompasses peptides (e.g., DRPs, peptide tag) and DNAs that have been identified by a method of the invention. These peptides and DNAs may be isolated and/or purified. The peptides or DNAs isolated by a method of the invention may be modified, for example by deletion, addition or substitution of amino acids or nucleotides. Suitable modified peptides or DNAs may show at least 50%, at least 75%, at least 90%, at least 95% or more amino acid or nucleotide sequence identity to the peptide or DNA isolated by the method of the invention. Peptides identified by a method of the invention may be modified for delivery and/or stability purposes. For example, such peptides may be pegylated (attached to polyethylene glycol) to prolong serum half-life or to prevent protease attack. Peptides identified by a method of the invention may be modified in other display systems such as phage display or by synthesizing and screening peptide variants. A collection of such modified sequences may form a new library which may be incorporated into constructs of the invention and further screened to find, for example, a variant sequence showing improved binding to a particular ligand. Thus, in one embodiment, a library of peptides for use in the methods of the invention may be a library of structurally related peptides.

For instance, the CIS display library is a CIS DRP display library as described herein in which the DRP library members comprise CDR3-knob peptides. Once expressed, the protein: DNA fusions are then contacted with a target molecule and those complexes having the highest affinity for the target are separated from those with lower affinity. The high affinity binders are then amplified by repeated screening and the competitive binding step is repeated. This process is reiterated until polypeptides of the desired affinity are obtained.

In some embodiments, the provided methods include contacting the CIS display libraries provided herein with a target molecule under conditions to allow binding of a display complex to the target molecule. In some embodiments, the methods further include separating the complexes that bind from those that do not, thereby selecting DRP complexes that include a DRP that binds to the target molecule. In some embodiments, the methods include sequencing the fusion gene in the selected complexes to identify the binding DRP.

Target molecules may be isolated from natural sources or prepared by recombinant methods by procedures known in the art. The purified target molecule can be attached to a suitable matrix such as agarose beads, acrylamide beads, glass beads, cellulose, various acrylic copolymers, hydroxyalkyl methacrylate gels, polyacrylic and polymethacrylic copolymers, nylon, neutral and ionic carriers, and the like. Attachment of the target protein to the matrix may be accomplished by methods described in Methods in Enzymology, 44 1976, or by other means known in the art.

After attachment of the target molecule to the matrix, the immobilized target can be contacted with the library of DRP DNA:protein fusion under conditions suitable for binding of at least a portion of the display particles with the immobilized target molecules. Normally, the conditions, including pH, ionic strength, temperature and the like will mimic physiological conditions. Exemplary “contacting” conditions may comprise incubation for 15 minutes to 4 hours, e.g., one hour, at 4°-37° C., e.g., at room temperature. However, these may be varied as appropriate depending on the nature of the interacting binding partners, etc. The mixture can be subjected to gentle rocking, mixing, or rotation. In addition, other appropriate reagents such as blocking agents to reduce nonspecific binding may be added. For example, 1-4% BSA or other suitable blocking agent (e.g., milk) may be used. It will be appreciated however that the contacting conditions can be varied and adapted by a skilled person depending on the aim of the screening method. For example, if the incubation temperature is, for example, room temperature or 37° C., this may increase the possibility of identifying binders which are stable under these conditions, e.g., in the case of incubation at 37° C., are stable under conditions found in the human body. Such a property might be extremely advantageous if one or both of the binding partners was a candidate to be used in some sort of therapeutic application, e.g., an antibody. Again, such adaptations to the conditions are within the ambit of the skilled person.

Bound CIS display DRPs (“binders”) having high affinity for the immobilized target molecule can be separated from those having a low affinity (and thus do not bind to the target) by washing. Binders can be dissociated from the immobilized target molecules by a variety of methods. These methods include competitive dissociation using the wild-type ligand, altering pH and/or ionic strength, and methods known in the art.

In some embodiments, the target molecule is a nonvirulent bacterium, a virus, a viral protein, a cancer antigen, a human IgG, or a recombinant protein thereof. In some embodiments, the target molecule is a viral protein. In some embodiments, the target molecule is a coronavirus, a coronavirus pseudovirus, a recombinant coronavirus Spike protein, or a receptor-binding domain (RBD) of a coronavirus Spike protein. In some embodiments, the coronavirus is selected from the group consisting of 229E, NL63, OC43, HKU1, MERS-CoV, SARS-CoV and SARS-CoV2. In some embodiments, the coronavirus is a SARS-CoV2 selected from Wuhan-Hu-1 isolate, B.1.351 South African variant or B.1.1.7 UK variant.

In some embodiments, the methods include steps wherein previously selected DRPs are re-expressed and subjected to further selection steps, including with the same or a different target molecule. In some embodiments, the selection steps are repeated one or more times. In some embodiments, the further selection steps include repeating the in vitro transcription-translation (ITT) reaction with the replicable expression vectors encoding the previously selected DRPs; collecting additional expressed DRPs; and contacting the additional expressed DRPs with the same or a different target antigen. In some embodiments, the different target molecule is related to the target molecule and is the same type of pathogen, the same group of pathogens, or a variant of the target molecule. In some embodiments, the target molecule and different target molecule are associated with any combination of coronaviruses 229E, NL63, OC43, HKU1, MERS-CoV, SARS-CoV, and SARS-CoV2. In some embodiments, the target molecule and different target molecule are associated with any combination of SARS-CoV2 variants selected from Wuhan-Hu-1 isolate, B.1.351 South African variant, and B.1.1.7 UK variant.

Once one or more sets of binders have been selected or isolated in accordance with the provided methods, these can be subjected to further analysis. In some embodiments, the further analysis involves the isolation of binders by infection of bacteria as an amplification step, isolating the DNA associated with the binder (e.g., DRP), and cloning the DNA sequence encoding the candidate binders contained in said DRP DNA into a suitable expression vector. Such a step can also allow the amplification of the binders. Alternatively, binders can be amplified at this stage by other appropriate methods, for example by PCR of the nucleic acids encoding said binders or the transformation of said nucleic acid into an appropriate host cell (in the context of a suitable expression vector).

Once the DNA encoding the binders are cloned in a suitable expression vector, the DNA encoding the binders can be sequenced or the protein can be expressed in a soluble form, e.g., including according to the methods provided herein, and subjected to appropriate binding studies to further characterize the candidates at the protein level. Appropriate binding studies will depend on the nature of the binders, and include, but are not limited to ELISA, filter screening assays, FACS, or immunofluorescence assays, BiaCore affinity measurements or other methods to quantify binding constants, staining tissue slides or cells and other immunohistochemistry methods. One or more of these binding studies can be used to analyze the binders. Once the DNA encoding the binders are cloned in a suitable expression vector, the DNA encoding the binders can be sequenced or the protein can be expressed in a soluble form, e.g., including according to the methods provided herein, and subjected to appropriate binding studies to further characterize the candidates at the protein level.

### E. Expressing Disulfide Rich Proteins of Interest

After methods of the present invention are used to screen for particular disulfide rich protein (DRP) members of the library, provided herein in some embodiments are methods of producing said soluble DRP, including methods of producing any of the DRPs (also referred to as binders) identified by any of the methods described herein. The DRPs identified after screening with a target of interest (e.g., refer to Section II.D) may be expressed by cloning the DNA encoding the selected library member into a vector. In some embodiments, the identified candidate DRP has improved binding to a particular ligand. In some embodiments, producing said candidate DRP using the methods provided herein results in improved expression levels and solubility. In some embodiments, the one or more DRP of interest was identified as a screen hit. In some embodiments, the DRP identified as a screen hit has a desired size of effect (e.g., improved binding affinity).

The soluble DRPs (e.g., soluble peptides) produced by the provided methods are peptides (e.g., of 25 to 70 amino acids in length) that contain 4 or more cysteine residues from which it is desired to produce a disulfide-bonded soluble protein. In some embodiments, the provided methods include transforming a host cell, e.g., E. coli, with an expression vector encoding the soluble peptide. In some embodiments, the expression vector encodes a fusion protein that includes the soluble peptide and a chaperone, e.g., a bacterial chaperone. In some embodiments, the soluble peptide and the chaperone, e.g., bacterial chaperone, are joined by a linker. In some embodiments, the linker is a cleavable linker.

In some embodiments, the fusion protein has increased solubility relative to the soluble protein alone. In some aspects, this increased solubility is conferred at least in part by the inclusion of the chaperone, e.g., bacterial chaperone. In some aspects, the inclusion of the chaperone, e.g., bacterial chaperone, promotes solubility of the fusion protein while permitting disulfide bond formation in the soluble peptide, including in host cell environments that have been engineered or modified to promote disulfide bond formation. In some embodiments, the chaperone, e.g., bacterial chaperone, is thioredoxin A (TrxA).

In some embodiments, the soluble peptide is up to 300 amino acids in length. In some embodiments, the soluble peptide is 40 to 60 amino acids in length. In some embodiments, the soluble peptide is at least 42 amino acids in length. In some embodiments, the soluble peptide is 42 amino acids, 43 amino acids, 44 amino acids, 45 amino acids, 46 amino acids, 47 amino acids, 48 amino acids, 49 amino acids, 50 amino acids, 51 amino acids, 52 amino acids, 53 amino acids, 54 amino acids, 55 amino acids, 56 amino acids, 57 amino acids, 58 amino acids, 59 amino acids or 60 amino acids in length. In some of any embodiments, the DRP is at least 65 amino acids, 70 amino acids, 75 amino acids, 80 amino acids, 85 amino acids, 90 amino acids, 95 amino acids, 100 amino acids, 105 amino acids, 110 amino acids, 120 amino acids, 130 amino acids, 140 amino acids, 150 amino acids, 160 amino acids, 170 amino acids, 180 amino acids, 190 amino acids, 200 amino acids, 225 amino acids, 250 amino acids, 275 amino acids, or at least 300 amino acids in length.

In some embodiments, the soluble peptide is 25-300 amino acids. For instance, in some embodiments the soluble peptide is 35 amino acids in length or longer, 40 amino acids in length or longer, 45 amino acids in length or longer, 50 amino acids in length or longer, 55 amino acids in length or longer, or 60 amino acids in length or longer. In some embodiments, the soluble peptide is between or between about 35 and 70 amino acids in length, 40 and 70 amino acids in length, 45 and 70 amino acids in length, 50 and 70 amino acids in length, 55 and 70 amino acids in length, or 60 and 70 amino acids in length.

In some embodiments, the soluble peptide is 6 to 50 amino acids, 6 to 40 amino acids, 6 to 30 amino acids, 6 to 25 amino acids, 6 to 20 amino acids, 6 to 15 amino acids, 6 to 10 amino acids, 10 to 50 amino acids, 10 to 40 amino acids, 10 to 30 amino acids, 10 to 25 amino acids, 10 to 15 amino acids, 15 to 50 amino acids, 15 to 40 amino acids, 15 to 30 amino acids, 15 to 25 amino acids, 15 to 20 amino acids, 20 to 50 amino acids, 20 to 40 amino acids, 20 to 30 amino acids, 20 to 25 amino acids, 25 to 50 amino acids, 25 to 40 amino acids, 25 to 30 amino acids, 30 to 50 amino acids, 30 to 40 amino acids, or 40 to 50 amino acids. In some embodiments, the soluble peptide is 6 to 30 amino acids, 6 to 24 amino acids, 6 to 18 amino acids, 6 to 12 amino acids, 12 to 30 amino acids, 12 to 24 amino acids, 12 to 18 amino acids, 18 to 30 amino acids, 18 to 24 amino acids or 24 to 30 amino acids. In some embodiments, the soluble peptide is 55 to 60 amino acids, 60 to 80 amino acids, 80 to 100 amino acids, 100 to 125 amino acids, 125 to 150 amino acids, 150 to 175 amino acids, 175 to 200 amino acids, 200 to 225 amino acids, 225 to 250 amino acids, 250 to 275 amino acids, or 275 to 300 amino acids.

In some embodiments, the soluble peptide includes a cysteine motif able to form disulfide bonds. In some embodiments, the cysteine motif includes 2-20 cysteine residues, for instance between or between about 2 and 18, 2 and 16, 2 and 14, 2 and 12, 2 and 10, 2 and 8, 2 and 6, 2 and 4, 4 and 20, 4 and 18, 4 and 16, 4 and 14, 4 and 12, 4 and 10, 4 and 8, 4 and 6, 6 and 20, 6 and 18, 6 and 16, 6 and 14, 6 and 12, 6 and 10, 6 and 8, 8 and 20, 8 and 18, 8 and 16, 8 and 14, 8 and 12, 8 and 10, 10 and 20, 10 and 18, 10 and 16, 10 and 14, 10 and 12, 12 and 20, 12 and 18, 12 and 16, 12 and 14, 14 and 20, 14 and 18, 14 and 16, 16 and 20, 16 and 18, or 18 and 20 cysteine residues, each inclusive. In some embodiments, the cysteine motif includes 2-12 cysteine residues. In some embodiments, the soluble peptide comprises at least 4 Cys residues. In some embodiments, the soluble peptide contains 4 Cys residues. In some embodiments, the soluble peptide contains 6, 8, 10, or 12 Cys residues.

In some embodiments, the soluble peptide includes 2-10 disulfide bonds, for instance between or between about 2 and 9, 2 and 8, 2 and 7, 2 and 6, 2 and 5, 2 and 4, 2 and 3, 2 and 10, 2 and 9, 2 and 8, 2 and 7, 2 and 6, 2 and 5, 2 and 4, 2 and 3, 3 and 10, 3 and 9, 3 and 8, 3 and 7, 3 and 6, 3 and 5, 3 and 4, 4 and 10, 4 and 9, 4 and 8, 4 and 7, 4 and 6, 4 and 5, 5 and 10, 5 and 9, 5 and 8, 5 and 7, 5 and 6, 6 and 10, 6 and 9, 6 and 8, 6 and 7, 7 and 10, 7 and 9, 7 and 8, 8 and 10, 8 and 9, or 9 and 10 disulfide bonds, each inclusive. In some embodiments, the soluble peptide includes 2-6 disulfide bonds. In some embodiments, the soluble peptide contains 2-4 disulfide bonds. In some embodiments, the soluble peptide has at least 2 disulfide bonds. In some embodiments, the soluble peptide has 2 disulfide bonds. In some embodiments, the soluble peptide has 3, 4, or 5 disulfide bonds.

In some embodiments, the soluble peptide includes 3-6 amino acids preceding the most N-terminal cysteine residue present in the soluble peptide. In some embodiments, the soluble peptide includes 3, 4, 5, or 6 amino acids preceding the most N-terminal cysteine residue present in the soluble peptide.

In some embodiments, the soluble peptide includes at least 6 amino acids following the most C-terminal cysteine residue present in the soluble peptide. In some embodiments, the soluble peptide includes 6-9 amino acids following the most C-terminal cysteine residue present in the soluble peptide. In some embodiments, the soluble peptide includes 6, 7, 8, or 9 amino acids following the most C-terminal cysteine residue present in the soluble peptide.

In some embodiments, the soluble peptide includes a flexible linker. In some embodiments, the flexible linker is included at the N-terminus of the soluble peptide. In some embodiments, the flexible linker is in addition to the 3-6 amino acids preceding the most N-terminal cysteine residue present in the soluble peptide. In some embodiments, the flexible linker is included in the 3-6 amino acids preceding the most N-terminal cysteine residue present in the soluble peptide. In some embodiments, the flexible linker is included at the C-terminus of the soluble peptide. In some embodiments, the flexible linker is in addition to the at least 6 amino acids following the most C-terminal cysteine residue present in the soluble peptide. In some embodiments, the flexible linker is included in the at least 6 amino acids following the most C-terminal cysteine residue present in the soluble peptide.

In some embodiments, the flexible linker allows for cyclization of the soluble peptide. In some embodiments, the cyclization is via chemical or enzymatic methods. In some embodiments, the flexible linker allows for sortase-mediated cyclization of the soluble peptide. In some embodiments, the provided methods further include a step of cyclizing the soluble peptide, e.g., via chemical or enzymatic methods.

In some embodiments, the provided methods further include steps for enriching for the soluble peptide. In some embodiments, the provided methods further include separating the soluble peptide from any soluble aggregates present in solution, including soluble aggregates of the soluble peptide. In some embodiments, the separating involves the active soluble peptide from the larger, inactive, or less active soluble aggregates thereof. In some embodiments, the separating is achieved using chromatographic methods. In some embodiments, the enriching or separating is by size exclusion chromatography. In some embodiments, the separating involves collecting one or more elution fractions containing the soluble peptide, but not the soluble aggregates thereof, thereby producing an enriched or purified composition of soluble peptides.

In some embodiments, the provided methods further include producing a multispecific binding molecule that includes the soluble peptide. In some embodiments, the multispecific binding molecule includes multiple copies of the soluble peptide. In some embodiments, the multispecific binding molecule includes different soluble peptides. In some embodiments, the multispecific binding molecule includes a flexible linker (e.g., Gly-Gly-Gly-Ser) between the soluble peptides (e.g., between the C-terminus of one soluble peptide copy and the N-Terminus of the other soluble peptide copy). In some embodiments, one soluble peptide is present in a VH region that is expressed with a light chain as an IgG, and the second soluble peptide is fused to the heavy chain constant region. In some embodiments, the multispecific binding molecule includes two VH regions with the same soluble peptide. In some embodiments, the multispecific binding molecule includes VH regions that include different soluble peptides, for instance using heavy chains with constant region mutations such that only the heterologous heavy chains effectively pair with one another to form a dimer. In some embodiments, these mutations are ‘knobs-into-holes’ mutations, such as T22Y on one chain and Y86T on the other chain in the CH3 domain of Fc.

In some embodiments, the expression vector further includes an inducible promoter sequence to control the expression of the fusion protein. The term “promoter sequence” as used herein refers to a DNA sequence, which is generally located upstream of a gene present in a DNA polymer and provides a site for initiation of the transcription of said gene into mRNA. Promoter sequences suitable for use in this invention may be derived from viruses, bacteriophages, prokaryotic cells or eukaryotic cells, and may be a constitutive promoter or an inducible promoter.

In some embodiments, the inducible promoter sequence is operably linked to the sequence encoding the fusion protein. The term “operatively linked” as used herein means that a first sequence is disposed sufficiently close to a second sequence such that the first sequence can influence the second sequence or regions under the control of the second sequence. For instance, a promoter sequence may be operatively linked to a gene sequence and is normally located at the 5′-terminus of the gene sequence such that the expression of the gene sequence is under the control of the promoter sequence. In addition, a regulatory sequence may be operatively linked to a promoter sequence so as to enhance the ability of the promoter sequence in promoting transcription. In such case, the regulatory sequence is generally located at the 5′-terminus of the promoter sequence.

Promoter sequences suitable for use in this invention are preferably derived from any one of the following: viruses, bacterial cells, yeast cells, fungal cells, algal cells, plant cells, insect cells, animal cells, and human cells. For example, a promoter useful in bacterial cells includes, but is not limited to, tac promoter, T7 promoter, T7 A1 promoter, lac promoter, trp promoter, trc promoter, araBAD promoter, and λPRPL promoter. A promoter useful in plant cells includes, e.g., 35S CaMV promoter, actin promoter, ubiquitin promoter, etc. Regulatory elements suitable for use in mammalian cells include CMV-HSV thymidine kinase promoters, SV40, RSV-promoters, CMV enhancers, or SV40 enhancers.

Vectors suitable for use in this invention include those commonly used in genetic engineering technology, such as bacteriophages, plasmids, cosmids, viruses, or retroviruses.

Vectors suitable for use in this invention may include other expression control elements, such as a transcription starting site, a transcription termination site, a ribosome binding site, an RNA splicing site, a polyadenylation site, a translation termination site, etc. Vectors suitable for use in this invention may further include additional regulatory elements, such as transcription/translation enhancer sequences, and at least a marker gene or reporter gene allowing for the screening of the vectors under suitable conditions. Marker genes suitable for use in this invention include, for instance, dihydrofolate reductase gene and G418 or neomycin resistance gene useful in eukaryotic cell cultures, and ampicillin, streptomycin, tetracycline, or kanamycin resistance gene useful in E. coli and other bacterial cultures. Vectors suitable for use in this invention may further include a nucleic acid sequence encoding a secretion signal. These sequences are well known to those skilled in the art.

In some embodiments, the vector is a replicable expression vector that is a plasmid vector that generally contains a variety of components, including promoters, signal sequences, phenotypic selection genes, origin of replication sites, and other necessary components as are known to those of ordinary skill in the art. Promoters most commonly used in prokaryotic vectors include the lac Z promoter system, the alkaline phosphatase pho A promoter, the bacteriophage λPL promoter (a temperature sensitive promoter), the tac promoter (a hybrid trp-lac promoter that is regulated by the lac repressor), the tryptophan promoter, the bacteriophage T7 promoter, or other suitable microbial promoters. Examples of promoter systems include Lac Z, λPL, TAC, T 7 polymerase, tryptophan, and alkaline phosphatase promoters and combinations thereof. Suitable prokaryotic signal sequences may be obtained from genes encoding, for example, LamB or OmpF (Wong et al., Gene, 68:193 1983), MalE, PhoA, the E. coli heat-stable enterotoxin II (STII) signal sequence, or a Pel B secretory signal sequence. In some embodiments, the expression vector will further contain a secretory signal sequence operably fused to the nucleic acid encoding the polypeptide. In some embodiments, the secretory sequence is a Pel B secretory signal sequence. In some embodiments, the replicable expression vector also may contain a phenotypic selection gene. Typical phenotypic selection genes are those encoding proteins that confer antibiotic resistance upon the host cell. By way of illustration, the ampicillin resistance gene (amp), the tetracycline resistance gene (tet), or carbenicillin resistance gene may be used.

Construction of suitable vectors containing the nucleic acid encoding the desired polypeptide are prepared using standard recombinant DNA procedures. Isolated DNA fragments to be combined to form the vector are cleaved, tailored, and ligated together in a specific order and orientation to generate the desired vector. In some embodiments, the DNA is cleaved using the appropriate restriction enzyme or enzymes in a suitable buffer. Appropriate buffers, DNA concentrations, and incubation times and temperatures are specified by the manufacturers of the restriction enzymes. Generally, incubation times of about one or two hours at 37° C. are adequate, although several enzymes require higher temperatures. After incubation, the enzymes and other contaminants are removed by extraction of the digestion solution with a mixture of phenol and chloroform, and the DNA is recovered from the aqueous fraction by precipitation with ethanol.

To ligate the DNA fragments together to form a functional vector, the ends of the DNA fragments must be compatible with each other. In some cases, the ends will be directly compatible after endonuclease digestion. However, it may be necessary to first convert the sticky ends commonly produced by endonuclease digestion to blunt ends to make them compatible for ligation. To blunt the ends, the DNA is treated in a suitable buffer for at least 15 minutes at 15° C. with 10 units of the Klenow fragment of DNA polymerase I (Klenow) in the presence of the four deoxynucleotide triphosphates. The DNA is then purified by phenol-chloroform extraction and ethanol precipitation.

The DNA fragments that are to be ligated together (previously digested with the appropriate restriction enzymes such that the ends of each fragment to be ligated are compatible) are put in solution. In some embodiments, the DNA fragments are provided in about equimolar amounts. In some embodiments, the solution will also contain ATP, ligase buffer, and a ligase such as T4 DNA ligase, such as at or about 10 units per 0.5 μg of DNA. If the DNA fragment is to be ligated into a vector, the vector is first linearized by cutting with the appropriate restriction endonuclease(s). The linearized vector is then treated with alkaline phosphatase or calf intestinal phosphatase. The use of phosphatase prevents self-ligation of the vector during the ligation step.

In some embodiments, a plurality of constructed replicable expression vectors are transformed into suitable host cells. Suitable host cells include prokaryotes host cells. In some embodiments, the host cell used for expressing or producing the display libraries are E. coli cells. Suitable prokaryotic host cells include E. coli strain JM101, E. coli K12 strain 294 (ATCC number 31,446), E. coli strain W3110 (ATCC number 27,325), E. coli X1776 (ATCC number 31,537), E. coli XL-1Blue (stratagene), and E. coli B; however, many other strains of E. coli, such as HB101, NM522, NM538, NM539, and many other species and genera of prokaryotes may be used as well. In addition to the E. coli strains listed above, bacilli such as Bacillus subtilis, other Enterobacteriaceae such as Salmonella typhimurium or Serratia marcesans, and various Pseudomonas species may all be used as hosts. In some embodiments, the host cell is a protease deficient strain of E. coli. In some embodiments, the host cells are TG1 electrocompetent cells.

Transformation of prokaryotic cells is readily accomplished using the calcium chloride method as described in section 1.82 of Sambrook et al., supra. Alternatively, electroporation (Neumann et al., EMBO J., 1:841 1982) may be used to transform these cells. The transformed cells are selected by growth on an antibiotic, for example tetracycline (tet) or ampicillin (amp), carbenicillin or other antibiotic depending on the particular expression vector, to which they are rendered resistant due to the presence of resistance genes on the vector.

After selection of the transformed cells, these cells are grown in culture and the plasmid DNA (or other vector with the foreign gene inserted) is then isolated. Plasmid DNA can be isolated using methods known in the art. The isolated DNA can be purified by methods known in the art. This purified plasmid DNA is then analyzed by restriction mapping and/or DNA sequencing.

Depending on the vector and host cell system used, the recombinant gene product (protein) produced according to this invention may either remain within the recombinant cell, be secreted into the culture medium, be secreted into periplasm, or be retained on the outer surface of a cell membrane. The recombinant gene product (protein) produced by the method of this invention can be purified by using a variety of standard protein purification techniques, including, but not limited to, affinity chromatography, ion exchange chromatography, gel filtration, electrophoresis, reverse phase chromatography, chromatofocusing and the like. The recombinant gene product (protein) produced by the method of this invention is preferably recovered in “substantially pure” form. As used herein, the term “substantially pure” refers to a purity of a purified protein that allows for the effective use of said purified protein as a commercial product.

i. Host Cells

The term “host cell” is used to refer to a cell which has been transformed, transfected or infected or is capable of being transformed, transfected or infected with a nucleic acid sequence and then of expressing a selected gene of interest to recombinantly produce a protein of interest. The term includes the progeny of the parent cell, whether or not the progeny is identical in morphology or in genetic make-up to the original parent, so long as the selected gene or genetic modification is present.

The provided methods for producing a soluble peptide or a fusion protein containing the soluble peptide and optionally a chaperone, e.g., bacterial chaperone, can be performed using any host organism which is capable of expressing heterologous polypeptides, and is capable of being genetically modified. A host organism is preferably a unicellular host organism, however, the use of multicellular organisms is also encompassed by the provided methods, provided the organism can be modified as described herein and a polypeptide of interest expressed therein. For purposes of clarity, the term “host cell” will be used herein throughout, but it should be understood, that a host organism can be substituted for the host cell, unless unfeasible for technical reasons.

In some embodiments, the host cell is a prokaryotic cell, such as a bacterial cell. The host cell may be a gram-positive bacterial cell, such as Bacillus or gram negative bacteria such as E. coli. The host organisms may be aerobic or anaerobic organisms. In some embodiments, host cells are those which have characteristics which are favorable for expressing polypeptides, such as host cells having fewer proteases than other types of cells. Suitable bacteria for this purpose include archaebacteria and eubacteria, for example, Enterobacteriaceae. Other examples of useful bacteria include Escherichia, Enterobacter, Azotobacter, Erwinia, Bacillus, Pseudomonas, Klebsiella, Proteus, Salmonella, Serratia, Shigella, Rhizobia, Vitreoscilla, and Paracoccus. Additional examples of useful bacteria include Corynebacterium, Lactococcus, Lactobacillus, and Streptomyces species, in particular Corynebacterium glutamicum, Lactococcus lactis, Lactobacillus plantarum, Streptomyces coclicolor, Streptomyces lividans. Suitable E. coli hosts include E. coli DHB4, E. coli BL-21 (which are deficient in both Ion (Phillips et al. J. Bacteriol. 159:283, 1984) and ompT proteases), E. coli AD494, E. coli W3110 (ATCC 27,325), E. coli 294 (ATCC 31,446), E. coli B, and E. coli X1776 (ATCC 31,537). Other strains include E. coli B834 which are methionine deficient and, therefore, enables high specific activity labeling of target proteins with ³⁵S-methionine or selenomethionine (Leahy et al. Science 258:987, 1992). Yet other strains of interest include the BLR strain, and the K-12 strains HMS174 and NovaBlue, which are recA-derivative that improve plasmid monomer yields and may help stabilize target plasmids containing repetitive sequences.

In some embodiments, the E. coli host cell used in the provided methods is engineered or modified to improve soluble expression of disulfide-bonded proteins in the E. coli cytosol. In some embodiments, the cytoplasmic thiol-redox equilibrium environment is changed via alteration in reducing pathways, such as thioredoxin reductase. In some embodiments, the E. coli host cell has an oxidizing cytoplasm that is permissive of disulfide bond formation. Various types of mutant strains, including Shuffle (New England Biolabs) and Origami™ (DE3) (Novagen, Germany), which lack glutathione reductase Δgor, thioredoxin reductase, and/or glutathione biosynthesis pathways, are commercially available. In some embodiments, the E. coli strain transformed as part of the provided methods is the Origami™ (DE3) (Novagen, Germany) mutant strain.

Suitable Bacillus strains include Bacillus subtilis, Bacillus anzyloliguelaciens, Bacillus licheniformis, Bacillus brevis, Bacillus alcalophilus, Bacillus clauseii, Bacillus cereus, Bacillus pumilus, Bacillus thuringiensis, or Bacillus halodurans. The Gram-positive bacterium B. subtilis is a preferred organism for secretory protein production in the biotechnological industry. Its popularity is primarily based on the fact that B. subtilis lacks an outer membrane, which retains many proteins in the periplasm of Gram-negative bacteria such as Escherichia coli. Accordingly, the majority of B. subtilis proteins that are transported across the cytoplasmic membrane end up directly in the growth medium. Additionally, the lack of an outer membrane implies that proteins produced with B. subtilis are free from lipopolysaccharide (endotoxin). Other advantages of using B. subtilis as a protein production host are its high genetic amenability, the availability of strains with mutations in nearly all of the ^{˜}4100 genes, a toolbox with strains and vectors for gene expression, and the fact that this bacterium is generally recognized as safe (Braun et al., Curr. Opin. Biotechnol. 10:376-381, 1999; Kobayashi et al., Proc. Natl. Acad. Sci. U.S.A 100:4678-4683, 2003; Kunst et al. Nature 390:249-256, 1997; Zeigler et al., In E. Goldman and L. Green (ed.), Practical Handbook of Microbiology. CRC Press, Boca Raton, Fla., 2008).

In another embodiment, the host cell is a eukaryotic cell, such as a yeast cell or a mammalian cell. Examples of mammalian cells include, but are not limited to Chinese hamster ovary cells (CHO) (ATCC No. CCL61), CHO DHFR-cells (Urlaub et al., Proc. Natl. Acad. Sci. USA, 97:4216-4220 (1980)), human embryonic kidney (HEK) 293 or 293T cells (ATCC No. CRL1573), or 3T3 cells (ATCC No. CCL92). The selection of suitable mammalian host cells and methods for transformation, culture, amplification, screening and product production and purification are known in the art. Other suitable mammalian cell lines, are the monkey COS-1 (ATCC No. CRL1650) and COS-7 cell lines (ATCC No. CRL1651), and the CV-1 cell line (ATCC No. CCL70). Further exemplary mammalian host cells include primate cell lines and rodent cell lines, including transformed cell lines. Normal diploid cells, cell strains derived from in vitro culture of primary tissue, as well as primary explants, are also suitable. Candidate cells may be genotypically deficient in the selection gene, or may contain a dominantly acting selection gene. Other suitable mammalian cell lines include but are not limited to, mouse neuroblastoma N2A cells, HeLa, mouse L-929 cells, 3T3 lines derived from Swiss, Balb-c or NIH mice, BHK or HaK hamster cell lines, which are available from the ATCC. Each of these cell lines is known by and available to those skilled in the art of protein expression.

Many strains of yeast cells known to those skilled in the art are also available as host cells for the expression of the polypeptides described herein. Exemplary yeast cells include, for example, Saccharomyces cerevisiae, and Pichia pastoris. Fungi, such as Aspergillum, are also available as host cells for the expression of the polypeptides described herein.

Additionally, where desired, insect cell systems may be utilized in the provided methods. Such systems are described for example in Kitts et al., Biotechniques, 14:810-817 (1993); Lucklow, Curr. Opin. Biotechnol., 4:564-572 (1993); and Lucklow et al. (J. Virol., 67:4566-4579 (1993). Exemplary insect cells are Sf-9 and Hi5 (Invitrogen, Carlsbad, Calif.).

### III. Exemplary Embodiments

Among the provided embodiments are:

1. A method of producing a disulfide rich protein (DRPs) CIS display library, the method comprising expressing a plurality of DNA constructs in a prokaryotic cell-free transcription/translation environment in the presence of at least one molecular chaperone,
wherein the at least one molecular chaperone is provided by (i) expression from a DNA sequence encoding the molecular chaperone included in the plurality of DNA constructs, or (ii) addition of one or more DNA sequences encoding the molecular chaperone or the molecular chaperone itself to the transcription/translation environment,
wherein each DNA construct comprises:
i. a DNA sequence encoding one of a plurality of disulfide rich protein (DRP);
ii. a DNA sequence encoding a DNA replication initiator protein; and
iii. a DNA sequence comprising a target sequence for the DNA replication,
thereby producing a plurality of protein fusions comprising the DRP and the DNA replication initiator protein and, optionally the molecular chaperone, from the plurality of DNA constructs,
wherein the protein fusion binds, via the DNA replication initiator protein, to the target sequence in the DNA construct from which it was transcribed, thereby linking the protein fusion to the DNA construct to produce the DRP CIS display library comprising a plurality of DNA:protein fusions.

2. The method of embodiment 1, wherein the molecular chaperone is provided by expression from a DNA sequence encoding the molecular chaperone included in each of the DNA constructs, and wherein each of the plurality of protein fusions comprises a DRP, the molecular chaperone, and the DNA replication initiator protein.

3. A method of producing a disulfide rich protein (DRPs) CIS display library, the method comprising:
(a) providing a plurality of DNA constructs each DNA construct comprising:
   i. a DNA sequence encoding one of a plurality of disulfide rich proteins (DRPs);
   ii. one or more DNA sequences encoding a molecular chaperone;
   iii. a DNA sequence encoding a DNA replication initiator protein; and
   iv. a DNA sequence comprising a target sequence for the DNA replication initiator protein, wherein the DNA replication initiator protein can non-covalently bind to the target sequence; and
(b) expressing the plurality of DNA constructs of (a) in a prokaryotic cell-free transcription/translation environment, thereby producing a plurality of protein fusions comprising the DRP, the molecular chaperone and the DNA replication initiator protein from the plurality of DNA constructs, and
wherein the protein fusion binds, via the DNA replication initiator protein, to the target sequence in the DNA construct from which it was transcribed, thereby linking the protein fusion to the DNA construct to produce the DRP CIS display library comprising a plurality of DNA:protein fusions.

4. The method of embodiment 1, wherein the method comprises the addition of one or more DNA sequences encoding the molecular chaperone or the molecular chaperone itself to the transcription/translation environment, wherein each of the plurality of protein fusions comprises a DRP and the DNA replication initiator protein.

5. A method of producing a disulfide rich protein (DRPs) CIS display library, the method comprising:
(a) providing a plurality of DNA constructs each DNA construct comprising:
   i. a DNA sequence encoding one of a plurality of disulfide rich proteins (DRPs);
   ii. a DNA sequence encoding a DNA replication initiator protein; and
   iii. a DNA sequence comprising a target sequence for the DNA replication initiator protein, wherein the DNA replication initiator protein can non-covalently bind to the target sequence;
(b) expressing the plurality of DNA constructs of (a) in a prokaryotic cell-free transcription/translation environment, thereby producing a plurality of protein fusions comprising the DRP and the DNA replication initiator protein from the plurality of DNA constructs;
c) adding a molecular chaperone, or one or more DNA sequences encoding a molecular chaperone, to the prokaryotic cell-free transcription/translation environment, during the expressing the plurality of DNA constructs in (b);
wherein the protein fusion binds, via the DNA replication initiator protein, to the target sequence in the DNA construct from which it was transcribed, thereby linking the protein fusion to the DNA construct to produce the DRP CIS display library comprising a plurality of DNA:protein fusions.

6. The method of embodiment 1-5, wherein the plurality of DRPs each have four or more cysteine residues for forming two or more disulfide bonds.

7. The method of any of embodiments 1-6, wherein each of the plurality of DNA constructs comprise a DNA sequence encoding a different DRP.

8. The method of any one of embodiments 1-7, wherein the plurality of DRP each comprise a natural and/or synthetic DRP.

9. The method of any one of embodiments 1-8, wherein the plurality of DRPs comprise an antibody, an scFv fragment, a cyclotide, a knottin, a toxin, and a CDR3 knob domain from an ultralong CDR3.

10. The method of any one of embodiments 1-9, wherein the plurality of DRPs comprise a peptide of 20-70 amino acids in length having 2-12 cysteine residues capable of forming 1-6 intramolecular disulfide bonds.

11. The method of embodiment 10, wherein the peptide comprises 4-8 cysteine residues forming 2-4 intramolecular disulfide bonds.

12. The method of embodiment 10 or embodiment 11, wherein the peptide is a knob domain derived from an ultralong CDR3 of an antibody.

13. The method of embodiment 12, wherein the ultralong CDR3 of an antibody is from a member of the Bovinae subfamily.

14. The method of embodiment 12 or embodiment 13, wherein the ultralong CDR3 of an antibody is from a bovine, optionally Bos taurus.

15. The method of any one of embodiments 1-14, wherein the plurality of DRPs comprise DRP variants comprising one or more differences in their amino acid residues, optionally wherein the amino acid differences comprise substitutions, insertions, deletions, or combinations thereof.

16. The method of embodiment 15, wherein the amino acid differences are introduced by random mutagenesis of the DNA sequence encoding the DRP.

17. The method of embodiment 15, wherein the amino acid differences are introduced by incorporation of degenerate codons during oligonucleotide synthesis of the DRP-encoding DNA.

18. The method of any one of embodiments 15-17, wherein the amino acid differences preserve the cysteine motif and disulfide-bonds.

19. The method of any one of embodiments 1-18, wherein the plurality of DRPs comprise at least 10³, at least 10⁵, at least 10⁷, or at least 10⁹ unique members, each member comprising a distinct DRP sequence.

20. The method of any one of embodiments 1-19, wherein the one or more encoded molecular chaperones comprise a bacterial or eukaryotic origin.

21. The method of any one of embodiments 1-20, wherein the one or more encoded molecular chaperones comprise a molecular weight of 10 kD to 80 kD.

22. The method of any one of embodiments 1-21, wherein the one or more encoded molecular chaperones comprise a molecular weight of 10 kD to 24 kD.

23. The method of any one of embodiments 1-22, wherein the one or more encoded molecular chaperones comprise trxA, DsbA, DsbB, DsbC, HSP70, DnaK, HSP90, GroES, GroEL, DNAK, Sumo and Trigger Factor, or protein disulfide isomerase (PDI).

24. The method of any one of embodiments 1-23, wherein the one or more encoded molecular chaperones comprise trxA, DsbA, DsbB, DsbC, HSP70, and/or DnaK.

25. The method of any one of embodiments 1-24, wherein the one or more molecular chaperone is the molecular chaperone trxA.

26. The method of any one of embodiments 1-25, wherein the one or more molecular chaperone is the molecular chaperone DsbA.

27. The method of any one of embodiments 1-26, wherein each DRP of the CIS display library has formed two or more disulfide bonds.

28. The method of any one of embodiments 1-3 and 6-27, wherein the DNA sequence encoding the one or more encoded molecular chaperones is upstream and/or downstream of the DNA sequence encoding the DRP in the DNA construct.

29. The method of any one of embodiments 1-3 and 6-28, wherein the DNA sequence encoding the one or more molecular chaperones is upstream of the DNA sequence encoding the DRP in the DNA construct.

30. The method of any one of embodiments 1-3 and 6-29, wherein each of the plurality of DNA constructs comprises in order: the DNA sequence encoding the molecular chaperone, the DNA sequence encoding the DRP, the DNA sequence encoding the DNA replication initiator protein, and the DNA sequence encoding the target sequence for the DNA replication initiator protein.

31. The method of any one of embodiments 1-3 and 6-30, wherein each of the plurality of DNA constructs further comprises a DNA sequence encoding a first linker sequence between the molecular chaperone and the DRP and/or a DNA sequence encoding a second linker sequence between the DRP and the DNA replication initiator protein.

32. The method of embodiment 31, wherein the first linker is SEQ ID NO: 15.

33. The method of embodiment 31, wherein the second linker is SEQ ID NO: 15.

34. The method of any of embodiments 1-33, wherein the DNA replication initiator protein is RepA or a variant thereof that retains ability to bind to the target sequence.

35. The method of embodiment 34, wherein the target sequence for the DNA replication is a non-coding replication origin sequence recognized by the DNA replication initiator protein.

36. The method of any of embodiments 1-35, wherein the DNA replication initiator protein is RepA and the target sequence comprises an origin of replication sequence (ori).

37. The method of embodiment 36, wherein the DNA construct further comprises a cis-acting DNA element positioned between the DNA sequence encoding RepA and the target sequence comprising ori.

38. The method of embodiment 36 or embodiment 37, wherein the RepA is selected from the group consisting of RepA of the incompatibility group I (IncI) complex plasmids and RepA of the IncF, IncB, IncK, IncZ and IncL/M plasmids.

39 A polynucleotide comprising:
(a) a DNA sequence encoding a disulfide rich protein (DRP);
(b) a DNA sequence encoding one or more molecular chaperone;
(c) a DNA sequence encoding a DNA replication initiator protein; and
(d) a DNA sequence encoding a target sequence for the DNA replication initiator protein, wherein the DNA replication initiator protein can non-covalently bind to the target sequence.

40. A polynucleotide comprising:
(a) a DNA sequence encoding a disulfide rich protein (DRP);
(b) a DNA sequence encoding a DNA replication initiator protein; and
(c) a DNA sequence encoding a target sequence for the DNA replication initiator protein, wherein the DNA replication initiator protein can non-covalently bind to the target sequence.

41. The polynucleotide of embodiment 39 or embodiment 40, wherein the DRP has four or more cysteine residues for forming two or more disulfide bonds.

42. The polynucleotide of any one of embodiments 39-41, wherein the DNA sequence encoding the DRP comprises natural and/or synthetic DRPs.

43. The polynucleotide of any one of embodiments 39-42, wherein the DRP comprises an antibody, an scFv fragment, a cyclotide, a knottin, a toxin, and a CDR3 knob domain from an ultralong CDR3.

44. The polynucleotide of any one of embodiments 39-43, wherein the DRP comprises a peptide of 20-70 amino acids in length having 2-12 cysteine residues capable of forming 1-6 intramolecular disulfide bonds, optionally wherein the peptide comprises 4-8 cysteine residues forming 2-4 intramolecular disulfide bonds.

45. The polynucleotide of any one of embodiments 39-44, wherein the DRP is peptide that is a knob domain derived from an ultralong CDR3 of an antibody.

46. The polynucleotide of embodiment 45, wherein the ultralong CDR3 of an antibody is from a member of the Bovinae subfamily, optionally wherein the ultralong CDR3 of an antibody is from a bovine (e.g., Bos taurus).

47. The polynucleotide of any one of embodiments 39, and 41-46, wherein the DNA sequence encoding the one or more molecular chaperone nucleic acid sequences is upstream or downstream of the DNA sequence encoding the DRP in the polynucleotide.

48. The polynucleotide of any one of embodiments 39, and 41-47, wherein the DNA sequence encoding the one or more molecular chaperone is upstream of the DNA sequence encoding the DRP in the polynucleotide.

49. The polynucleotide of any one of embodiments 39, and 41-48, wherein the one or more molecular chaperone are a bacterial or eukaryotic origin.

50. The polynucleotide of any one of embodiments 39, and 41-49, wherein the one or more molecular chaperone have a molecular weight of 10 kD to 80 kD.

51. The polynucleotide of any one of embodiments 39, 41-50, wherein the one or more molecular chaperone have a molecular weight of 10 kD to 24 kD.

52. The polynucleotide of any one of embodiments 39, and 41-51, wherein the one or more molecular chaperone comprises trxA, DsbA, DsbB, DsbC, HSP70, DnaK, HSP90, GroES, GroEL, DNAK, Sumo and Trigger Factor, or protein disulfide isomerase (PDI).

53. The polynucleotide of any one of embodiments 39, and 41-52, wherein the one or more molecular chaperone are the molecular chaperone trxA.

54. The polynucleotide of any one of embodiments 38, 41-53 wherein the one or more molecular chaperone are the molecular chaperone DsbA.

55. The polynucleotide of any one of embodiments 39, and 41-54, wherein the polynucleotide further comprises a DNA sequence encoding a first linker sequence between the molecular chaperone and the DRP and/or a DNA sequence encoding a second linker sequence between the DRP and the DNA replication initiator protein.

56. The polynucleotide of embodiment 55, wherein the first linker is SEQ ID NO: 15 and/or the second linker is SEQ ID NO: 15.

57. The polynucleotide of any of embodiments 39-56, wherein the DNA replication initiator protein is RepA or a variant thereof that retains ability to bind to the target sequence.

58. The polynucleotide of embodiment 57, wherein the target sequence for the DNA replication is a non-coding replication origin sequence recognized by the DNA replication initiator protein.

59. The polynucleotide of any of embodiments 39-59, wherein the DNA replication initiator protein is RepA and the target sequence comprises an origin of replication sequence (ori).

60. The polynucleotide of embodiment 59, wherein the polynucleotide further comprises a cis-acting DNA element positioned between the nucleic acid sequence encoding RepA and the target sequence comprising ori.

61. The polynucleotide of embodiment 60, wherein the RepA is selected from the group consisting of RepA of the incompatibility group I (IncI) complex plasmids and RepA of the IncF, IncB, IncK, IncZ and IncL/M plasmids.

62. A composition, comprising the polynucleotide of any one of embodiments 39, and 41-61.

63. A composition, comprising the polynucleotide of embodiment 39-46 and 58-61.

64. The composition of embodiment 63, further comprising a molecular chaperone or a nucleic acid sequence encoding one or more molecular chaperone.

65. A DNA construct comprising the polynucleotide of embodiments 38-61.

66. An expression vector comprising the polynucleotide of embodiments 38-61 or the DNA construct of embodiment 65.

67. A DNA:protein fusion comprising:
(a) a protein fusion comprising:
   i. a disulfide rich protein (DRP);
   ii. one or more molecular chaperone proteins; and
   iii. a DNA replication initiator protein; and
(b) a DNA comprising:
   i. a DNA sequence encoding the DRP;
   ii. one or more DNA sequences encoding the molecular chaperone;
   iii. a DNA sequence encoding the DNA replication initiator protein; and
   iv. a DNA sequence comprising a target sequence for the DNA replication initiator protein;
wherein the DNA replication initiator protein non-covalently binds to the target sequence, thereby producing the DNA:protein fusion.

68. A DNA:protein fusion comprising:
(a) a protein fusion comprising:
   i. a disulfide rich protein (DRP); and
   ii. a DNA replication initiator protein; and
(b) a DNA comprising:
   i. a DNA sequence encoding the DRP;
   ii. a DNA sequence encoding the DNA replication initiator protein; and
   iii. a DNA sequence comprising a target sequence for the DNA replication initiator protein;
wherein the DNA replication initiator protein non-covalently binds to the target sequence, thereby producing the DNA:protein fusion.

69. The DNA:protein fusion of embodiment 67 or 68, wherein the DRP comprises a natural and/or synthetic DRP.

70. The DNA:protein fusion of any one of embodiments 67-69, wherein the DRP is an antibody, an scFv fragment, a cyclotide, a knottin, a toxin, and a cow CDR3 knob domain.

71. The DNA:protein fusion of any one of embodiments 67-70, wherein the DRP is a cysteine motif peptide of 20-50 amino acids in length having 2-12 cysteine residues capable of forming 1-6 intramolecular disulfide bonds.

72. The DNA:protein fusion of embodiment 71, wherein the cysteine motif peptide comprises 4-8 cysteine residues forming 2-4 intramolecular disulfide bonds.

73. The DNA:protein fusion of any one of embodiments 67-73, wherein the plurality of DRPs each are a cow CDR3 knob domain.

74. The DNA:protein fusion of embodiment 73, wherein the knob domain peptide is 40-70 amino acids in length and comprises at least four cysteine residues forming at least two intramolecular disulfide bonds.

75. The DNA:protein fusion of any one of embodiments 67, and 69-74, wherein the one or more molecular chaperones comprise a bacterial or eukaryotic origin.

76. The DNA:protein fusion of any one of embodiments 67, and 69-75, wherein the one or more molecular chaperones comprise a molecular weight of 10 kD to 80 kD.

77. The DNA:protein fusion of any one of embodiments 67, and 69-76, wherein the one or more molecular chaperones comprise a molecular weight of 10 kD to 24 kD.

78. The DNA:protein fusion of any one of embodiments 67, and 69-77, wherein the one or more molecular chaperones comprise trxA DsbA, DsbB, DsbC, HSP70, DnaK, HSP90, GroES, GroEL, DNAK, Sumo and Trigger Factor, or protein disulfide isomerase (PDI).

79. The DNA:protein fusion of any one of embodiments 67, and 69-78, wherein the one or more encoded molecular chaperones comprise trxA, DsbA, DsbB, DsbC, HSP70, and/or DnaK.

80. The DNA:protein fusion of any one of embodiments 67, and 69-79, wherein the one or more molecular chaperone is the molecular chaperone trxA.

81. The DNA:protein fusion of any one of embodiments 67, and 69-80, wherein the one or more molecular chaperone is the molecular chaperone DsbA.

82. The DNA:protein fusion of embodiments 67, and 69-81, wherein the one or more molecular chaperones are N-terminal or C-terminal of the DRP in the protein.

83. The DNA:protein fusion of any one of embodiments 67, and 69-82, wherein the one or more molecular chaperones are N-terminal of the DRP in the protein fusion.

84. The DNA:protein fusion of any one of embodiments 67, and 67-83, wherein the protein fusion further comprises a first linker sequence between the molecular chaperone and the DRP and/or a second linker sequence between the DRP and the DNA replication initiator protein.

85. The DNA:protein fusion of embodiment 84, wherein the first linker is SEQ ID NO: 15.

86. The DNA:protein fusion of embodiment 84, wherein the second linker is SEQ ID NO: 15.

87. The DNA:protein fusion of any one of embodiments 67-86, wherein the DRP protein has formed two or more disulfide bonds.

88. The DNA:protein fusion of any of embodiments 67-87, wherein the DNA replication initiator protein is RepA or a variant thereof that retains ability to bind to the target sequence.

89. The DNA:protein fusion of embodiment 88, wherein the target sequence for the DNA replication is a non-coding replication origin sequence recognized by the DNA replication initiator protein.

90. The DNA:protein fusion of any of embodiments 67-89, wherein the DNA replication initiator protein is RepA and the target sequence comprises an origin of replication sequence (ori).

91. The DNA:protein fusion of embodiment 90, wherein the RepA is selected from the group consisting of RepA of the incompatibility group I (IncI) complex plasmids and RepA of the IncF, IncB, IncK, IncZ and IncL/M plasmids.

92. A screening library produced according to the method of embodiments 1 to 37, comprising the plurality of DNA:protein fusion proteins.

93. A screening library comprising a plurality of DNA:protein fusion proteins of any of embodiments 67-91.

94. The screening library of embodiment 92 or embodiment 93 comprising 10⁶ to 10¹⁴ different DNA:protein fusions.

95. The screening library of embodiment 94 comprising 10¹⁰ to 10¹⁴ different DNA:protein fusions.

96. A method of screening a disulfide rich protein (DRP) CIS display library, the method comprising:
(a) providing a screening library comprising a plurality of DNA:protein fusion proteins according to embodiments 92-95;
(b) contacting members of the plurality of DNA:protein fusion proteins of the screening library with a target of interest to produce a mixture of the plurality of DNA:protein fusion proteins and the target of interest; and
(c) selecting from the mixture the DNA:protein fusion proteins that are bound to the target of interest.

97. The method of embodiment 96, further comprising identifying the one or more DNA:protein fusion proteins bound to the target of interest by identifying a sequence of the corresponding DNA:protein fusion replication initiator protein DNA.

98. The method of any one of embodiments 96-97, wherein contacting members of the plurality of DNA:protein fusion proteins with the target of interest is carried out in the presence of one or more molecular chaperones.

99. The method of any one of embodiments 96-98, wherein identifying the sequence of the corresponding DNA:protein fusion replication initiator protein DNA comprises nucleic acid sequencing.

Also, among the provided embodiments are:

1. A method of producing a disulfide rich protein (DRPs) CIS display library, the method comprising:
(a) providing a plurality of DNA constructs each DNA construct comprising:
   i. a DNA sequence encoding a DRP;
   ii. one or more DNA sequences encoding a molecular chaperone;
   iii. a DNA sequence encoding a DNA replication initiator protein; and
   iv. a DNA sequence encoding a target sequence for the DNA replication initiator protein, wherein the DNA replication initiator protein can non-covalently bind to the target sequence;
(b) expressing the plurality of DNA constructs of (a) in a prokaryotic cell-free transcription/translation environment, thereby producing a plurality of DRP-chaperone-DNA replication initiator proteins from the plurality of DNA constructs; and
(c) linking each of the DRP-chaperone-DNA replication initiator proteins expressed in (b) to its corresponding DNA construct by binding of the DNA replication initiator protein to the target sequence in the corresponding DNA construct, thereby producing the DRP CIS display library.

2. The method of embodiment 1, wherein the DRP has four or more cysteine residues for forming two or more disulfide bonds.

3. The method of embodiment 1 or embodiment 2, wherein each of the plurality of DNA constructs comprise a DNA sequence encoding a different DRP.

4. The method of any one of embodiments 1 to 3, wherein the encoded DRP comprises a natural and/or synthetic DRP.

5. The method of any one of embodiments 1 to 4, wherein the encoded DRP comprises an antibody, an scFv fragment, a cyclotide, a knottin, a toxin, and a cow CDR3 knob domain.

6. The method of any one of embodiments 1 to 5, wherein the encoded DRPs comprise DRP variants comprising one or more differences in their amino acid residues.

7. The method of any one of embodiments 1 to 6, wherein the one or more encoded molecular chaperones comprise a bacterial or eukaryotic origin.

8. The method of any one of embodiments 1 to 7, wherein the one or more encoded molecular chaperones comprise a molecular weight of 10 kD to 80 kD.

9. The method of any one of embodiments 1 to 7, wherein the one or more encoded molecular chaperones comprise a molecular weight of 10 kD to 24 kD.

10. The method of any one of embodiments 1 to 9, wherein the one or more encoded molecular chaperones comprise DsbA, DsbB, DsbC, trxA, HSP70, and/or DnaK.

11. The method of any one of embodiments 1 to 10, wherein the one or more molecular chaperone is the molecular chaperone DsbA.

12. The method of any one of embodiments 1 to 10, wherein the one or more molecular chaperone is the molecular chaperone trxA.

13. The method of any one of embodiments 1 to 12, wherein each DRP of the CIS display library has formed two or more disulfide bonds.

14. The method of any one of embodiments 1 to 13, wherein the DNA construct encodes the DRP, the one or more molecular chaperones, the DNA replication initiator protein, and the target sequence in-frame.

15. The method of any one of embodiments 1 to 14, wherein the one or more encoded molecular chaperones are encoded upstream and/or downstream of the DRP in the DNA construct.

16. The method of any one of embodiments 1 to 15, wherein the one or more encoded molecular chaperones are encoded upstream of the DRP in the DNA construct.

17. The method of any one of embodiments 1 to 16, wherein each of the plurality of DNA constructs comprises in order: the DNA sequence encoding the molecular chaperone, the DNA sequence encoding the DRP, the DNA sequence encoding the DNA replication initiator protein, and the DNA sequence encoding the target sequence for the DNA replication initiator protein.

18. The method of any one of embodiments 1 to 17, wherein each of the plurality of DNA constructs further comprises a first linker sequence between the molecular chaperone and the DRP and/or a second linker sequence between the DRP and the DNA replication initiator protein.

19. The method of embodiment 18, wherein the first linker is SEQ ID NO: 15.

20. The method of embodiment 18, wherein the second linker is SEQ ID NO: 15.

21. A polynucleotide comprising:
(a) a nucleic acid sequence encoding a disulfide rich protein (DRP);
(b) a nucleic acid sequence encoding one or more molecular chaperone;
(c) a nucleic acid sequence encoding a DNA replication initiator protein; and
(d) a nucleic acid sequence encoding a target sequence for the DNA replication initiator protein, wherein the DNA replication initiator protein can non-covalently bind to the target sequence.

22. The polynucleotide of any one of embodiment 21, wherein the nucleic acid sequence encoding the DRP has four or more cysteine residues for forming two or more disulfide bonds.

23. The polynucleotide of any one of embodiment 21 or embodiment 22, wherein the nucleic acid sequence encoding the DRP comprises natural and/or synthetic DRPs.

24. The polynucleotide of any one of embodiments 21 to 23, the nucleic acid sequence encoding the DRP comprises an antibody, an scFv fragment, a cyclotide, a knottin, a toxin, and a cow CDR3 knob domain.

25. The polynucleotide of any one of embodiments 21 to 24, wherein the nucleic acid sequence encoding the DRP comprises sequence variants that encode one or more differences in amino acid residues.

26. The polynucleotide of any one of embodiments 21 to 25, wherein the polynucleotide encodes the DRP sequence, the one or more molecular chaperone sequence, the DNA replication initiator protein sequence, and the target sequence in-frame.

27. The polynucleotide of any one of embodiments 21 to 26, wherein the one or more molecular chaperone nucleic acid sequences are upstream or downstream of the DRP nucleic acid sequence in the polynucleotide.

28. The polynucleotide of any one of embodiments 21 to 27, wherein the one or more molecular chaperone nucleic acid sequences are upstream of the DRP nucleic acid sequence in the polynucleotide.

29. The polynucleotide of any one of embodiments 21 to 28, wherein the one or more molecular chaperone nucleic acid sequences comprise a bacterial or eukaryotic origin.

30. The polynucleotide of any one of embodiments 21 to 29, wherein the one or more molecular chaperone comprises a molecular weight of 10 kD to 80 kD.

31. The polynucleotide of any one of embodiments 21 to 30, wherein the one or more molecular chaperone comprises a molecular weight of 10 kD to 24 kD.

32. The polynucleotide of any one of embodiments 21 to 31, wherein the one or more molecular chaperone comprises DsbA, DsbB, DsbC, trxA, HSP70, and/or DnaK.

33. The polynucleotide of any one of embodiments 21 to 32 wherein the one or more molecular chaperone sequences are the molecular chaperone DsbA.

34. The polynucleotide of any one of embodiments 21 to 33, wherein the one or more molecular chaperone sequences are the molecular chaperone trxA.

35. The polynucleotide of any one of embodiments 21 to 34, wherein the polynucleotide comprises in order: the nucleic acid sequence encoding the molecular chaperone, the nucleic acid sequence encoding the DRP, the nucleic acid sequence encoding the DNA replication initiator protein, and the nucleic acid sequence encoding the target sequence for the DNA replication initiator protein.

36. The polynucleotide of any one of embodiments 21 to 35, wherein the polynucleotide further comprises a first linker sequence between the molecular chaperone and the DRP and/or a second linker sequence between the DRP and the DNA replication initiator protein.

37. The polynucleotide of embodiment 36, wherein the first linker is SEQ ID NO: 15.

38. The polynucleotide of embodiment 36, wherein the second linker is SEQ ID NO: 15.

39. A DNA construct comprising the polynucleotide of embodiments 21 to 38.

40. An expression vector comprising the polynucleotide of embodiments 21 to 38.

41. A DNA:protein fusion comprising:
(a) a protein comprising:
   i. a disulfide rich protein (DRP);
   ii. one or more molecular chaperone proteins; and
   iii. a DNA replication initiator protein; and
(b) a DNA comprising:
   i. a DNA sequence encoding the DRP;
   ii. one or more DNA sequences encoding the molecular chaperone;
   iii. a DNA sequence encoding the DNA replication initiator protein; and
   iv. a DNA sequence encoding a target sequence for the DNA replication initiator protein;
wherein the DNA replication initiator protein non-covalently binds to the DNA sequence encoding the target sequence, thereby producing the DNA:protein fusion.

42. The DNA:protein fusion of embodiment 41, wherein the DRP comprises a natural and/or synthetic DRP.

43. The DNA:protein fusion of embodiment 41 or embodiment 42, wherein the DRP comprises an antibody, an scFv fragment, a cyclotide, a knottin, a toxin, and a cow CDR3 knob domain.

44. The DNA:protein fusion of any one of embodiments 41 to 43, wherein the one or more molecular chaperones comprise a bacterial or eukaryotic origin.

45. The DNA:protein fusion of any one of embodiments 41 to 44, wherein the one or more molecular chaperones comprise a molecular weight of 10 kD to 80 kD.

46. The DNA:protein fusion of any one of embodiments 41 to 45, wherein the one or more molecular chaperones comprise a molecular weight of 10 kD to 24 kD.

47. The DNA:protein fusion of any one of embodiments 41 to 46, wherein the one or more molecular chaperones comprise DsbA, DsbB, DsbC, trxA, HSP70, and/or DnaK.

48. The DNA:protein fusion of any one of embodiments 41 to 47, wherein the one or more molecular chaperone is the molecular chaperone DsbA.

49. The DNA:protein fusion of any one of embodiments 41 to 47, wherein the one or more molecular chaperone is the molecular chaperone trxA.

50. The DNA:protein fusion of any one of embodiments 41 to 49, wherein the DRP protein has formed two or more disulfide bonds.

51. The DNA:protein fusion of embodiments 41 to 50, wherein the one or more molecular chaperones are encoded upstream and/or downstream of the DRP in the DNA.

52. The DNA:protein fusion of any one of embodiments 41 to 51, wherein the one or more molecular chaperones are encoded upstream of the DRP in the DNA.

53. The DNA:protein fusion of any one of embodiments 41 to 52, wherein the DNA comprises in order: the DNA sequence encoding the molecular chaperone, the DNA sequence encoding the DRP, the DNA sequence encoding the DNA replication initiator protein, and the DNA sequence encoding the target sequence for the DNA replication initiator protein.

54. The DNA:protein fusion of any one of embodiments 41 to 53, wherein the DNA further comprises a first linker sequence between the molecular chaperone and the DRP and/or a second linker sequence between the DRP and the DNA replication initiator protein.

55. The DNA:protein fusion of embodiment 54 wherein the first linker is SEQ ID NO: 15.

56. The DNA:protein fusion of embodiment 54, wherein the second linker is SEQ ID NO: 15.

57. A screening library produced according to the method of embodiments 1 to 20, comprising a DNA:protein fusion comprising a DRP-chaperone-DNA replication initiator protein bound to the corresponding DNA construct encoding said DRP-chaperone-DNA replication initiator protein sequence.

58. The screening library of embodiment 57 comprising 10⁶ to 10¹⁴ different DNA:protein fusions.

59. The screening library of embodiment 57 comprising 10¹⁰ to 10¹⁴ different DNA:protein fusions.

60. A method of screening a disulfide rich protein (DRP) CIS display library, the method comprising:
(a) providing a screening library according to embodiments 1 to 20 or embodiments 41 to 59, wherein each protein of a plurality of DRP-chaperone-DNA replication initiator proteins is linked to its corresponding DNA sequence;
(b) contacting the screening library with a target of interest to produce a mixture of DRP-chaperone-DNA replication initiator protein DNA:protein fusions and the target of interest;
(c) selecting from the mixture the DRP-chaperone-DNA replication initiator protein DNA:protein fusions that are bound to the target of interest.

61. The method of embodiment 60, further comprising identifying the one or more DRP proteins bound to the target of interest by identifying a sequence of the corresponding DRP-chaperone-DNA replication initiator protein DNA.

62. The method of embodiments 60 or embodiment 61, wherein the one or more encoded molecular chaperones comprise a bacterial or eukaryotic origin.

63. The method of any one of embodiments 60 to 62, wherein the one or more encoded molecular chaperones comprise a molecular weight of 10 kD to 80 kD.

64. The method of any one of embodiments 60 to 63, wherein the one or more encoded molecular chaperones comprise a molecular weight of 10 kD to 24 kD.

65. The method of any one of embodiments 60 to 64, wherein the one or more encoded molecular chaperones comprise DsbA, DsbB, DsbC, trxA, HSP70, and/or DnaK.

66. The method of any one of embodiments 60 to 65, wherein the one or more molecular chaperone is the molecular chaperone DsbA.

67. The method of any one of embodiments 60 to 65, wherein the one or more molecular chaperone is the molecular chaperone trxA.

68. The method of any one of embodiments 60 to 67, wherein each DRP of the CIS display library has formed two or more disulfide bonds.

69. The method of any one of embodiments 60 to 68, further comprising screening the DRP CIS display library by contacting the expressed plurality of DNA constructs with a lysate comprising one or more molecular chaperones.

70. The method of any one of embodiments 60 to 69, wherein identifying the sequence of the corresponding DRP-chaperone-DNA replication initiator protein DNA comprises nucleic acid sequencing.

### Examples

The following examples are included for illustrative purposes only and are not intended to limit the scope of the invention.

### Example 1: Recovery of Functional Disulfide-Rich Protein-DNA Complexes by Fusion to a Chaperone Protein in a CIS Display System

An in vitro CIS display system is not compatible for screening DRPs, as the in vitro assay is performed in a reducing environment. This reducing environment prevents the correct formation of disulfide bonds, resulting in instability and aggregation of DRPs, proteins which include two or more disulfide bonds. To overcome the system's limitations, chaperone proteins were included. In this Example, the disulfide-rich protein cow CDR3 knobs were expressed and displayed using in vitro DNA-based CIS display systems. The functionality of DRPs was tested after expressing using the CIS display systems, including the improved CIS display system. Cow CDR3 knob protein-DNA complexes, expressed from constructs lacking or encoding chaperone proteins, were collected to assess binding activity of cow CDR3 proteins to their cognate targets.

### A. Expression, Display, and Binding Assessment of Disulfide-Rich Proteins

Two exemplar DRPs and chaperone proteins were cloned and expressed using the in vitro CIS display system. Conditions encoding the exemplar DRPs using CIS display systems with (FIG. 1A) and without (FIG. 1B) chaperone proteins, (e.g., trxA, DsbA, DsbB, or DsbC) were evaluated to assess the impact of chaperones on the functionality of displayed DRPs.

Two cow CDR3 knobs (i.e., exemplar DRPs) linked to the p53 DO1 antibody epitope were cloned: 1) SARS2 spike trimer binding R4C1-DO1 (hereinafter referred to as ‘R4C1’; SEQ ID NOS: 2, 3) and 2) SARS2 spike RBD binding R2G3-DO1 (hereinafter referred to as ‘R2G3’; SEQ ID NOS: 4, 5) (Huang et al. Proc Natl Acad Sci USA. 2023; 120 (39):e2303455120). SARS2 spike trimer contains three disulfide bonds and SARS2 spike RBD contains four disulfide bonds. DNA sequences encoding the DRPs sequence were cloned into a vector encoding the CIS display system in the ‘Library’ portion (FIG. 1). An exemplar vector, pREPTRX (FIG. 2, SEQ ID NO: 1) depicts the key sites and components of the CIS display system including a chaperone protein (FIG. 1A). In preparation for cloning of the CIS display vector, the aforementioned DRPs were amplified with primer pairs ‘TRXA2FOR’ and ‘NOTRECREV4’ listed in Table 1. Vector pREPTRX was digested with the restriction enzymes NcoI and PspOMI. The amplified fragments were ligated as NcoI-NotI fragments into the digested pREPTRX vector (‘NcoI-PspOMI’) in-frame with the chaperone gene (i.e., trxA) and the RepA-CIS-ORI DNA elements. The amplified fragments were also similarly cloned into a digested pREP vector, which does not include a chaperone gene (‘trxA’), and separately amplified from the four ligations with FOR1-ORIREV primers listed in Table 1.

Cloned vectors were further expressed and displayed in vitro. First, vectors encoding the DRP-chaperone-RepA fusion, R4C1-trxA-RepA and R2G3-trxA-RepA, were mixed at a 1:1 ratio. Vectors encoding the DRP-RepA fusion without the chaperone, R4C1-RepA and R2G3-RepA, were also mixed at a 1:1 ratio. One μg of each pooled DNA mixture was subjected to in vitro transcription/translation in a 50 μl volume using a Promega linear template S30 reaction system kit at 25° C. for 1 hour. Each reaction was mixed with 1.5 ml of blocking buffer (2% milk powder/PBS/100 μg/ml heparin/100 μg/ml salmon sperm DNA) and blocked for 1 hour at room temperature to reduce non-specific binding for downstream binding assessment.

Further, to assess binding of DRPs to the target spike protein and receptor binding domain, 500 μl of each reaction was added to NUNC-Immuno tubes coated with 1 ml of either anti-DO1 antibody, SARS2 full length spike protein, or SARS2 RBD protein, all at a 1 μg/ml concentration. The in vitro transcribed/translated products (Mix A: ‘R4C1-RepA’ and ‘R2G3-RepA’; Mix B: ‘R4C1-trxA-RepA’ and ‘R2G3-trxA-RepA’) were incubated with the targets for 1 hour at room temperature. After, all tubes were extensively washed with 10× with PBS/0.1% Tween 20 and then 10×PBS to remove unbound DNA-protein complexes. Bound DNA was recovered by incubating the reaction with 0.6 ml Qiagen PB buffer on a blood mixer for 10 minutes and purified using one Qiagen PCR purification column per binding reaction and recovered DNA was eluted in 100 μl of water.

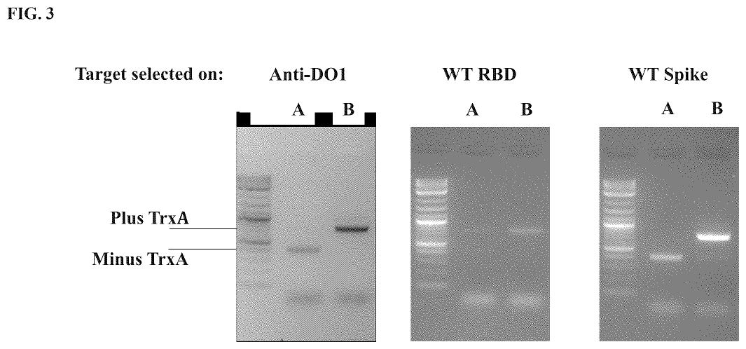

A portion (10%) of the recovered DNA was used for PCR amplification using TACFAR6.2 and NOTRECREV4 primers for 25 cycles and visualized on a 1% agarose/TAE gel (FIG. 3). Products bound to anti-DO1, WT RBD, or WT Spike, were visualized on an agarose gel. Both R2G3 and R4C1 PCR products are too similar in size to separate under these conditions. However, the intensity of the bands between the samples (e.g., lanes ‘A’ and ‘B’) with either the inclusion or exclusion of a chaperone protein can inform regarding the effect of a chaperone protein on the binding affinity of the encoded protein. Amplified products with a chaperone protein (e.g., TrxA; FIG. 3 lanes ‘B’) have an enhanced recovery of the binding protein-DNA complexes. For example, products selected with WT RBD are only visible with the mixture containing ‘R4C1-trxA-RepA’ and ‘R2G3-trxA-RepA’. Further, these products are from the DNA-protein complexes comprising R2G3-trxA-RepA as only R2G3 can bind to the RBD protein. There is also a significant recovery improvement for products selected with the spike protein (FIG. 3, ‘WT Spike’ gel). This example demonstrates the use of chaperone proteins to improve DRPs functionality, as the inclusion of a chaperone (e.g., trxA) DNA-protein fusion in a CIS display system resulted in correctly folded disulfide proteins capable of binding to their cognate target.

TABLE 1
PCR Primers for cloning CIS display vectors.

| | | SEQ |
|---|---|---|
| | | ID |
| Primer Name | Sequence 5′ - 3′ | No. |
| igGCDNAREV | CCGCTCTTCAGGGCACCCGAGTTCC | 16 |
| igACDNAREV | GACACGCTGTCGCCATTCTGGTTCC | 17 |
| p2pET32cloning | aaaaaaCTCGAGTCACCAGGCATCGACGTAGAATTC | 18 |
| p6PET32cloning | aaaaaaCTCGAGTCACCAGGCATCGACGAGCCATTG | 19 |
| p7pET32cloning | aaaaaaCTCGAGTCACCAGGCATCGACGTGCCATTC | 20 |
| CISXHOISTOPR | gtaatactcgagTCAGTGAAGATCAGTTGGGGCCgCTAG | 21 |
| ECREV | | |
| G3LIBPTFOR | GGTTCTGGCGCCATGGCCGGCTCCGAAGG | 22 |
| G3LIBPTREV | ATCAGTTGCGGCCGCTAGAGACCCACCACCACCCTCGAGG | 23 |
| | TCACTAAGCCAACCACCC | |
| FOR1 | GCTCTTCAGCAATATCACGGGTAGCCAACGCTATGTCC | 24 |
| FOR2 | CGGTCCGGACGTTGTAAAACGACGGCCAGTGAATTG | 25 |
| FOR3 | CaGCCGCGcATTCGCCCTTAACCGACTCTGACGGC | 26 |
| FOR4 | ACGAGAGAGATGATAGGGTCTGCTTCAGTAAGCCAG | 27 |
| TACFAR6.2 | ATGCTACACAATTAGGCTTGTAC | 28 |
| NOTRECREV4 | TGGTGAAGATCAGTTGCGGCCGCTAG | 29 |
| ORIREV | TGCATATCTGTCTGTCCACAGG | 30 |
| BSPREPAFOR | GCATCAAGGGCCCCAACTGATCTTCACCAAACGTATTACC | 31 |
| BovStalkFor2 | CAGCCGGCCATGGCCACATACTACAGTACTACTGTATACC | 32 |
| BovStalkFor3 | CAGCCGGCCATGGCCACATACTACAGTACTACTGTGCTCC | 33 |
| BovStalkFor4 | CAGCCGGCCATGGCCACATACTACAGTGGTACTGTGCACC | 34 |
| CISCOWREV2 | ttttttGCGGCCGCTAGAGAACCACCCCAGGCATCGACGTAGA | 35 |
| | ATTC | |
| CISCOWREV6 | ttttttgcggccgctagagaaccaccCCAGGCATCGACGAGCCATTG | 36 |
| CISCOWREV7 | ttttttgcggccgctagagaaccaccCCAGGCATCGACGTGCCATTC | 37 |

Example 2: Improved Binding of a Functional scFv Antibody Using a CIS Display System with Chaperone Proteins

Disulfide-rich proteins (DRPs), including an antibody scFv fragment specific for αvβ3 integrin (SEQ ID NO: 6) and a cow CDR3 knob, were expressed and displayed using an improved in vitro DNA-based CIS display system comprising chaperone proteins. The impact of chaperone proteins on the functionality of DRPs was tested by collecting expressed DRP-chaperone-DNA complexes to assess their binding activity to cognate targets.

### A. Expression, Display, and Binding Assessment of Disulfide-Rich Proteins

Two exemplar DRPs were cloned and expressed using the in vitro CIS display system comprising chaperone proteins (FIG. 1A). DNA sequences encoding the DRPs sequence were cloned into a vector encoding the CIS display system in the ‘Library’ portion (FIG. 1). First, an antibody scFv fragment specific for αvβ3 integrin (SEQ ID NO: 6) was amplified with TRXA2FOR and NOTRECREV4 primer pairs listed in Table 1. Vector pREPTRX was digested with the restriction enzymes NcoI and PspOMI. The amplified fragments were ligated as NcoI-NotI fragments into the digested pREPTRX vector (‘NcoI-PspOMI’) in-frame with the chaperone gene (e.g., trxA) and the RepA-CIS-ORI DNA elements. The R2G3-trxA-RepA fusion was also cloned as described in Example 1.

Cloned vectors were further expressed and displayed in vitro. First, the vector encoding the αvβ3 integrin scFv-trxA-RepA fusion (10 ng) were mixed at a 1:100 ratio with 1 μg of R2G3-trxA-RepA fusion DNA vector. The pooled DNA mixture was subjected to in vitro transcription/translation in a 50 μl volume using a Promega linear template S30 reaction system kit at 25° C. for 1 hour. The reaction was mixed with 1 ml of blocking buffer (2% milk powder/PBS/100 μg/ml heparin/100 μg/ml salmon sperm DNA) and blocked for 1 hour at room temperature to reduce non-specific binding for downstream binding assessment.

Further, to assess binding of DRPs to the target integrin and receptor binding domain, 500 μl of each reaction was added to NUNC-Immuno tubes coated with 1 ml of either αvβ3 integrin protein (R&D Systems) or SARS2 RBD protein at 1 μg/ml. The in vitro transcribed/translated products (‘scFv-trxA-RepA’ and ‘R2G3-trxA-RepA’) were incubated with the targets for 1 hour at room temperature. After, all tubes were extensively washed with 10× with PBS/0.1% Tween 20 and then 10×PBS to remove unbound DNA-protein complexes. Bound DNA was recovered by incubating the reaction with 0.6 ml Qiagen PB buffer on a blood mixer for 10 minutes and purified using one Qiagen PCR purification column per binding reaction and recovered DNA was eluted in 100 μl of water.

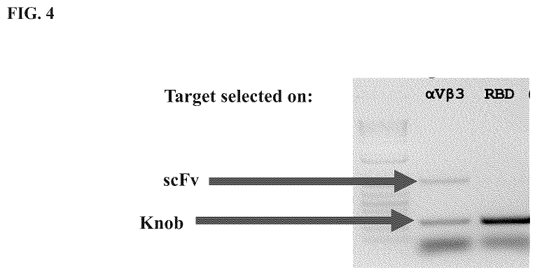

A portion (10%) of the recovered DNA was used for PCR amplification using TACFAR6.2 and NOTRECREV4 primers for 25 cycles and visualized on a 1% agarose/TAE gel (FIG. 4).

Products bound to αvβ3 or RBD were visualized on an agarose gel. The intensity of the bands between the samples (e.g., lanes ‘αvβ3’ and ‘RBD’) for scFv or knob proteins provides information regarding the effect of chaperone proteins on the binding affinity of the encoded proteins. The inclusion of a chaperone protein (e.g., trxA) improved the recovery of specific binding protein-DNA complexes (FIG. 4). For example, the recovery of knob products was improved when selected with WT RBD vs. when using integrin selection (FIG. 4; ‘knob’ band). Additionally, the recovery of scFv products was only visible when integrin selection was used (FIG. 4; ‘scFv’ band). This example demonstrates the use of chaperone proteins to improve DRPs binding specificity, as the inclusion of DNA-chaperone (e.g., trxA)-protein fusion in the CIS display system resulted in specific enrichment of DRPs capable of binding to their cognate target.

Example 3: Selection of Disulfide-Rich Microproteins from a Variant DRP Library Using a CIS Display System with Chaperone Proteins

A library of variant DRPs based on a cow CDR3 knob DRP (e.g., R2G3) binding to the SARS CoV2 spike RBD protein was screened using an improved in vitro DNA-based CIS display system comprising chaperone proteins. After multiple rounds of screening and selection, selected clones were expressed, purified, and assayed for their ability to bind to the SARS CoV2 spike protein (e.g., RBD). Binding of the library candidates to RBD was assessed via ELISA. Lastly, the sequence information of the selected DRP variants was compared to the measured binding values to confirm the enrichment and selection of relevant DRP variants.

### A. R2G3 Cow CDR3 Knob Library Construction and Initial Selection

A library of variant DRP proteins based on the SARS CoV2 spike RBD binding R2G3 cow CDR3 knob was cloned using the in vitro CIS display system comprising chaperone proteins (FIG. 1A). DNA sequences encoding the DRPs sequence were cloned into a vector encoding the CIS display system in the ‘Library’ portion (FIG. 1). A R2G3 cow CDR3 knob variant library of NNB oligonucleotides (Gene Link Inc. USA) was synthesized encoding the library in SEQ ID NO: 7. The oligonucleotides were used as templates to amplify with primers ‘G3LIBPTFOR’ and ‘G3LIBPTREV’ listed in Table 1. Vector pREPTRX was digested with the restriction enzymes NcoI and PspOMI. The amplified library DNA fragments were ligated as NcoI-NotI fragments into the digested pREPTRX vector (‘NcoI-PspOMI’) in-frame with the chaperone gene (e.g., trxA) and the RepA-CIS-ORI DNA elements. Lastly, the ligated products were PCR amplified with FOR1-ORIREV primers (Table 1).

Three μg of the DNA fragments encoding the variant DRP library were subjected to in vitro transcription/translation in a 100 μl volume using a Promega linear template S30 reaction system kit at 25° C. for 1 hour according to the manufacturer's instructions. After in vitro transcription/translation, the reaction was mixed with 1 ml of blocking buffer (2% milk powder/PBS/100 μg/ml heparin/100 μg/ml salmon sperm DNA). Further, to assess binding of DRPs to the target SARS2 Delta RBD protein, the reaction was added to NUNC-Immuno tubes coated with 1 ml of 10 μg/ml SARS2 Delta RBD protein in PBS overnight at 4° C. Prior to the addition of the reaction, the NUNC-Immuno tubes were blocked for 1 hour at room temperature to reduce non-specific binding for downstream binding assessment. After incubating for 1 hour at room temperature, the tubes were extensively washed with 10× with PBS/0.1% Tween 20 and then 10×PBS to remove unbound DNA-protein complexes. Bound DNA was recovered by incubating the reaction with 0.6 ml Qiagen PB buffer on a blood mixer for 10 minutes and purified using one Qiagen PCR purification column per binding reaction and recovered DNA was eluted in 100 μl of water.

### B. R2G3 Cow CDR3 Knob Library Selection

After the initial recovery, the library was prepared for second round of selection. A portion (50%) of the recovered eluted DNA was used for PCR amplification using FOR2 and NOTRECREV4 primers in 2×50 μl PCR reactions. The amplification products were gel purified using an NEB Monarch gel purification kit. RepA-CIS-ORI DNA elements were reattached to the eluted DNA using a restriction-ligation reaction of 200 ng of BSPREPAFOR-ORIREV amplified DNA, which was incubated for 2 hours at 37° C. (reaction details can be found in Table 2).

After the restriction ligation reaction was completed, 20 μl of the restriction-ligation reaction was PCR amplified in 4×50 μl pull through reactions using primers ‘FOR2’ and ‘ORIREV’ (Table 1). The amplified products were purified with Qiagen PCR purification columns to generate fresh templates for the second round of selection. The second and third rounds of selection was carried out as described in round 1 above, with a couple exceptions. The recovery PCR was carried out using primers ‘FOR3’ and ‘NOTRECREV4’ in round two, and pull through reaction primers used were ‘FOR3’ and ‘ORIREV’ (Table 1). After the third round of selection, recovery PCR was amplified with ‘G3LIBPTFOR’ and ‘CISXHOISTOPRECREV’ primers, and cloned via restriction ligation into pET32 vector as NcoI-XhoI fragments producing trxA fusions, or it was amplified with ‘TRXA2FOR’ and ‘CISDNGSREPAREV’ primers and selected DNA sequences were analyzed by next-generation sequencing (NGS; Azenta).

TABLE 2
Restriction-ligation Reaction Components.

| Component | Amount |
|---|---|
| 10xNEB buffer 4 | 10 μl |
| 100 mM ATP | 2 μl |
| 200 ng RepA DNA | 2 μl |
| Selected/recovered DNA | 50 μl |
| PspOMI (10 μ/μl NEB) | 2 μl |
| NotI (10 μ/μl NEB) | 2 μl |
| T4 DNA ligase (400 μ/μl NEB) | 2 μl |
| Water | 30 μl |

### C. Binding Assessment of R2G3 Cow CDR3 Knob Library Candidates to RBD

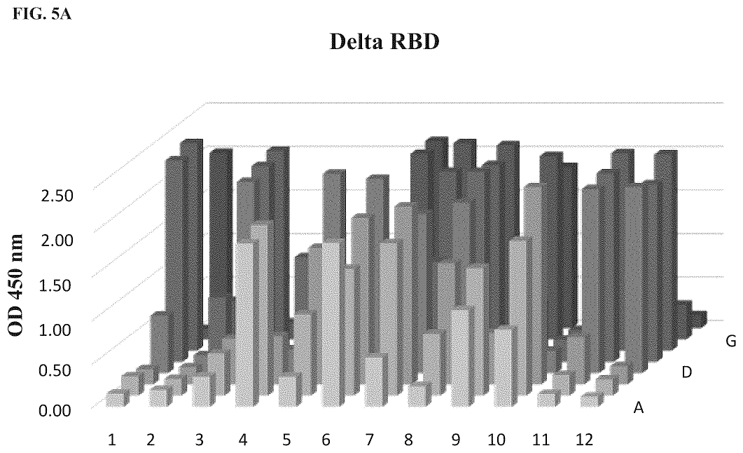

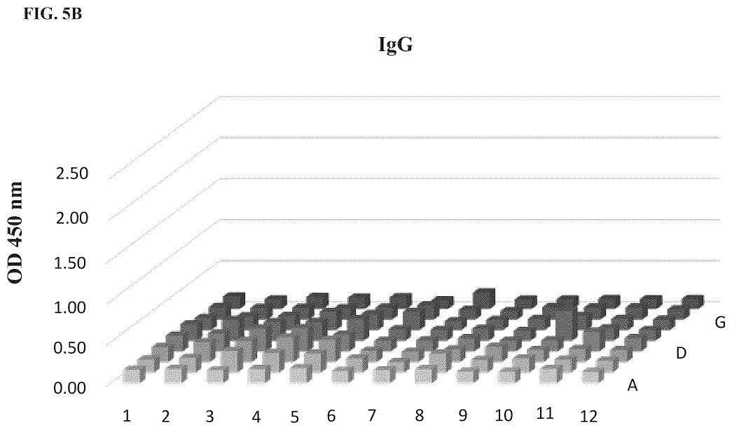

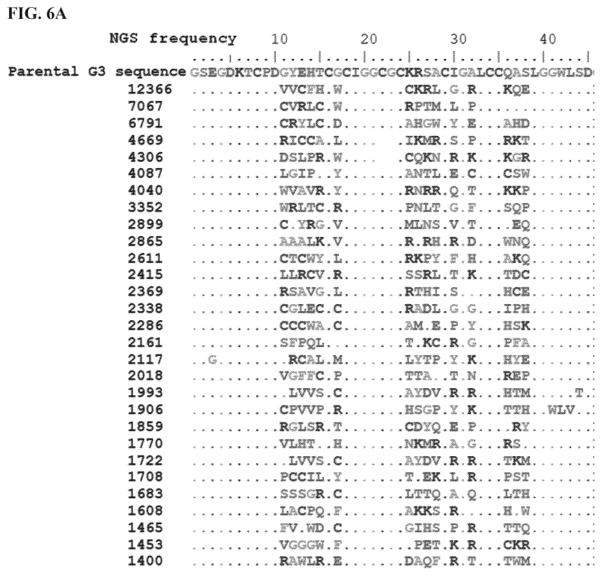

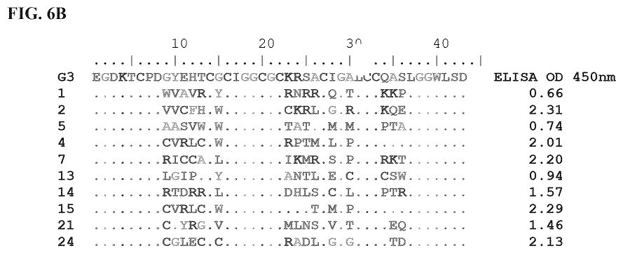

Individual clones remaining after the third round of selection were screened using ELISA. The pET32 ligation comprising the DRP-trxA-RepA cassette was used to transform Origami E. coli competent cells. Clones were selected using 1 ml 2×YT/2% glucose/50 μg/ml carbenicillin in 96-well deep well plates, grown overnight in a shaking incubator at 37° C., then grown in fresh plates with the same medium for 4-5 hours. The inoculates were spun to pellet the bacteria, and 1 ml of fresh 2× YT/50 μg/ml carbenicillin/0.6 mM IPTG was added. Incubation continued overnight in a shaking incubator at 25° C. After the overnight incubation, bacteria were pelleted, and the pellets were lysed in 100 μl Bugbuster HT for 30 minutes at room temperature. The lysate was cleared by centrifugation. The cleared lysate (25 μl) was mixed with an equal volume of 4% milk powder/PBS and screened for binding to either 1 μg/ml Delta RBD or human IgG as a negative control protein. The plates had been previously coated overnight in 50 μl/well PBS at 4° C., then blocked for 1 hour at room temperature with 100 μl/well 2% milk powder/PBS. Each plate was washed two times with 200 μl/well PBS/0.1% Tween 20, then two times with 200 μl/well PBS. Bound fusion protein was detected using 50 μl/well of 1:10,000 diluted anti-S-tag-HRP conjugate (Fisher Scientific) in 2% milk powder/PBS for 1 hour at room temperature and the plate was further washed two times with 200 μl/well PBS/0.1% Tween 20, and lastly two times with 200 μl/well PBS. The plates were developed for 5-10 minutes at room temperature with 50 μl/well TMB (3,3′,5,5′-Tetramethylbenzidine) substrate buffer, and washed once with 200 μl/well PBS/0.1% Tween 20, and then washed two times with 200 μl/well PBS. The reaction was stopped using 50 μl/well 0.5N H₂SO₄ and the signal was read at 450 nm. Clones with binding to the target were observed (FIG. 5A). Minimal non-specific binding was observed using the negative control IgG (FIG. 5B). Individual clones with observed binding were sequenced and the NGS sequence output was compared (FIG. 6A). Representative sequences and ELISA binding values (OD 450 nm) for individual clones after three rounds of selection were also compared (FIG. 6B). The most frequently detected NGS clone sequences were shown to be specific binders, demonstrating successful selection of novel DRP clone sequences using a DRP variant library expressed by an in vitro CIS display system comprising chaperone proteins (FIG. 6).

Example 4: Selection of MERS Spike Trimer Disulfide-Rich Microproteins from a Variant DRP Library Using a CIS Display System with Chaperone Proteins

A library of CDR3 knob trxA fusions was generated and screened using an in vitro DNA-based CIS display system comprising chaperone proteins. After multiple rounds of screening and selection, selected clones were expressed, purified, and assayed for their ability to bind MERS spike trimer protein. Binding of the library candidates to MERS spike trimer protein was assessed via ELISA. Lastly, sequence information of selected DRP candidates was obtained.

To generate the library, peripheral blood mononuclear cells (PBMCs) were collected from immunized cows and RNA was extracted and used to generate a CDR3 knob trxA fusion library as described below. Specifically, approximately 1-5×10⁷ PBMCs were collected after 14-64 days post-immunization and RNA was isolated using an RNAeasy kit (Qiagen). Immune cow antibody variable heavy (VH) regions repertoires were obtained through cDNA synthesis from 5 μg total RNA using Superscript IV First-Strand cDNA synthesis kit (ThermoFisher, #18091050), the cDNA template for VHs were synthesized using a pool of IgA and IgG-specific primers (Primers: igGcDNAREV and igAcDNAREV, listed in Table 1). Primary CDR3 stalk-knobs were amplified from 1st strand cDNA with IgHV1-7 family specific primers (BOVSTALK2,3,4 and CISCOWREV2, 6 and 7 listed in Table 1). These primers are specific for either side of the CDR3 knob region and were used to enrich for ultralong CDR3 regions. Vector pREPTRX was digested with restriction enzymes NcoI-PspOMI. Library DNA fragments were ligated in-frame as NcoI-NotI fragments into digested pREPTRX vector in-frame with trxA and RepA-CIS-ORI DNA elements, which were PCR amplified from the ligation with FOR1-ORIREV primers (Table 1).

In the first round of selection, three μg of the VH CDR3 DNA fragments encoding the variant DRP library were subjected to in vitro transcription/translation in a 100 μl volume using a Promega linear template S30 reaction system kit at 25° C. for 1 hour, according to the manufacturer's instructions. After in vitro transcription/translation, the reaction was mixed with 1 ml of blocking buffer (2% milk powder/PBS/100 μg/ml heparin/100 μg/ml salmon sperm DNA). Further, to assess binding of DRPs to the target MERS virus spike trimer protein, the reaction was added to NUNC-Immuno tubes coated with 1 ml of 10 μg/ml MERS virus spike trimer protein in PBS overnight at 4° C. Prior to the addition of the reaction, the NUNC-Immuno tubes were blocked for 1 hour at room temperature to reduce non-specific binding. After incubating for 1 hour at room temperature, the tubes were extensively washed with 10× with PBS/0.1% Tween 20 and then 10×PBS to remove unbound DNA-protein complexes. Bound DNA was recovered by incubating the reaction with 0.6 ml Qiagen PB buffer on a blood mixer for 10 minutes and purified using one Qiagen PCR purification column per binding reaction and recovered DNA was eluted in 100 μl of water.

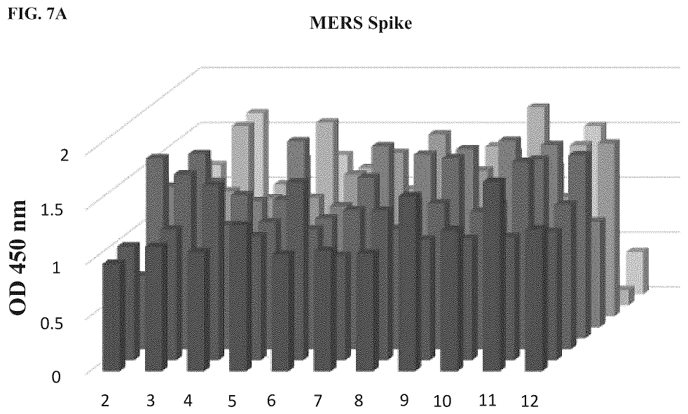

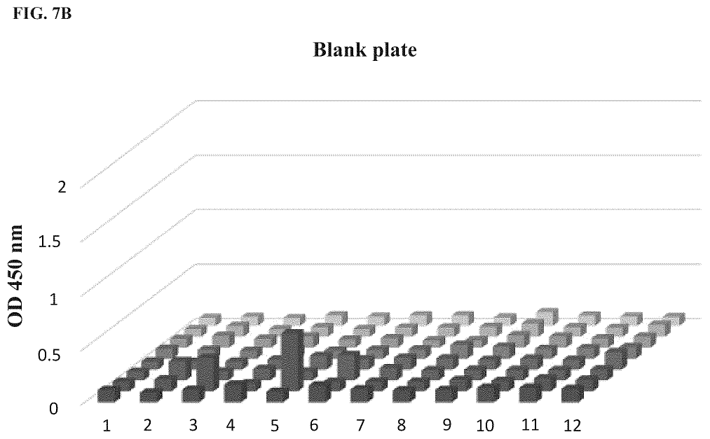

The second and third round of selection were carried out as described above in Example 3. After the third round of selection, the recovery PCR was amplified with primers BovStalkFor2, BovStalkFor3, BovStalkFor4, and p2pET32cloning, p6PET32cloning, and p7pET32cloning (e.g., see Table 1) and cloned into the pET32 vector with restriction-ligation cloning of NcoI-XhoI fragments as trxA fusions. Individual clones from the third-round output were screened via ELISA against MERS spike trimer protein as described in Example 3 and the binding data is shown in FIG. 7A. Almost every well represents a binding clone after 3 rounds of selection. Minimal non-specific binding was observed using the negative control blank plate (FIG. 7B). Representative sequences were obtained from positive wells and are shown in Table 3 below.

This example demonstrates the successful selection of novel DRP sequences that bind to MERS spike trimer protein by expressing a DRP variant library with an in vitro CIS display system comprising chaperone proteins (FIG. 7).

TABLE 3
Representative DRP sequences obtained after three rounds of
selection against MERS spike trimer protein.

| DRP | |
|---|---|
| Representative | DRP Sequences |
| G1 | QQTQRKQTCLDGSHLDDRCFGGPGCSTGDRWRCTTPIVTYNYEWL |
| | VDAW |
| D8 | QRDAHRCPDGYTVNSVCPSGCSRPIGGGVECANYGSGRDGVCTSET |
| | CRSSYEFYVDAW |
| A11(x12) | QKTEKSQSCPDGYAQAFDGLLGWGCRASNGYGPWSERIDTYTYEF |
| | YVDAW |
| C1(x13) | QKTKRKSCPDGCTDGSWSECYQPSWSIECGCCPVRPDDASVLRYEW |
| | LVDAW |
| G11 | GYTVNSVCPSGCSRPIGGGVECANYGSGRDGVCTSETCRSSYEFYV |
| | DAW |
| H1 | QKTTKSCPYGYTYHPRCWYGCRDVDKCRSYTYITIYEWHVDAW |
| B2(x33) | QKTRIKNCPDGCEDVPWNKCYLASWSSDCGCCPIRPAAASVEAHE |
| | WLVDAW |

The present disclosure is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

Sequences

| SEQ | | |
|---|---|---|
| ID NO. | Sequence | Description |
| 1 | CGGTCCGGACGTTGTAAAACGACGGCCAGTGAATT | DNA vector |
| | GTAATACGACTCACTATAGGGCGAATTGAATTTAGC | pREPTRX |
| | aGCCGCGcATTCGCCCTTAACCGACTCTGACGGCAGT | |
| | TTACGAGAGAGATGATAGGGTCTGCTTCAGTAAGCC | |
| | AGATGCTACACAATTAGGCTTGTACATATTGTCGTT | |
| | AGAACGCGGCTACAATTAATACATAACCTTATGTAT | |
| | CATACACATACGATTTAGGTGACACTATAGAATACA | |
| | AGCTTACTCCCCATCCCCCTGTTGACAATTAATCAT | |
| | GGCTCGTATAATGTGTGGAATTGTGAGCGGATAACA | |
| | ATTTCACACAGGAAACAGGATCTACCATGAGCGAT | |
| | AAAATTATTCACCTGACTGACGACAGTTTTGACACG | |
| | GATGTACTCAAAGCGGACGGGGCGATCCTCGTCGA | |
| | TTTCTGGGCAGAGTGGTGtGGTCCGTGCAAAATGAT | |
| | CGCCCCGATTCTGGATGAAATCGCTGACGAATATCA | |
| | GGGCAAACTGACCGTTGCAAAACTGAACATCGATC | |
| | AAAACCCTGGCACTGCGCCGAAATATGGCATCCGT | |
| | GGTATCCCGACTCTGCTGCTGTTCAAAAACGGTGAA | |
| | GTGGCGGCAACCAAAGTGGGTGCACTGTCTAAAGG | |
| | TCAGTTGAAAGAGTTCCTCGACGCTAACCTGGCCGG | |
| | TTCTGGTTCTGGCGCCATGGCCTAACGAGCTCatataaC | |
| | TCGAGggtggtggtgggtctCTAGCGGGCCCCAACTGATCTT | |
| | CACCAAACGTATTACCGCCAGGTAAAGAACCCGAA | |
| | TCCGGTGTTCACTCCCCGTGAAGGTGCCGGAACGCT | |
| | GAAGTTCTGCGAAAAACTGATGGAAAAGGCGGTGG | |
| | GCTTCACCTCCCGTTTTGATTTCGCCATTCATGTGGC | |
| | GCATGCCCGTTCCCGTGGTCTGCGTCGGCGCATGCC | |
| | ACCGGTGCTGCGTCGACGGGCTATTGATGCGCTGCT | |
| | GCAGGGGCTGTGTTTCCACTATGACCCGCTGGCCAA | |
| | CCGCGTCCAGTGTTCCATCACCACACTGGCCATTGA | |
| | GTGCGGACTGGCGACAGAGTCCGGTGCAGGAAAAC | |
| | TCTCCATCACCCGTGCCACCCGTGCCCTGACGTTCC | |
| | TGTCAGAGCTGGGACTGATTACCTACCAGACGGAAT | |
| | ATGACCCGCTTATCGGGTGCTACATTCCGACCGACA | |
| | TCACGTTCACACTGGCTCTGTTTGCTGCCCTTGATGT | |
| | GTCTGAGGATGCAGTGGCAGCTGCGCGCCGCAGTC | |
| | GTGTTGAATGGGAAAACAAACAGCGCAAAAAGCAG | |
| | GGGCTGGATACCCTGGGTATGGATGAGCTGATAGC | |
| | GAAAGCCTGGCGTTTTGTGCGTGAGCGTTTCCGCAG | |
| | TTACCAGACAGAGCTTAAGTCCCGTGGAATAAAAC | |
| | GTGCCCGTGCGCGTCGTGATGCGAACAGAGAACGT | |
| | CAGGATATCGTCACCCTGGTGAAACGGCAGCTGAC | |
| | GCGCGAAATCTCGGAAGGACGCTTCACTGCTAATG | |
| | GTGAGGCGGTAAAACGCGAAGTGGAGCGTCGTGTG | |
| | AAGGAGCGCATGATTCTGTCACGTAACCGCAATTAC | |
| | AGCCGGCTGGCCACAGCTTCTCCCTGAAAGTGATCT | |
| | CCTCAGAATAATCCGGCCTGCGCCGGAGGCATCCGC | |
| | ACGCCTGAAGCCCGCCGGTGCACAAAAAAACAGCG | |
| | TCGCATGCAAAAAACAATCTCATCATCCACCTTCTG | |
| | GAGCATCCGATTCCCCCTGTTTTTAATACAAAATAC | |
| | GCCTCAGCGACGGGGAATTTTGCTTATCCACATTTA | |
| | ACTGCAAGGGACTTCCCCATAAGGTTACAACCGTTC | |
| | ATGTCATAAAGCGCCAGCCGCCAGTCTTACAGGGTG | |
| | CAATGTATCTTTTAAACACCTGTTTATATCTCCTTTA | |
| | AACTACTTAATTACATTCATTTAAAAAGAAAACCTA | |
| | TTCACTGCCTGTCCTGTGGACAGACAGATATGCAAA | |
| | GGGCGAATTCGTTTAAACCTGCAGGACTAGTCCCTT | |
| | TAGTGAGGGTTAATTCTGAGCTTGGCGTAATCATGG | |
| | TCATAGCTGTTTCCTGTGTGAAATTGTTATCCGCTCA | |
| | CAATTCCACACAACATACGAGCCGGAAGCATAAAG | |
| | TGTAAAGCCTGGGGTGCCTAATGAGTGAGCTAACTC | |
| | ACATTAATTGCGTTGCGCTCACTGCCCGCTTTCCAG | |
| | TCGGGAAACCTGTCGTGCCAGCTGCATTAATGAATC | |
| | GGCCAACGCGCGGGGAGAGGCGGTTTGCGTATTGG | |
| | GCGCTCTTCCGCTTCCTCGCTCACTGACTCGCTGCGC | |
| | TCGGTCGTTCGGCTGCGGCGAGCGGTATCAGCTCAC | |
| | TCAAAGGCGGTAATACGGTTATCCACAGAATCAGG | |
| | GGATAACGCAGGAAAGAACATGTGAGCAAAAGGCC | |
| | AGCAAAAGCCCAGGAACCGTAAAAAGGCCGCGTTG | |
| | CTGGCGTTTTTCCATAGGCTCCGCCCCCCTGACGAG | |
| | CATCACAAAAATCGACGCTCAAGTCAGAGGTGGCG | |
| | AAACCCGACAGGACTATAAAGATACCAGGCGTTTC | |
| | CCCCTGGAAGCTCCCTCGTGCGCTCTCCTGTTCCGA | |
| | CCCTGCCGCTTACCGGATACCTGTCCGCCTTTCTCCC | |
| | TTCGGGAAGCGTGGCGCTTTCTCATAGCTCACGCTG | |
| | TAGGTATCTCAGTTCGGTGTAGGTCGTTCGCTCCAA | |
| | GCTGGGCTGTGTGCACGAACCCCCCGTTCAGCCCGA | |
| | CCGCTGCGCCTTATCCGGTAACTATCGTCTTGAGTC | |
| | CAACCCGGTAAGACACGACTTATCGCCACTGGCAG | |
| | CAGCCACTGGTAACAGGATTAGCAGAGCGAGGTAT | |
| | GTAGGCGGTGCTACAGAGTTCTTGAAGTGGTGGCCT | |
| | AACTACGGCTACACTAGAAGGACAGTATTTGGTATC | |
| | TGCGCTCTGCTGAAGCCAGTTACCTTCGGAAAAAGA | |
| | GTTGGTAGCTCTTGATCCGGCAAACAAACCACCGCT | |
| | GGTAGCGGTGGTTTTTTTGTTTGCAAGCAGCAGATT | |
| | ACGCGCAGAAAAAAAGGATCTCAAGAAGATCCTTT | |
| | GATCTTTTCTACGGGGTCTGACGCTCAGTGGAACGA | |
| | AAACTCACGTTAAGGGATTTTGGTCATGAGATTATC | |
| | AAAAAGGATCTTCACCTAGATCCTTTTAAATTAAAA | |
| | ATGAAGTTTTAAATCAATCTAAAGTATATATGAGTA | |
| | AACTTGGTCTGACAGTTACCAATGCTTAATCAGTGA | |
| | GGCACCTATCTCAGCGATCTGTCTATTTCGTTCATCC | |
| | ATAGTTGCCTGACTCCCCGTCGTGTAGATAACTACG | |
| | ATACGGGAGGGCTTACCATCTGGCCCCAGTGCTGCA | |
| | ATGATACCGCGAGACCCACGCTCACCGGCTCCAGAT | |
| | TTATCAGCAATAAACCAGCCAGCCGGAAGGGCCGA | |
| | GCGCAGAAGTGGTCCTGCAACTTTATCCGCCTCCAT | |
| | CCAGTCTATTAATTGTTGCCGGGAAGCTAGAGTAAG | |
| | TAGTTCGCCAGTTAATAGTTTGCGCAACGTTGTTGC | |
| | CATTGCTACAGGCATCGTGGTGTCACGCTCGTCGTT | |
| | TGGTATGGCTTCATTCAGCTCCGGTTCCCAACGATC | |
| | AAGGCGAGTTACATGATCCCCCATGTTGTGCAAAAA | |
| | AGCGGTTAGCTCCTTCGGTCCTCCGATCGTTGTCAG | |
| | AAGTAAGTTGGCCGCAGTGTTATCACTCATGGTTAT | |
| | GGCAGCACTGCATAATTCTCTTACTGTCATGCCATC | |
| | CGTAAGATGCTTTTCTGTGACTGGTGAGTACTCAAC | |
| | CAAGTCATTCTGAGAATAGTGTATGCGGCGACCGA | |
| | GTTGCTCTTGCCCGGCGTCAATACGGGATAATACCG | |
| | CGCCACATAGCAGAACTTTAAAAGTGCTCATCATTG | |
| | GAAAACGTTCTTCGGGGCGAAAACTCTCAAGGATCT | |
| | TACCGCTGTTGAGATCCAGTTCGATGTAACCCACTC | |
| | GTGCACCCAACTGATCTTCAGCATCTTTTACTTTCAC | |
| | CAGCGTTTCTGGGTGAGCAAAAACAGGAAGGCAAA | |
| | ATGCCGCAAAAAAGGGAATAAGGGCGACACGGAA | |
| | ATGTTGAATACTCATACTCTTCCTTTTTCAATATTAT | |
| | TGAAGCATTTATCAGGGTTATTGTCTCATGAGCGGA | |
| | TACATATTTGAATGTATTTAGAAAAATAAACAAATA | |
| | GGGGTTCCGCGCACATTTCCCCGAAAAGTGCCACCT | |
| | GTATGCGGTGTGAAATACCGCACAGATGCGTAAGG | |
| | AGAAAATACCGCATCAGGAAATTGTAAGCGTTAAT | |
| | AATTCAGAAGAACTCGTCAAGAAGGCGATAGAAGG | |
| | CGATGCGCTGCGAATCGGGAGCGGCGATACCGTAA | |
| | AGCACGAGGAAGCGGTCAGCCCATTCGCCGCCAAG | |
| | CTCTTCAGCAATATCACGGGTAGCCAACGCTATGTC | |
| | CTGATAG | |
| 2 | KEKNCPDGYIYSSNTASGYDCGVWICRRVGSAFCSRT | SARS2 spike |
| | GDYTSPSEFDIY | trimer binding |
| | | R4C1-DO1 |
| 3 | TCTGGCGCCATGGCCGGTTCTAAAGAAAAAAATTGT | SARS2 spike |
| | CCTGATGGCTATATCTATAGTTCTAATACCGCcAGC | trimer binding |
| | GGTTATGATTGTGGTGTCTGGATTTGTCGTCGCGTC | R4C1-DO1 |
| | GGTAGTGCCTTCTGTAGTCGTACTGGTGATTATACT | |
| | AGTCCTAGTGAATTTGACATTTACggttctCAGGAAAC | |
| | CTTTAGCGATCTGTGGAAACTGCTGCCGGAAAATCT | |
| | CGAGGGTGGTGGTGGGTCTCTAGCGGCCGCAACTG | |
| | ATCTTCACCA | |
| 4 | EGDKTCPDGYEHTCGCIGGCGCKRSACIGALCCQASL | SARS2 spike |
| | GGWLSDGETYT | RBD binding |
| | | R2G3-DO1 |
| 5 | TCTGGCGCCATGGCCGAAGGAGACAAAACGTGTCC | SARS2 spike |
| | TGATGGTTACGAGCATACTTGTGGTTGCATTGGGGG | RBD binding |
| | TTGTGGTTGCAAAAGGTCTGCCTGTATAGGTGCACT | R2G3-DO1 |
| | TTGTTGCCAAGCGTCGTTGGGTGGTTGGCTTAGTGA | |
| | CGGTGAAACCTACACTggttctCAGGAAACCTTTAGCG | |
| | ATCTGTGGAAACTGCTGCCGGAAAATGGTGGGTCTC | |
| | TAGCGGCCGCAACTGATCTTCACCA | |
| 6 | TCTGGCGCCATGGCCGGTTCTCAGGTTCAGCTGGTA | antibody scfv |
| | CAGTCTGGAGCTGAGGTGAAGAAGCCTGGGGCCTC | fragment |
| | AGTGAAGGTCTCCTGCAAGGCTTCTGGTTACACCTT | specific for |
| | TTCCAACTATGGTATCACCTGGGTGCGACAGGCCCC | αvβ3 integrin |
| | TGGACAAGGGCTTGAGTGGATGGGATGGATCAACA | |
| | ATGGTAACACACACTATGCACAGAAGTTCCAGGGC | |
| | AGAGTCACCATGACCACAGACACATCCACGAGCAC | |
| | AGCCTACATGGAGCTGAGGAGCCTTAGATCTGACG | |
| | ACACGGCCGTTTATTACTGTGCGAGAGACCCCCGGG | |
| | GTGACGACGAGCCCTACTGGGGCCAGGGAACCCTG | |
| | GTCACCGTCTCCTCAGGTTCTGGGGGTGGATCAGGT | |
| | GGAGGGGGTTCTGGTGGAGGTGGGTCTGAAATTGT | |
| | GTTGACGCAGTCTCCACTCTCCCTGCCCGTCACCCTT | |
| | GGACAGCCGGCCTCCATCTCCTGCCGGTCTAGTCAA | |
| | AACCTCGTATACAGTGATGGAAACACCTACTTGAGT | |
| | TGGTTTCAGCAGAGGCCAGGCCAATCTCCAAGGCG | |
| | CCTAATTTATAAGGTTTCTAACCGGGACTCTGGGGT | |
| | CCCAGACAGATTCAGTGGCAGTGGGTCAGGCACTG | |
| | ATTTCACACTGAAAATCAGCAGGGTGGAGGCTGAG | |
| | GATATTGGGGTCTATTACTGCATGCAAGGCACACAC | |
| | TGGCCTCCGCGGACGTTCGGCCAAGGGACCAAGCTT | |
| | GAGATCAAAggttctCAGGAAACCTTTAGCGATCTGTG | |
| | GAAACTGCTGCCGGAAAATCTCGAGGGTGGTGGTG | |
| | GGTCTCTAGCGGCCGCA | |
| 7 | GGTTCTGGCGCCATGGCCGGCTCCGAAGGTGACAA | Example 3 |
| | AACGTGTCCTGATNNBNNBNNBNNBNNBTGTNNBT | Library |
| | GCATTGGGGGTTGTGGTTGCNNBNNBNNBNNBTGTN | |
| | NBGGTNNBCTTTGTTGCNNBNNBNNBTTGGGTGGTT | |
| | GGCTTAGTGACCTCGAGGGTGGTGGTGGGTCTCTAG | |
| | CGGCCGCAACTGATTTCACT | |
| 8 | MVKNPNPVFTPREGAGTPKFREKPMEKAVGLTSRFDF | RepA sequence |
| | AIHVAHARSRGLRRRMPPVLRRRAIDALLQGLCFHYD | |
| | PLANRVQCSITTLAIECGLATESGAGKLSITRATRALTF | |
| | LSELGLITYQTEYDPLIGCYIPTDITFTLALFAALDV | |
| | SEDAVAAARRSRVEWENKQRKKQGLDTLGMDELIAK | |
| | AWRFVRERFRSYQTELQSRGIKRARARRDANRERQDI | |
| | VTLVKRQLTREISEGRFTANGEAVKREVERRVKERMI | |
| | LSRNRNYSRLATASP | |
| 9 | AAGTGATCTCCTCAGAATAATCCGGCCTGCGCCGGA | CIS |
| | GGCATCCGCACGCCTGAAGCCCGCCGGTGCACAAA | |
| | AAAACAGCGTCGCATGCAAAAAACAATCTCATCAT | |
| | CCACCTTCTGGAGCATCCGATTCCCCCTGTTTTTAAT | |
| | ACAAAATACGCCTCAGCGACGGGGAATTT | |
| 10 | TGCTTATCCACATTTAACTGCAAGGGACTTCCCCAT | ORI |
| | AAGGTTACAACCGTTCATGTCATAAAGCGCCAGCCG | |
| | CCAGTCTTACAGGGTGCAATGTATCTTTTAAACACC | |
| | TGTTTATATCTCCTTTAAACTACTTAATTACATTCAT | |
| | TTAAAAAGAAAACCTATTCACTGCCTGTCCTGTGGA | |
| | CAGACAGATATGCA | |
| 11 | atgcagggcg aagtcggaaa acttctgttc tgttaaatgt gttttgctca | trxA DNA |
| | tagtgtggta gaatatcagc ttactattgc tttacgaaag cgtatccggt | sequence |
| | gaaataaagt caacctttag ttggttaatg ttacaccaac aacgaaacca | |
| | acacgccagg cttattcctg tggagttata tatgagcgat aaaattattc | |
| | acctgactga cgacagtttt gacacggatg tactcaaagc ggacggggcg | |
| | atcctcgtcg atttctgggc agagtggtgc ggtccgtgca aaatgatcgc | |
| | cccgattctg gatgaaatcg ctgacgaata tcagggcaaa ctgaccgttg | |
| | caaaactgaa catcgatcaa aaccctggca ctgcgccgaa atatggcatc | |
| | cgtggtatcc cgactctgct gctgttcaaa aacggtgaag tggcggcaac | |
| | caaagtgggt gcactgtcta aaggtcagtt gaaagagttc ctcgacgcta | |
| | acctggcgta agggaatttc atgttcgggt gccccgtcgc taaaaactgg | |
| | acgccc | |
| 12 | GCGCAGTATGAAGATGGTAAACAGTACACTACCCT | DsbA DNA |
| | GGAAAAACCGGTAGCTGGCGCGCCGCAAGTGCTGG | sequence |
| | AGTTTTTCTCTTTCTTCTGCCCGCACTGCTATCAGTT | |
| | TGAAGAAGTTCTGCATATTTCTGATAATGTGAAGAA | |
| | AAAACTGCCGGAAGGCGTGAAGATGACTAAATACC | |
| | ACGTCAACTTCATGGGTGGTGACCTGGGCAAAGAcC | |
| | TGACTCAGGCATGGGCTGTGGCGATGGCGCTGGGC | |
| | GTGGAAGACAAAGTGACTGTTCCGCTGTTTGAAGGC | |
| | GTACAGAAAACCCAGACCATTCGTTCTGCTTCTGAT | |
| | ATCCGCGATGTATTTATCAACGCAGGTATTAAAGGT | |
| | GAAGAGTACGACGCGGCGTGGAACAGCTTCGTGGT | |
| | GAAATCTCTGGTCGCTCAGCAGGAAAAAGCTGCAG | |
| | CTGACGTGCAATTGCGTGGCGTTCCGGCGATGTTTG | |
| | TTAACGGTAAATATCAGCTGAATCCGCAGGGTATGG | |
| | ATACCAGCAATATGGATGTTTTTGTTCAGCAGTATG | |
| | CTGATACAGTGAAATACTTAAGCGAGAAAAAA | |
| 13 | MLYESVSGEIKSTFSWLMLHQQRNQHARLIPVELYMS | trxA protein |
| | DKIIHLTDDSFDTDVLKADGAILVDFWAEWCGPCKMI | sequence |
| | APILDEIADEYQGKLTVAKLNIDQNPGTAPKYGIRGIPT | |
| | LLLFKNGEVAATKVGALSKGQLKEFLDANLA | |
| 14 | MKKIWLALAGLVLAFSASAAQYEDGKQYTTLEKPVA | DsbA protein |
| | GAPQVLEFFSFFCPHCYQFEEVLHISDNVKKKLPEGVK | sequence |
| | MTKYHVNFMGGDLGKDLTQAWAVAMALGVEDKVT | |
| | VPLFEGVQKTQTIRSASDIRDVFINAGIKGEEYDAAWN | |
| | SFVVKSLVAQQEKAAADVQLRGVPAMFVNGKYQLNP | |
| | QGMDTSNMDVFVQQYADTVKYLSEKK | |
| 15 | GGGGS | Linker |
| 16 | CCGCTCTTCAGGGCACCCGAGTTCC | igGCDNAREV |
| 17 | GACACGCTGTCGCCATTCTGGTTCC | igACDNAREV |
| 18 | aaaaaaCTCGAGTCACCAGGCATCGACGTAGAATTC | p2pET32cloning |
| 19 | aaaaaaCTCGAGTCACCAGGCATCGACGAGCCATTG | p6PET32cloning |
| 20 | aaaaaaCTCGAGTCACCAGGCATCGACGTGCCATTC | p7pET32cloning |
| 21 | gtaatactcgagTCAGTGAAGATCAGTTGGGGCCgCTAG | CISXHOISTO |
| | | PRECREV |
| 22 | GGTTCTGGCGCCATGGCCGGCTCCGAAGG | G3LIBPTFOR |
| 23 | ATCAGTTGCGGCCGCTAGAGACCCACCACCACCCTC | G3LIBPTREV |
| | GAGGTCACTAAGCCAACCACCC | |
| 24 | GCTCTTCAGCAATATCACGGGTAGCCAACGCTATGTCC | FOR1 |
| 25 | CGGTCCGGACGTTGTAAAACGACGGCCAGTGAATTG | FOR2 |
| 26 | CaGCCGCGcATTCGCCCTTAACCGACTCTGACGGC | FOR3 |
| 27 | ACGAGAGAGATGATAGGGTCTGCTTCAGTAAGCCAG | FOR4 |
| 28 | ATGCTACACAATTAGGCTTGTAC | TACFAR6.2 |
| 29 | TGGTGAAGATCAGTTGCGGCCGCTAG | NOTRECREV4 |
| 30 | TGCATATCTGTCTGTCCACAGG | ORIREV |
| 31 | GCATCAAGGGCCCCAACTGATCTTCACCAAACGTAT | BSPREPAFOR |
| | TACC | |
| 32 | CAGCCGGCCATGGCCACATACTACAGTACTACTGTA | BovStalkFor2 |
| | TACC | |
| 33 | CAGCCGGCCATGGCCACATACTACAGTACTACTGTG | BovStalkFor3 |
| | CTCC | |
| 34 | CAGCCGGCCATGGCCACATACTACAGTGGTACTGTG | BovStalkFor4 |
| | CACC | |
| 35 | ttttttGCGGCCGCTAGAGAACCACCCCAGGCATCGACG | CISCOWREV2 |
| | TAGAATTC | |
| 36 | ttttttgcggccgctagagaaccaccCCAGGCATCGACGAGCCATTG | CISCOWREV6 |
| 37 | ttttttgcggccgctagagaaccaccCCAGGCATCGACGTGCCATTC | CISCOWREV7 |
| 38 | CTTVHQ | Base of Stalk A |
| 39 | CATVHQ | Base of Stalk A |
| 40 | CAIVQQ | Base of Stalk A |
| 41 | CATVDQ | Base of Stalk A |
| 42 | YX1YX2Y | Stalk B |
| | | X1 and X2 are |
| | | any amino acid |
| 43 | CX2TVX5Q | Ascending |
| | | Stalk Domain |
| | | X₂ and X₅ are |
| | | any amino acid |
| 44 | CX2TVX5Q | Ascending |
| | | Stalk Domain |
| | | X₂ is Ser, Thr, |
| | | Gly, Asn, Ala, |
| | | or Pro, and X₅ |
| | | is His, Gln, |
| | | Arg, Lys, Gly, |
| | | Thr, Tyr, Phe, |
| | | Trp, Met, Ile, |
| | | Val, or Leu |
| 45 | CX2TVX5Q | Ascending |
| | | Stalk Domain |
| | | X₂ is Ser, Ala, |
| | | or Thr, and X₅ |
| | | is His or Tyr |
| 46 | CCGCTCTTCAGGGCACCCGAGTTCC | igGCDNA2REV |
| 47 | CTGACTGTGCTGTTGTTGAACTTCC | igMCDNA2REV |
| 48 | GACACGCTGTCGCCATTCTGGTTCC | igACDNA2REV |
| 49 | CGGGCACGGTCACCATGCTGCTGAGAGAGTAG | igGCDNA1.7REV |
| 50 | TTACCTGCGGCCGCTGAGGAGACGGTGACCAGGAG | BOVVHFR4REV |
| | TCCAACTGGAGCTCCATCAAG | |
| 51 | CAGCCGGCCATGGCCACATACTACAGTACTACTGTA | BOVSTALKFOR1 |
| | CACC | |
| 52 | CAGCCGGCCATGGCCACATACTACAGTACTACTGTA | BOVSTALKFOR2 |
| | TACC | |
| 53 | CAGCCGGCCATGGCCACATACTACAGTACTACTGTG | BOVSTALKFOR3 |
| | CTCC | |
| 54 | CAGCCGGCCATGGCCACATACTACAGTGGTACTGTG | BOVSTALKFOR4 |
| | CACC | |
| 55 | ttacctgcggccgctgaggagacggtgaccaggagtcc | BOVVHFR4REV |
| 56 | ttttttgcggccgcccaggcgctgacgtaccattc | ULp1 |
| 57 | ttttttgcggccgcccaggcatcgacgtagaattc | ULp2 |
| 58 | ttttttgcggccgcccagacatcgacgaaaaattc | ULp3 |
| 59 | ttttttgcggccgcccaggcatggacgtaaaattg | ULp4 |
| 60 | ttttttgcggccgcccaagtctcgacataaaattc | ULp5 |
| 61 | ttttttgcggccgcccaggcatcgacgagccattg | ULp6 |
| 62 | ttttttgcggccgcccaggcatcgacgtgccattc | ULp7 |
| 63 | ttttttgcggccgcccaggcatcgacgtggaattc | ULp8 |
| 64 | ttttttgcggccgcccaggcatcgacgtggaagct | ULp9 |
| 65 | GGVCPKILQRCRRDSDSPGACICRGNGYCGSGSD | Mcoti-I |
| 66 | GGVCPKILKKCRRDSDSPGACICRGNGYCGSGSD | Mcoti-II |
| 67 | ERACPRILKKCRRDSDSPGACICRGNGYCG | Mcoti-III |
| 68 | TGCAGGTGCAGCTGCGGGAGTCGGG | Minimal |
| | | BOVVHNCOFOR2 |
| | | primer |
| 69 | TGAGGAGACGGTGACCAGGAGTCC | Minimal |
| | | BOVVHFR4X |
| | | HOREV |
| | | primer |
| 70 | AAAAAGCCATGGTGCAGGTGCAGCTGCGGGAGTCGGG | BOVVHNCOFOR2 |
| | | NotI restriction |
| | | enzyme site |
| | | (bold/underline) |
| 71 | TTACCTCTCGAGTGAGGAGACGGTGACCAGGAGTCC | BOVVHFR4XHOREV |
| | | Xho I |
| | | restriction |
| | | enzyme site |
| | | (bold/underline) |
| 72 | QAVLNQPSSVSGSLGQRVSITCSGSSSNVGNGYVSWYQLI | BLV5B8 |
| | PGSAPRTLIYGDTSRASGVPDRESGSRSGNTATLTISSL | Variable Light |
| | QAEDEADYFCASAEDSSSNAVFGSGTTLTVLGQP | Region |
| 73 | QAVLNQPSSVSGSLGQRVSITCSGSSSNVGNGYVSWYQLI | BLV1H12 |
| | PGSAPRTLIYGDTSRASGVPDRESGSRSGNTATLTISSL | variable Light |
| | QAEDEADYFCASAEDSSSNAVFGSGTTLTVL | chain |
| 74 | QAVLNQPSSVSGSLGQKVTISCSGSSSNIGNNYVSWYQQL | Humanized |
| | PGTAPKLLIYGDTKRPSGIPDRFSGSKSGTSATLGITGL | BLV1H12 |
| | QTGDEADYYCASAEDSSSNAVFGSGTTLTVLGQP | Variable Light |
| 75 | GlyGlyGlyGlySerGlyGlyGlyGlySerGlyGlyGlyGlySer | Flexible Linker |

What is claimed is:

1. A method of producing a disulfide rich protein (DRPs) CIS display library, the method comprising expressing a plurality of DNA constructs in a prokaryotic cell-free transcription/translation environment in the presence of at least one molecular chaperone,
wherein the at least one molecular chaperone is provided by (i) expression from a DNA sequence encoding the molecular chaperone included in the plurality of DNA constructs, or (ii) addition of one or more DNA sequences encoding the molecular chaperone or the molecular chaperone itself to the transcription/translation environment,
wherein each DNA construct comprises:
i. a DNA sequence encoding one of a plurality of disulfide rich protein (DRP), wherein each of the plurality of DRPs comprises a peptide of 20-70 amino acids in length having 2-12 cysteine residues capable of forming 1-6 intramolecular disulfide bonds;
ii. a DNA sequence encoding a DNA replication initiator protein; and
iii. a DNA sequence comprising a target sequence for the DNA replication,
thereby producing a plurality of protein fusions comprising the DRP and the DNA replication initiator protein and, optionally the molecular chaperone, from the plurality of DNA constructs,
wherein the protein fusion binds, via the DNA replication initiator protein, to the target sequence in the DNA construct from which it was transcribed, thereby linking the protein fusion to the DNA construct to produce a DRP CIS display library comprising a plurality of DNA:protein fusions.

2. The method of claim 1, wherein the molecular chaperone is provided by expression from a DNA sequence encoding the molecular chaperone included in each of the DNA constructs, and wherein each of the plurality of protein fusions comprises a DRP, the molecular chaperone, and the DNA replication initiator protein.

3. A method of producing a disulfide rich protein (DRPs) CIS display library, the method comprising:
(a) providing a plurality of DNA constructs each DNA construct comprising:
i. a DNA sequence encoding one of a plurality of disulfide rich proteins (DRPs), wherein each of the plurality of DRPs comprises a peptide of 20-70 amino acids in length having 2-12 cysteine residues capable of forming 1-6 intramolecular disulfide bonds;
ii. one or more DNA sequences encoding a molecular chaperone, wherein the one or more molecular chaperone is trxA;
iii. a DNA sequence encoding a DNA replication initiator protein, wherein the DNA replication initiator protein is RepA or a variant thereof that retains ability to bind to a target sequence that comprises an origin of replication sequence (ori); and
iv. a DNA sequence comprising the target sequence for the DNA replication initiator protein, wherein the DNA replication initiator protein can non-covalently bind to the target sequence; and
(b) expressing the plurality of DNA constructs of (a) in a prokaryotic cell-free transcription/translation environment, thereby producing a plurality of protein fusions comprising the DRP, the molecular chaperone and the DNA replication initiator protein from the plurality of DNA constructs, and
wherein the protein fusion binds, via the DNA replication initiator protein, to the target sequence in the DNA construct from which it was transcribed, thereby linking the protein fusion to the DNA construct to produce a DRP CIS display library comprising a plurality of DNA:protein fusion proteins.

4. The method of claim 1, wherein the method comprises the addition of one or more DNA sequences encoding the molecular chaperone or the molecular chaperone itself to the transcription/translation environment, wherein each of the plurality of protein fusions comprises a DRP and the DNA replication initiator protein.

5. The method of claim 1, wherein the plurality of DNA constructs comprise a DNA sequence encoding a different DRP from other members of the plurality of DNA constructs.

6. The method of claim 5, wherein the plurality of DRPs comprise DRP variants comprising one or more differences in their amino acid residues.

7. The method of claim 3, wherein the plurality of DNA constructs comprise a DNA sequence encoding a different DRP from other members of the plurality of DNA constructs.

8. The method of claim 7, wherein the plurality of DRPs comprise DRP variants comprising one or more differences in their amino acid residues.

9. The method of claim 1, wherein the peptide is a knob domain derived from an ultralong CDR3 of an antibody.

10. The method of claim 9, wherein the ultralong CDR3 of an antibody is from a member of the Bovinae subfamily.

11. The method of claim 3, wherein the peptide is knob domain derived from an ultralong CDR3 of an antibody.

12. The method of claim 11, wherein the ultralong CDR3 of an antibody is from a member of the Bovinae subfamily.

13. The method of claim 1, wherein the plurality of DRPs comprise at least 10³, at least 10⁵, at least 10⁷, or at least 10⁹ unique members, each member comprising a distinct DRP sequence.

14. The method of claim 3, wherein the plurality of DRPs comprise at least 10³, at least 10⁵, at least 10⁷, or at least 10⁹ unique members, each member comprising a distinct DRP sequence.

15. The method of claim 1, wherein the one or more encoded molecular chaperones comprise trxA, DsbA, DsbB, DsbC, HSP70, DnaK, HSP90, GroES, GroEL, DNAK, Sumo and Trigger Factor, or protein disulfide isomerase (PDI).

16. The method of claim 1, wherein the one or more molecular chaperone is the molecular chaperone trxA.

17. The method of claim 1, wherein the one or more molecular chaperone is the molecular chaperone DsbA.

18. The method of claim 3, wherein the DNA sequence encoding the one or more molecular chaperones is upstream of the DNA sequence encoding the DRP in the DNA construct.

19. The method of claim 3, wherein each of the plurality of DNA constructs comprises in order: the DNA sequence encoding the molecular chaperone, the DNA sequence encoding the DRP, the DNA sequence encoding the DNA replication initiator protein, and the DNA sequence encoding the target sequence for the DNA replication initiator protein.

20. The method of claim 1, wherein the DNA replication initiator protein is RepA or a variant thereof that retains ability to bind to the target sequence and the target sequence comprises an origin of replication sequence (ori).

21. The method of claim 20, wherein the wherein the DNA construct further comprises a cis-acting DNA element positioned between the DNA sequence encoding RepA and the target sequence comprising ori.

22. The method of claim 3, wherein the DNA construct further comprises a cis-acting DNA element positioned between the DNA sequence encoding RepA and the target sequence comprising ori.

23. The method of claim 3, wherein the RepA is selected from the group consisting of RepA of the incompatibility group I (IncI) complex plasmids and RepA of the IncF, IncB, IncK, IncZ and IncL/M plasmids.

24. A method of screening a disulfide rich protein (DRP) CIS display library, the method comprising:
(a) providing a screening library comprising a plurality of DNA:protein fusion proteins produced according to claim 1;
(b) contacting members of the plurality of DNA:protein fusion proteins of the screening library with a target of interest to produce a mixture of the plurality of DNA:protein fusion proteins and the target of interest; and
(c) selecting from the mixture the DNA:protein fusion proteins that are bound to the target of interest.

25. A method of screening a disulfide rich protein (DRP) CIS display library, the method comprising:
(a) providing a screening library comprising a plurality of DNA:protein fusion proteins produced according to claim 3;
(b) contacting members of the plurality of DNA:protein fusion proteins of the screening library with a target of interest to produce a mixture of the plurality of DNA:protein fusion proteins and the target of interest; and
(c) selecting from the mixture the DNA:protein fusion proteins that are bound to the target of interest.

26. The method of claim 24, further comprising identifying the one or more DNA:protein fusion proteins bound to the target of interest by identifying a sequence of the corresponding DNA:protein fusion replication initiator protein DNA.

27. The method of claim 26, wherein identifying the sequence of the corresponding DNA:protein fusion replication initiator protein DNA comprises nucleic acid sequencing.

28. The method of claim 25, further comprising identifying the one or more DNA:protein fusion proteins bound to the target of interest by identifying a sequence of the corresponding DNA:protein fusion replication initiator protein DNA.

29. The method of claim 28, wherein identifying the sequence of the corresponding DNA:protein fusion replication initiator protein DNA comprises nucleic acid sequencing.


SEQUENCE LISTING (75 sequences)

SEQ ID NO: 1 - DNA, 4416 bp, synthetic construct
  source 1..4416: mol_type = other DNA
  misc_feature 1..4416: map = DNA vector pREPTRX
cggtccggacgttgtaaaacgacggccagtgaattgtaatacgactcactatagggcgaattgaatttagcagccgcgcattcgcccttaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacaagcttactccccatccccctgttgacaattaatcatggctcgtataatgtgtggaattgtgagcggataacaatttcacacaggaaacaggatctaccatgagcgataaaattattcacctgactgacgacagttttgacacggatgtactcaaagcggacggggcgatcctcgtcgatttctgggcagagtggtgtggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggccggttctggttctggcgccatggcctaacgagctcatataactcgagggtggtggtgggtctctagcgggccccaactgatcttcaccaaacgtattaccgccaggtaaagaacccgaatccggtgttcactccccgtgaaggtgccggaacgctgaagttctgcgaaaaactgatggaaaaggcggtgggcttcacctcccgttttgatttcgccattcatgtggcgcatgcccgttcccgtggtctgcgtcggcgcatgccaccggtgctgcgtcgacgggctattgatgcgctgctgcaggggctgtgtttccactatgacccgctggccaaccgcgtccagtgttccatcaccacactggccattgagtgcggactggcgacagagtccggtgcaggaaaactctccatcacccgtgccacccgtgccctgacgttcctgtcagagctgggactgattacctaccagacggaatatgacccgcttatcgggtgctacattccgaccgacatcacgttcacactggctctgtttgctgcccttgatgtgtctgaggatgcagtggcagctgcgcgccgcagtcgtgttgaatgggaaaacaaacagcgcaaaaagcaggggctggataccctgggtatggatgagctgatagcgaaagcctggcgttttgtgcgtgagcgtttccgcagttaccagacagagcttaagtcccgtggaataaaacgtgcccgtgcgcgtcgtgatgcgaacagagaacgtcaggatatcgtcaccctggtgaaacggcagctgacgcgcgaaatctcggaaggacgcttcactgctaatggtgaggcggtaaaacgcgaagtggagcgtcgtgtgaaggagcgcatgattctgtcacgtaaccgcaattacagccggctggccacagcttctccctgaaagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattttgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgcaaagggcgaattcgtttaaacctgcaggactagtccctttagtgagggttaattctgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaagcccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgtatgcggtgtgaaataccgcacagatgcgtaaggagaaaataccgcatcaggaaattgtaagcgttaataattcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatag

SEQ ID NO: 2 - protein, 49 aa, synthetic construct
  REGION 1..49: note = SARS2 spike trimer binding R4C1-DO1
KEKNCPDGYIYSSNTASGYDCGVWICRRVGSAFCSRTGDYTSPSEFDIY

SEQ ID NO: 3 - DNA, 263 bp, synthetic construct
  source 1..263: mol_type = other DNA
  misc_feature 1..263: note = SARS2 spike trimer binding R4C1-DO1
tctggcgccatggccggttctaaagaaaaaaattgtcctgatggctatatctatagttctaataccgccagcggttatgattgtggtgtctggatttgtcgtcgcgtcggtagtgccttctgtagtcgtactggtgattatactagtcctagtgaatttgacatttacggttctcaggaaacctttagcgatctgtggaaactgctgccggaaaatctcgagggtggtggtgggtctctagcggccgcaactgatcttcacca

SEQ ID NO: 4 - protein, 48 aa, synthetic construct
  REGION 1..48: note = SARS2 spike RBD binding R2G3-DO1
EGDKTCPDGYEHTCGCIGGCGCKRSACIGALCCQASLGGWLSDGETYT

SEQ ID NO: 5 - DNA, 242 bp, synthetic construct
  source 1..242: mol_type = other DNA
  misc_feature 1..242: note = SARS2 spike RBD binding R2G3-DO1
tctggcgccatggccgaaggagacaaaacgtgtcctgatggttacgagcatacttgtggttgcattgggggttgtggttgcaaaaggtctgcctgtataggtgcactttgttgccaagcgtcgttgggtggttggcttagtgacggtgaaacctacactggttctcaggaaacctttagcgatctgtggaaactgctgccggaaaatggtgggtctctagcggccgcaactgatcttcacca

SEQ ID NO: 6 - DNA, 837 bp, synthetic construct
  source 1..837: mol_type = other DNA
  misc_feature 1..837: note = antibody scfv fragment specific for avb3 integrin
tctggcgccatggccggttctcaggttcagctggtacagtctggagctgaggtgaagaagcctggggcctcagtgaaggtctcctgcaaggcttctggttacaccttttccaactatggtatcacctgggtgcgacaggcccctggacaagggcttgagtggatgggatggatcaacaatggtaacacacactatgcacagaagttccagggcagagtcaccatgaccacagacacatccacgagcacagcctacatggagctgaggagccttagatctgacgacacggccgtttattactgtgcgagagacccccggggtgacgacgagccctactggggccagggaaccctggtcaccgtctcctcaggttctgggggtggatcaggtggagggggttctggtggaggtgggtctgaaattgtgttgacgcagtctccactctccctgcccgtcacccttggacagccggcctccatctcctgccggtctagtcaaaacctcgtatacagtgatggaaacacctacttgagttggtttcagcagaggccaggccaatctccaaggcgcctaatttataaggtttctaaccgggactctggggtcccagacagattcagtggcagtgggtcaggcactgatttcacactgaaaatcagcagggtggaggctgaggatattggggtctattactgcatgcaaggcacacactggcctccgcggacgttcggccaagggaccaagcttgagatcaaaggttctcaggaaacctttagcgatctgtggaaactgctgccggaaaatctcgagggtggtggtgggtctctagcggccgca

SEQ ID NO: 7 - DNA, 198 bp, synthetic construct
  source 1..198: mol_type = other DNA
  misc_structure 1..198: note = Example 3 Library
  variation 49..132: note = b = c, g, t/u, but not a
  variation 49..132: note = n = any nucleotide
ggttctggcgccatggccggctccgaaggtgacaaaacgtgtcctgatnnbnnbnnbnnbnnbtgtnnbtgcattgggggttgtggttgcnnbnnbnnbnnbtgtnnbggtnnbctttgttgcnnbnnbnnbttgggtggttggcttagtgacctcgagggtggtggtgggtctctagcggccgcaactgatttcact

SEQ ID NO: 8 - protein, 275 aa, synthetic construct
  REGION 1..275: note = RepA sequence
MVKNPNPVFTPREGAGTPKFREKPMEKAVGLTSRFDFAIHVAHARSRGLRRRMPPVLRRRAIDALLQGLCFHYDPLANRVQCSITTLAIECGLATESGAGKLSITRATRALTFLSELGLITYQTEYDPLIGCYIPTDITFTLALFAALDVSEDAVAAARRSRVEWENKQRKKQGLDTLGMDELIAKAWRFVRERFRSYQTELQSRGIKRARARRDANRERQDIVTLVKRQLTREISEGRFTANGEAVKREVERRVKERMILSRNRNYSRLATASP

SEQ ID NO: 9 - DNA, 172 bp, synthetic construct
  source 1..172: mol_type = other DNA
  misc_feature 1..172: note = CIS
aagtgatctcctcagaataatccggcctgcgccggaggcatccgcacgcctgaagcccgccggtgcacaaaaaaacagcgtcgcatgcaaaaaacaatctcatcatccaccttctggagcatccgattccccctgtttttaatacaaaatacgcctcagcgacggggaattt

SEQ ID NO: 10 - DNA, 195 bp, synthetic construct
  source 1..195: mol_type = other DNA
  misc_feature 1..195: note = ORI
tgcttatccacatttaactgcaagggacttccccataaggttacaaccgttcatgtcataaagcgccagccgccagtcttacagggtgcaatgtatcttttaaacacctgtttatatctcctttaaactacttaattacattcatttaaaaagaaaacctattcactgcctgtcctgtggacagacagatatgca

SEQ ID NO: 11 - DNA, 556 bp, synthetic construct
  source 1..556: mol_type = other DNA
  misc_feature 1..556: note = trxA DNA sequence
atgcagggcgaagtcggaaaacttctgttctgttaaatgtgttttgctcatagtgtggtagaatatcagcttactattgctttacgaaagcgtatccggtgaaataaagtcaacctttagttggttaatgttacaccaacaacgaaaccaacacgccaggcttattcctgtggagttatatatgagcgataaaattattcacctgactgacgacagttttgacacggatgtactcaaagcggacggggcgatcctcgtcgatttctgggcagagtggtgcggtccgtgcaaaatgatcgccccgattctggatgaaatcgctgacgaatatcagggcaaactgaccgttgcaaaactgaacatcgatcaaaaccctggcactgcgccgaaatatggcatccgtggtatcccgactctgctgctgttcaaaaacggtgaagtggcggcaaccaaagtgggtgcactgtctaaaggtcagttgaaagagttcctcgacgctaacctggcgtaagggaatttcatgttcgggtgccccgtcgctaaaaactggacgccc

SEQ ID NO: 12 - DNA, 567 bp, synthetic construct
  source 1..567: mol_type = other DNA
  misc_feature 1..567: note = DsbA DNA sequence
gcgcagtatgaagatggtaaacagtacactaccctggaaaaaccggtagctggcgcgccgcaagtgctggagtttttctctttcttctgcccgcactgctatcagtttgaagaagttctgcatatttctgataatgtgaagaaaaaactgccggaaggcgtgaagatgactaaataccacgtcaacttcatgggtggtgacctgggcaaagacctgactcaggcatgggctgtggcgatggcgctgggcgtggaagacaaagtgactgttccgctgtttgaaggcgtacagaaaacccagaccattcgttctgcttctgatatccgcgatgtatttatcaacgcaggtattaaaggtgaagagtacgacgcggcgtggaacagcttcgtggtgaaatctctggtcgctcagcaggaaaaagctgcagctgacgtgcaattgcgtggcgttccggcgatgtttgttaacggtaaatatcagctgaatccgcagggtatggataccagcaatatggatgtttttgttcagcagtatgctgatacagtgaaatacttaagcgagaaaaaa

SEQ ID NO: 13 - protein, 144 aa, synthetic construct
  REGION 1..144: note = trxA protein sequence
MLYESVSGEIKSTFSWLMLHQQRNQHARLIPVELYMSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 14 - protein, 208 aa, synthetic construct
  REGION 1..208: note = DsbA protein sequence
MKKIWLALAGLVLAFSASAAQYEDGKQYTTLEKPVAGAPQVLEFFSFFCPHCYQFEEVLHISDNVKKKLPEGVKMTKYHVNFMGGDLGKDLTQAWAVAMALGVEDKVTVPLFEGVQKTQTIRSASDIRDVFINAGIKGEEYDAAWNSFVVKSLVAQQEKAAADVQLRGVPAMFVNGKYQLNPQGMDTSNMDVFVQQYADTVKYLSEKK

SEQ ID NO: 15 - protein, 5 aa, synthetic construct
  REGION 1..5: note = Linker
GGGGS

SEQ ID NO: 16 - DNA, 25 bp, synthetic construct
  source 1..25: mol_type = other DNA
  misc_feature 1..25: note = igGCDNAREV
ccgctcttcagggcacccgagttcc

SEQ ID NO: 17 - DNA, 25 bp, synthetic construct
  source 1..25: mol_type = other DNA
  misc_feature 1..25: note = igACDNAREV
gacacgctgtcgccattctggttcc

SEQ ID NO: 18 - DNA, 36 bp, synthetic construct
  source 1..36: mol_type = other DNA
  misc_feature 1..36: note = p2pET32cloning
aaaaaactcgagtcaccaggcatcgacgtagaattc

SEQ ID NO: 19 - DNA, 36 bp, synthetic construct
  source 1..36: mol_type = other DNA
  misc_feature 1..36: note = p6PET32cloning
aaaaaactcgagtcaccaggcatcgacgagccattg

SEQ ID NO: 20 - DNA, 36 bp, synthetic construct
  source 1..36: mol_type = other DNA
  misc_feature 1..36: note = p7pET32cloning
aaaaaactcgagtcaccaggcatcgacgtgccattc

SEQ ID NO: 21 - DNA, 39 bp, synthetic construct
  source 1..39: mol_type = other DNA
  misc_feature 1..39: note = CISXHOISTOPRECREV
gtaatactcgagtcagtgaagatcagttggggccgctag

SEQ ID NO: 22 - DNA, 29 bp, synthetic construct
  source 1..29: mol_type = other DNA
  misc_feature 1..29: note = G3LIBPTFOR
ggttctggcgccatggccggctccgaagg

SEQ ID NO: 23 - DNA, 58 bp, synthetic construct
  source 1..58: mol_type = other DNA
  misc_feature 1..58: note = G3LIBPTREV
atcagttgcggccgctagagacccaccaccaccctcgaggtcactaagccaaccaccc

SEQ ID NO: 24 - DNA, 38 bp, synthetic construct
  source 1..38: mol_type = other DNA
  misc_feature 1..38: note = FOR1
gctcttcagcaatatcacgggtagccaacgctatgtcc

SEQ ID NO: 25 - DNA, 36 bp, synthetic construct
  source 1..36: mol_type = other DNA
  misc_feature 1..36: note = FOR2
cggtccggacgttgtaaaacgacggccagtgaattg

SEQ ID NO: 26 - DNA, 35 bp, synthetic construct
  source 1..35: mol_type = other DNA
  misc_feature 1..35: note = FOR3
cagccgcgcattcgcccttaaccgactctgacggc

SEQ ID NO: 27 - DNA, 36 bp, synthetic construct
  source 1..36: mol_type = other DNA
  misc_feature 1..36: note = FOR4
acgagagagatgatagggtctgcttcagtaagccag

SEQ ID NO: 28 - DNA, 23 bp, synthetic construct
  source 1..23: mol_type = other DNA
  misc_feature 1..23: note = TACFAR6.2
atgctacacaattaggcttgtac

SEQ ID NO: 29 - DNA, 26 bp, synthetic construct
  source 1..26: mol_type = other DNA
  misc_feature 1..26: note = NOTRECREV4
tggtgaagatcagttgcggccgctag

SEQ ID NO: 30 - DNA, 22 bp, synthetic construct
  source 1..22: mol_type = other DNA
  misc_feature 1..22: note = ORIREV
tgcatatctgtctgtccacagg

SEQ ID NO: 31 - DNA, 40 bp, synthetic construct
  source 1..40: mol_type = other DNA
  misc_feature 1..40: note = BSPREPAFOR
gcatcaagggccccaactgatcttcaccaaacgtattacc

SEQ ID NO: 32 - DNA, 40 bp, synthetic construct
  source 1..40: mol_type = other DNA
  misc_feature 1..40: note = BovStalkFor2
cagccggccatggccacatactacagtactactgtatacc

SEQ ID NO: 33 - DNA, 40 bp, synthetic construct
  source 1..40: mol_type = other DNA
  misc_feature 1..40: note = BovStalkFor3
cagccggccatggccacatactacagtactactgtgctcc

SEQ ID NO: 34 - DNA, 40 bp, synthetic construct
  source 1..40: mol_type = other DNA
  misc_feature 1..40: map = BovStalkFor4
cagccggccatggccacatactacagtggtactgtgcacc

SEQ ID NO: 35 - DNA, 47 bp, synthetic construct
  source 1..47: mol_type = other DNA
  misc_feature 1..47: note = CISCOWREV2
ttttttgcggccgctagagaaccaccccaggcatcgacgtagaattc

SEQ ID NO: 36 - DNA, 47 bp, synthetic construct
  source 1..47: mol_type = other DNA
  misc_feature 1..47: note = CISCOWREV6
ttttttgcggccgctagagaaccaccccaggcatcgacgagccattg

SEQ ID NO: 37 - DNA, 47 bp, synthetic construct
  source 1..47: mol_type = other DNA
  misc_feature 1..47: note = CISCOWREV7
ttttttgcggccgctagagaaccaccccaggcatcgacgtgccattc

SEQ ID NO: 38 - protein, 6 aa, synthetic construct
  REGION 1..6: note = Base of Stalk A
CTTVHQ

SEQ ID NO: 39 - protein, 6 aa, synthetic construct
  REGION 1..6: note = Base of Stalk A
CATVHQ

SEQ ID NO: 40 - protein, 6 aa, synthetic construct
  REGION 1..6: note = Base of Stalk A
CAIVQQ

SEQ ID NO: 41 - protein, 6 aa, synthetic construct
  REGION 1..6: note = Base of Stalk A
CATVDQ

SEQ ID NO: 42 - skipped

SEQ ID NO: 43 - protein, 6 aa, synthetic construct
  REGION 1..6: note = Ascending Stalk Domain
  VARIANT 2: note = X is any amino acid
  VARIANT 4: note = X is any amino acid
CXTVXQ

SEQ ID NO: 44 - protein, 6 aa, synthetic construct
  REGION 1..6: note = Ascending Stalk Domain
  VARIANT 2: note = X is Ser, Thr, Gly, Asn, Ala, or Pro
  VARIANT 5: note = X is His, Gln, Arg, Lys, Gly, Thr, Tyr, Phe, Trp, Met, Ile, Val, or Leu
CXTVXQ

SEQ ID NO: 45 - protein, 6 aa, synthetic construct
  REGION 1..6: note = Ascending Stalk Domain
  VARIANT 2: note = X is Ser, Ala, or Thr, and X5 is His or Tyr
  VARIANT 5: note = X is Ser, Ala, or Thr, and X5 is His or Tyr
CXTVXQ

SEQ ID NO: 46 - DNA, 25 bp, synthetic construct
  source 1..25: mol_type = other DNA
  misc_feature 1..25: note = igGCDNA2REV
ccgctcttcagggcacccgagttcc

SEQ ID NO: 47 - DNA, 25 bp, synthetic construct
  source 1..25: mol_type = other DNA
  misc_feature 1..25: note = igMCDNA2REV
ctgactgtgctgttgttgaacttcc

SEQ ID NO: 48 - DNA, 25 bp, synthetic construct
  source 1..25: mol_type = other DNA
  misc_feature 1..25: note = igACDNA2REV
gacacgctgtcgccattctggttcc

SEQ ID NO: 49 - DNA, 32 bp, synthetic construct
  source 1..32: mol_type = other DNA
  misc_feature 1..32: note = igGCDNA1.7REV
cgggcacggtcaccatgctgctgagagagtag

SEQ ID NO: 50 - DNA, 56 bp, synthetic construct
  source 1..56: mol_type = other DNA
  misc_feature 1..56: note = BOVVHFR4REV
ttacctgcggccgctgaggagacggtgaccaggagtccaactggagctccatcaag

SEQ ID NO: 51 - DNA, 40 bp, synthetic construct
  source 1..40: mol_type = other DNA
  misc_feature 1..40: note = BOVSTALKFOR1
cagccggccatggccacatactacagtactactgtacacc

SEQ ID NO: 52 - DNA, 40 bp, synthetic construct; identical to SEQ ID NO: 32
  source 1..40: mol_type = other DNA
  misc_feature 1..40: note = BOVSTALKFOR2

SEQ ID NO: 53 - DNA, 40 bp, synthetic construct; identical to SEQ ID NO: 33
  source 1..40: mol_type = other DNA
  misc_feature 1..40: note = BOVSTALKFOR3

SEQ ID NO: 54 - DNA, 40 bp, synthetic construct; identical to SEQ ID NO: 34
  source 1..40: mol_type = other DNA
  misc_feature 1..40: note = BOVSTALKFOR4

SEQ ID NO: 55 - DNA, 38 bp, synthetic construct
  source 1..38: mol_type = other DNA
  misc_feature 1..38: note = BOVVHFR4REV
ttacctgcggccgctgaggagacggtgaccaggagtcc

SEQ ID NO: 56 - DNA, 35 bp, synthetic construct
  source 1..35: mol_type = other DNA
  misc_feature 1..35: note = ULp1
ttttttgcggccgcccaggcgctgacgtaccattc

SEQ ID NO: 57 - DNA, 35 bp, synthetic construct
  source 1..35: mol_type = other DNA
  misc_feature 1..35: note = ULp2
ttttttgcggccgcccaggcatcgacgtagaattc

SEQ ID NO: 58 - DNA, 35 bp, synthetic construct
  source 1..35: mol_type = other DNA
  misc_feature 1..35: note = ULp3
ttttttgcggccgcccagacatcgacgaaaaattc

SEQ ID NO: 59 - DNA, 35 bp, synthetic construct
  source 1..35: mol_type = other DNA
  misc_feature 1..35: note = ULp4
ttttttgcggccgcccaggcatggacgtaaaattg

SEQ ID NO: 60 - DNA, 35 bp, synthetic construct
  source 1..35: mol_type = other DNA
  misc_feature 1..35: note = ULp5
ttttttgcggccgcccaagtctcgacataaaattc

SEQ ID NO: 61 - DNA, 35 bp, synthetic construct
  source 1..35: mol_type = other DNA
  misc_feature 1..35: note = ULp6
ttttttgcggccgcccaggcatcgacgagccattg

SEQ ID NO: 62 - DNA, 35 bp, synthetic construct
  source 1..35: mol_type = other DNA
  misc_feature 1..35: note = ULp7
ttttttgcggccgcccaggcatcgacgtgccattc

SEQ ID NO: 63 - DNA, 35 bp, synthetic construct
  source 1..35: mol_type = other DNA
  misc_feature 1..35: note = ULp8
ttttttgcggccgcccaggcatcgacgtggaattc

SEQ ID NO: 64 - DNA, 35 bp, synthetic construct
  source 1..35: mol_type = other DNA
  misc_feature 1..35: note = ULp9
ttttttgcggccgcccaggcatcgacgtggaagct

SEQ ID NO: 65 - protein, 34 aa, synthetic construct
  REGION 1..34: note = Mcoti-I
GGVCPKILQRCRRDSDSPGACICRGNGYCGSGSD

SEQ ID NO: 66 - protein, 34 aa, synthetic construct
  REGION 1..34: note = Mcoti-II
GGVCPKILKKCRRDSDSPGACICRGNGYCGSGSD

SEQ ID NO: 67 - protein, 30 aa, synthetic construct
  REGION 1..30: note = Mcoti-III
ERACPRILKKCRRDSDSPGACICRGNGYCG

SEQ ID NO: 68 - DNA, 25 bp, synthetic construct
  source 1..25: mol_type = other DNA
  misc_feature 1..25: note = Minimal BOVVHNCOFOR2 primer
tgcaggtgcagctgcgggagtcggg

SEQ ID NO: 69 - DNA, 24 bp, synthetic construct
  source 1..24: mol_type = other DNA
  misc_feature 1..24: note = Minimal BOVVHFR4XHOREV primer
tgaggagacggtgaccaggagtcc

SEQ ID NO: 70 - DNA, 37 bp, synthetic construct
  source 1..37: mol_type = other DNA
  misc_feature 1..37: note = BOVVHNCOFOR2
  misc_structure 7..12: note = NotI restriction enzyme site
aaaaagccatggtgcaggtgcagctgcgggagtcggg

SEQ ID NO: 71 - DNA, 36 bp, synthetic construct
  source 1..36: mol_type = other DNA
  misc_feature 1..36: note = BOVVHFR4XHOREV
  misc_structure 7..12: note = Xho I restriction enzyme site
ttacctctcgagtgaggagacggtgaccaggagtcc

SEQ ID NO: 72 - protein, 113 aa, synthetic construct
  REGION 1..113: note = BLV5B8 Variable Light Region
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVLGQP

SEQ ID NO: 73 - protein, 110 aa, synthetic construct
  REGION 1..110: note = BLV1H12 variable Light chain
QAVLNQPSSVSGSLGQRVSITCSGSSSNVGNGYVSWYQLIPGSAPRTLIYGDTSRASGVPDRFSGSRSGNTATLTISSLQAEDEADYFCASAEDSSSNAVFGSGTTLTVL

SEQ ID NO: 74 - protein, 113 aa, synthetic construct
  REGION 1..113: note = Humanized BLV1H12 Variable Light
QAVLNQPSSVSGSLGQKVTISCSGSSSNIGNNYVSWYQQLPGTAPKLLIYGDTKRPSGIPDRFSGSKSGTSATLGITGLQTGDEADYYCASAEDSSSNAVFGSGTTLTVLGQP

SEQ ID NO: 75 - protein, 15 aa, synthetic construct
  REGION 1..15: note = Flexible Linker
GGGGSGGGGSGGGGS